SEQUENCE LISTING (106 sequences)

SEQ ID NO: 1 - protein, 5 aa, synthetic construct
SYTMS

SEQ ID NO: 2 - protein, 17 aa, synthetic construct
TISGGGRDIYYPDSVKG

SEQ ID NO: 3 - protein, 11 aa, synthetic construct
LTGRVYFALDS

SEQ ID NO: 4 - protein, 15 aa, synthetic construct
KASESVDTSDNSFIH

SEQ ID NO: 5 - protein, 7 aa, synthetic construct
RSSTLES

SEQ ID NO: 6 - protein, 9 aa, synthetic construct
QQNYDVPWT

SEQ ID NO: 7 - protein, 120 aa, synthetic construct
EVILVESGGGLVKPGGSLKLSCAASGFSFSSYTMSWVRQTPEKRLDWVATISGGGRDIYYPDSVKGRFTISRDNAKNTLYLEMSSLMSEDTALYYCVLLTGRVYFALDSWGQGTSVTVSS

SEQ ID NO: 8 - protein, 111 aa, synthetic construct
KIVLTQSPASLPVSLGQRATISCRASESVDTSDNSFIHWYQQRPGQSPKLLIYRSSTLESGVPARFSGSGSRTDFTLTIDPVEADDVATYYCQQNYDVPWTFGGGTKLEIK

SEQ ID NO: 9 - protein, 120 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSS

SEQ ID NO: 10 - protein, 111 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIK

SEQ ID NO: 11 - protein, 5 aa, synthetic construct
DYTMN

SEQ ID NO: 12 - protein, 17 aa, synthetic construct
VISWDGGGTYYTDSVKG

SEQ ID NO: 13 - protein, 12 aa, synthetic construct
GLTDTTLYGSDY

SEQ ID NO: 14 - protein, 11 aa, synthetic construct
RASQSISSYLN

SEQ ID NO: 15 - protein, 7 aa, synthetic construct
AASTLQS

SEQ ID NO: 16 - protein, 9 aa, synthetic construct
QQTYSSPLT

SEQ ID NO: 17 - protein, 121 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSS

SEQ ID NO: 18 - protein, 107 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIK

SEQ ID NO: 19 - protein, 5 aa, synthetic construct
DYAMS

SEQ ID NO: 20 - protein, 17 aa, synthetic construct
GIDNSGYYTYYTDSVKG

SEQ ID NO: 21 - protein, 13 aa, synthetic construct
THSGLIVNDAFDI

SEQ ID NO: 22 - protein, 11 aa, synthetic construct
RASQSISSYLN

SEQ ID NO: 23 - protein, 7 aa, synthetic construct
DASSLES

SEQ ID NO: 24 - protein, 9 aa, synthetic construct
QQSYSTPLT

SEQ ID NO: 25 - protein, 122 aa, synthetic construct
EVQLVESGGGLVQPGGSLRLACAASGFTFSDYAMSWVRQAPGKGLEWVSGIDNSGYYTYYTDSVKGRFTISRDDVKNTLYLQMNSLRAEDTAVYLCTKTHSGLIVNDAFDIWGQGTMVTVSS

SEQ ID NO: 26 - protein, 107 aa, synthetic construct
DIQLTQSPSSLSASVGDRVTITCRASQSISSYLNWYQQKPGKAPKLLIYDASSLESGVPSRFSGSGSGTDATLTISSLQPEDFATYYCQQSYSTPLTFGGGTKVEIK

SEQ ID NO: 27 - protein, 120 aa, synthetic construct
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 28 - protein, 107 aa, synthetic construct
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 29 - protein, 120 aa, synthetic construct
QVQLQQWGAGLLKPSETLSLTCAVYGGSFSDYYWNWIRQPPGKGLEWIGEINHNGNTNSNPSLKSRVTLSLDTSKNQFSLKLRSVTAADTAVYYCAFGYSDYEYNWFDPWGQGTLVTVSS

SEQ ID NO: 30 - protein, 107 aa, synthetic construct; identical to SEQ ID NO: 28

SEQ ID NO: 31 - protein, 125 aa, synthetic construct
QVQLVQSGAEVKKPGASVKVSCKASGFTLTNYGMNWVRQARGQRLEWIGWINTDTGEPTYADDFKGRFVFSLDTSVSTAYLQISSLKAEDTAVYYCARNPPYYYGTNNAEAMDYWGQGTTVTVSS

SEQ ID NO: 32 - protein, 107 aa, synthetic construct
DIQMTQSPSSLSASVGDRVTITCSSSQDISNYLNWYLQKPGQSPQLLIYYTSTLHLGVPSRFSGSGSGTEFTLTISSLQPDDFATYYCQQYYNLPWTFGQGTKVEIK

SEQ ID NO: 33 - protein, 122 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSS

SEQ ID NO: 34 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 35 - protein, 441 aa, synthetic construct
DIVMTQSPDSLAVSLGERATINCKASESVDTSDNSFIHWYQQKPGQSPKLLIYRSSTLESGVPDRFSGSGSGTDFTLTISSLQAEDVAVYYCQQNYDVPWTFGQGTKVEIKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 36 - protein, 227 aa, synthetic construct
EVQLLESGGGLVQPGGSLRLSCAASGFSFSSYTMSWVRQAPGKGLEWVATISGGGRDIYYPDSVKGRFTISRDNSKNTLYLQMNSLRAEDTAVYYCVLLTGRVYFALDSWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 37 - protein, 449 aa, synthetic construct
EVQLLESGGGLVQPGGSLRLSCAASGFIFDDYTMNWVRQAPGKGLEWVAVISWDGGGTYYTDSVKGRFTISRDDFKNTLYLQMNSLRAEDTAVYYCAKGLTDTTLYGSDYWGQGTLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 38 - protein, 214 aa, synthetic construct
DIQMTQSPSSLSASVGDRVTITCRASQSISSYLNWYQQKPGKAPKLLIYAASTLQSGVPSRFSGSGSGTDFTLTISSLQPEDFATYYCQQTYSSPLTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 39 - protein, 450 aa, synthetic construct
EVQLVESGGGLVQPGGSLRLACAASGFTFSDYAMSWVRQAPGKGLEWVSGIDNSGYYTYYTDSVKGRFTISRDDVKNTLYLQMNSLRAEDTAVYLCTKTHSGLIVNDAFDIWGQGTMVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 40 - protein, 214 aa, synthetic construct
DIQLTQSPSSLSASVGDRVTITCRASQSISSYLNWYQQKPGKAPKLLIYDASSLESGVPSRFSGSGSGTDATLTISSLQPEDFATYYCQQSYSTPLTFGGGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 5 aa, synthetic construct
SYAMN

SEQ ID NO: 42 - protein, 19 aa, synthetic construct
RIRSKYNNYATYYADSVKG

SEQ ID NO: 43 - protein, 14 aa, synthetic construct
ASNFPASYVSYFAY

SEQ ID NO: 44 - protein, 14 aa, unidentified
GSSTGAVTTSNYAN

SEQ ID NO: 45 - protein, 7 aa, synthetic construct
GTNKRAP

SEQ ID NO: 46 - protein, 9 aa, synthetic construct
ALWYSNLWV

SEQ ID NO: 47 - protein, 125 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSS

SEQ ID NO: 48 - protein, 109 aa, synthetic construct
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 49 - protein, 7 aa, synthetic construct
SNRAAWN

SEQ ID NO: 50 - protein, 18 aa, synthetic construct
RTYYRSKWYNDYAVSVQG

SEQ ID NO: 51 - protein, 9 aa, synthetic construct
VRAVAPFDY

SEQ ID NO: 52 - protein, 17 aa, synthetic construct
KSSQSVLNPSNNKNNLA

SEQ ID NO: 53 - protein, 7 aa, synthetic construct
WASTRES

SEQ ID NO: 54 - protein, 9 aa, synthetic construct
QQYYRTPWT

SEQ ID NO: 55 - protein, 121 aa, synthetic construct
QVQLQQSGPGLLKPSQTLSLTCAISGDSVSSNRAAWNWIRQSPSRGLEWLGRTYYRSKWYNDYAVSVQGRITLIPDTSKNQFSLRLNSVTPEDTAVYYCASVRAVAPFDYWGQGVLVTVSS

SEQ ID NO: 56 - protein, 113 aa, synthetic construct
DIVMTQSPDSLAVSLGERATINCKSSQSVLNPSNNKNNLAWYQQQPGQPPKLLIYWASTRESGVPDRFSGSGSGTDFTLTISSLQAEDVAVYFCQQYYRTPWTFGQGTKVEIK

SEQ ID NO: 57 - protein, 674 aa, synthetic construct
QVQLQQSGPGLLKPSQTLSLTCAISGDSVSSNRAAWNWIRQSPSRGLEWLGRTYYRSKWYNDYAVSVQGRITLIPDTSKNQFSLRLNSVTPEDTAVYYCASVRAVAPFDYWGQGVLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDGGGGSGGGGGQAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSP

SEQ ID NO: 58 - protein, 449 aa, synthetic construct
QVQLQQSGPGLLKPSQTLSLTCAISGDSVSSNRAAWNWIRQSPSRGLEWLGRTYYRSKWYNDYAVSVQGRITLIPDTSKNQFSLRLNSVTPEDTAVYYCASVRAVAPFDYWGQGVLVTVSSASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 59 - protein, 220 aa, synthetic construct
DIVMTQSPDSLAVSLGERATINCKSSQSVLNPSNNKNNLAWYQQQPGQPPKLLIYWASTRESGVPDRFSGSGSGTDFTLTISSLQAEDVAVYFCQQYYRTPWTFGQGTKVEIKRTVAAPSVFIFPPSDRKLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 232 aa, synthetic construct
EVQLLESGGGLVQPGGSLRLSCAASGFTFSSYAMNWVRQAPGKGLEWVSRIRSKYNNYATYYADSVKGRFTISRDDSKNTLYLQMNSLRAEDTAVYYCVRASNFPASYVSYFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 314 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 62 - protein, 446 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 63 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 119 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSS

SEQ ID NO: 65 - protein, 111 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIK

SEQ ID NO: 66 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 67 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 68 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 69 - protein, 497 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 70 - protein, 422 aa, Homo sapiens
VPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHL

SEQ ID NO: 71 - protein, 5 aa, synthetic construct
GGGGS

SEQ ID NO: 72 - protein, 10 aa, synthetic construct
GGGGSGGGGS

SEQ ID NO: 73 - protein, 15 aa, synthetic construct
GGGGSGGGGSGGGGS

SEQ ID NO: 74 - protein, 20 aa, synthetic construct
GGGGSGGGGSGGGGSGGGGS

SEQ ID NO: 75 - protein, 446 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 76 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 77 - protein, 439 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 78 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 79 - protein, 447 aa, synthetic construct
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 80 - protein, 218 aa, synthetic construct
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 119 aa, synthetic construct; identical to SEQ ID NO: 64

SEQ ID NO: 82 - protein, 111 aa, synthetic construct; identical to SEQ ID NO: 65

SEQ ID NO: 83 - protein, 5 aa, synthetic construct
NYYIH

SEQ ID NO: 84 - protein, 17 aa, synthetic construct
WIYPGDGNTKYNEKFKG

SEQ ID NO: 85 - protein, 10 aa, synthetic construct
DSYSNYYFDY

SEQ ID NO: 86 - protein, 17 aa, synthetic construct
KSSQSLLNSRTRKNYLA

SEQ ID NO: 87 - protein, 7 aa, synthetic construct
WASTRES

SEQ ID NO: 88 - protein, 8 aa, synthetic construct
TQSFILRT

SEQ ID NO: 89 - protein, 119 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 90 - protein, 112 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 91 - protein, 7 aa, synthetic construct
SNRAAWN

SEQ ID NO: 92 - protein, 18 aa, synthetic construct
RTYYRSKWYNDYAVSVQG

SEQ ID NO: 93 - protein, 9 aa, synthetic construct
VRAVAPFDY

SEQ ID NO: 94 - protein, 17 aa, synthetic construct
KSSQSVLNSANNKNNLA

SEQ ID NO: 95 - protein, 7 aa, synthetic construct
WASTRES

SEQ ID NO: 96 - protein, 9 aa, synthetic construct
QQYYRTPWT

SEQ ID NO: 97 - protein, 121 aa, synthetic construct; identical to SEQ ID NO: 55

SEQ ID NO: 98 - protein, 113 aa, synthetic construct; identical to SEQ ID NO: 56

SEQ ID NO: 99 - protein, 219 aa, synthetic construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 447 aa, synthetic construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYGSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSP

SEQ ID NO: 101 - protein, 125 aa, synthetic construct
QVQLQQSGPGLVKPSQTLSLTCAISGDSVSSNSAAWNWIRQSPSRGLEWLGRTYYRSKWYNDYAVSVKSRITINPDTSKNQISLQLNSVTPEDTAVYYCAGDRRYGIVGLPFAYWGQGTLVTVSS

SEQ ID NO: 102 - protein, 113 aa, synthetic construct
DIVMTQSPDSLAVSLGERATINCKSSQSVLHSSNNKNYLTWFQQKPGQPPKLLIYWASTRESGVPDRFSGSGSGTDFTLTISSLQAEDVAVYYCHQYYSTPPTFGQGTKVEIK

SEQ ID NO: 103 - protein, 120 aa, synthetic construct
QVQLQQSGPRLVRPSQTLSLTCAISGDSVFNNNAAWSWIRQSPSRGLEWLGRTYYRSKWLYDYAVSVKSRITVNPDTSRNQFTLQLNSVTPEDTALYYCARGYSSSFDYWGQGTLVTVSS

SEQ ID NO: 104 - protein, 110 aa, synthetic construct
QSALTQPASVSGSPGQSITISCTGTSSNIGTYKFVSWYQQHPDKAPKVLLYEVSKRPSGVSSRFSGSKSGNTASLTISGLQAEDQADYHCVSYAGSGTLLFGGGTKLTVL

SEQ ID NO: 105 - protein, 6 aa, synthetic construct
KIEELE

SEQ ID NO: 106 - protein, 440 aa, Homo sapiens
RIIPRHLQLGCGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGCYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWGSGLNDIFEAQKIEWHEHHHHHH

(19) United States
(12) Patent Application Publication
(10) Pub. No.: US 2026/0077044 A1
(43) Pub. Date: Mar. 19, 2026

(54) COMBINATION THERAPY EMPLOYING A PD1-LAG3 BISPECIFIC ANTIBODY AND AN HLA-G T CELL BISPECIFIC ANTIBODY

(71) Applicant: Hoffmann-La Roche Inc., Little Falls, NJ (US)

(72) Inventors: Meher MAJETY, Basel (CH); Carina HAGE, Penzberg (DE); Laura CODARRI DEAK, Au (CH); Valeria Gonzalez NICOLINI, Erlenbach (CH)

(73) Assignee: Hoffmann-La Roche Inc., Little Falls, NJ (US)

(21) Appl. No.: 19/310,373

(22) Filed: Aug. 26, 2025

Related U.S. Application Data
(63) Continuation of application No. PCT/EP2024/056420, filed on Mar. 11, 2024.

(30) Foreign Application Priority Data
Mar. 13, 2023 (EP) 23161501.4

(51) Int. Cl. A61K 39/395; A61K 39/00; A61P 35/00

(52) U.S. Cl. CPC A61K 39/39558; A61P 35/00; A61K 2039/507

(57) ABSTRACT

The invention relates to combination therapies employing an anti-PD1/anti-LAG3 bispecific antibody and an HLA-G T cell-activating bispecific antibody, the use of these combination therapies for the treatment of cancer and methods of using the combination therapies.

### CROSS-REFERENCE TO RELATED APPLICATIONS

This application is a continuation of International Patent Application Serial No. PCT/EP2024/056420, filed Mar. 11, 2024, which claims the benefit of priority to EP Application Serial No. 23161501.4, filed Mar. 13, 2023, all of which are incorporated herein by reference in their entirety and for all purposes.

### SEQUENCE LISTING

This application contains a Sequence Listing which has been submitted electronically in XML format and is hereby incorporated by reference in its entirety. Said XML copy, created on Aug. 18, 2025, is named “P38325-US-1-SEQUENCE_LISTING.xml” and is 105,383 bytes in size.

### FIELD OF THE INVENTION

The present invention relates to combination therapies employing a PD1-LAG3 bispecific antibody and an HLA-G T cell-activating bispecific antibody, the use of these combination therapies for the treatment of cancer and methods of using the combination therapies.

### BACKGROUND

T cell activating bispecific antibodies are a promising class of cancer therapeutics, designed to engage cytotoxic T cells against tumor cells. The simultaneous binding of such an antibody to CD3 on T cells and to an antigen expressed on the tumor cells leads to activation of the T cell and subsequent lysis of the tumor cell.

The human major histocompatibility complex, class I, 6, also known as human leukocyte antigen G (HLA-G), is a protein that in humans is encoded by the HLA-G gene. HLA-G belongs to the HLA nonclassical class I heavy chain paralogues. This class I molecule is a heterodimer consisting of a heavy chain and a light chain (beta-2 microglobulin). The heavy chain is anchored in the membrane but can also be shedded/secreted. HLA-G is predominantly expressed on cytotrophoblasts in the placenta. Several tumors (including pancreatic, breast, skin, colorectal, gastric & ovarian) express HLA-G (Lin, A. et al., Mol Med. 21 (2015) 782-791; Amiot, L., et al., Cell Mol Life Sci. 68 (2011) 417-431). Expression has also been reported to be associated with pathological conditions like inflammatory diseases, GvHD and cancer. Expression of HLA-G has been reported to be associated with poor prognosis in cancer. Tumor cells escape host immune surveillance by inducing immune tolerance/suppression via HLA-G expression.

HLA-G shares high homology (>98%) with other MHC I molecules, therefore truly HLA-G specific antibodies with no crossreactivity to other MHC I molecules are difficult to generate. Due to the high polymorphism and high homology of the HLA family many antibodies lack either truly specific HLA-G binding properties and often also bind or crossreact with other HLA family members (either as MHC complex with β2M or in its B2M-free form) or they simply do not inhibit binding of HLA-Gβ2M MHC complex to its receptors ILT2 and/or ILT4 (and are regarded as non-antagonistic antibodies). Antibodies specifically binding to HLA-G are described in WO 2019/202040, WO 2019/202041 and WO 2022129120. These documents also describe T cell bispecific antibodies comprising a binding moiety specifically binding to HLA-G.

Anti-tumor activity of HLA-G-TCB has been demonstrated in vitro using different HLA-G positive tumor cell lines, and in vivo, using mouse models. T cell activation, IFNγ secretion, and cytotoxicity induced by HLA-G-TCB were shown to be dose dependent and correlating to the HLA-G density on cell surface and the percentage of HLA G positive cells. In line with the mode of action of TCBs, tumor growth inhibition was accompanied by increased cytokine secretion (including IFNγ), tumoral T cell infiltration and activation as reflected by increased expression of T cell activation markers including CD69, CD25 and Granzyme B. Evaluation of HLA-G expression on tumor cell lines upon IFNγ stimulation in vitro and in PDX (patient derived xenograft) tumors treated with HLA-G TCB revealed that HLA-G expression can be upregulated upon IFNγ stimulation and TCB treatment respectively and may positively impact the anti-tumor activity of HLA-G-TCB in vivo.

T cell exhaustion can pose a challenge if the redirected T cells are not able to effectively recognize and destroy the targeted tumor cells, resulting in a decreased efficacy of the T cell directed immunotherapy. Exhausted T cells are characterized by sustained expression of the inhibitory molecule PD1 (programmed cell death protein 1) and it has been found that blockade of PD1 and PD-L1 (PD-1 ligand) interactions can reverse T cell exhaustion and restore antigen-specific T cell responses. However, targeting the PD1-PD-L1 pathway alone does not always result in reversal of T cell exhaustion, possibly due to resistance mechanisms, immunosuppressive activity of MDSC, and/or regulatory T cells.

Lymphocyte activation gene-3 (LAG3 or CD223) was initially discovered in an experiment designed to selectively isolate molecules expressed in an IL-2-dependent NK cell line (Triebel F et al., Cancer Lett. 235 (2006), 147-153). LAG3 is a unique transmembrane protein with structural homology to CD4 with four extracellular immunoglobulin superfamily-like domains (D1-D4). The membrane-distal IgG domain contains a short amino acid sequence, the so-called extra loop that is not found in other IgG superfamily proteins. The intracellular domain contains a unique amino acid sequence (KIEELE, SEQ ID NO: 105) that is required for LAG3 to exert a negative effect on T cell function. LAG3 can be cleaved at the connecting peptide (CP) by metalloproteases to generate a soluble form, which is detectable in serum. Like CD4, the LAG3 protein binds to MHC class II molecules, however with a higher affinity and at a distinct site from CD4 (Huard et al. Proc. Natl. Acad. Sci. USA 94 (1997), 5744-5749). LAG3 is expressed by T cells, B cells, NK cells and plasmacytoid dendritic cells (pDCs) and is upregulated following T cell activation. It modulates T cell function as well as T cell homeostasis. Subsets of conventional T cells that are anergic or display impaired functions express LAG3. LAG3⁺ T cells are enriched at tumor sites and during chronic viral infections (Sierro et al Expert Opin. Ther. Targets 15 (2011), 91-101). It has been shown that LAG3 plays a role in CD8 T cell exhaustion (Blackburn et al. Nature Immunol. 10 (2009), 29-37). Thus, there is a need for antibodies that antagonize the activity of LAG3 and can be used to generate and restore immune response to tumors.

By targeting both PD1 and LAG3 on dysfunctional tumor-specific T lymphocytes, PD1-LAG3 aims to restore an effective anti-tumor immune-response and to provide survival benefit to more cancer patients than the currently available checkpoint inhibitors. By preferentially targeting PD1/LAG3 co-expressing dysfunctional T cells and potentially reduced targeting of LAG3 expressing Tregs in the tumor microenvironment, PD1-LAG3 BsAb might avoid reinvigorating Treg mediated immunosuppressive effects while restoring the anti-tumor immune response.

There is a need for new combination treatments using two or more bispecific antibodies in order to improve immunotherapy by additive stimulatory effects that cannot be achieved with a single molecule in order to overcome the effects caused by the lack of co-stimulation during T cell bispecific antibody-mediated cancer immunotherapy such as poor persistence of T cell responses.

### SUMMARY OF THE INVENTION

The present invention relates to combination therapies employing an anti-HLA-G/anti-CD3 bispecific antibody and a bispecific antibody comprising a first antigen binding domain that specifically binds to programmed cell death protein 1 (PD1) and a second antigen binding domain that specifically binds to Lymphocyte activation gene-3 (LAG3). It has been found, that the anti-PD1/anti-LAG3 bispecific antibodies as described herein are advantageous over anti-PD1 antibodies, in particular in combination with an anti-HLA-G/anti-CD3 bispecific antibody, as they provide better selectivity and efficacy. These anti-PD1/anti-LAG3 bispecific antibodies are further characterized in that they show a reduced sink effect (as shown by reduced internalization by T cells), they preferentially bind to conventional T cells as to Tregs and are able to rescue T cell effector functions from Treg suppression, they show increased tumor-specific T cell effector functions and increased tumor eradication in vivo. Based on these properties they are advantageous to be used in combination with T cell bispecific antibodies, in particular anti-HLA-G/anti-CD3 bispecific antibodies.

Described herein is an anti-HLA-G/anti-CD3 bispecific antibody for use in a method of treating cancer, in particular HLA-G expressing cancer, wherein the anti-HLA-G/anti-CD3 bispecific antibody is used in combination with an anti-PD1/anti-LAG3 bispecific antibody.

The invention provides an anti-HLA-G/anti-CD3 bispecific antibody for use in a method as defined hereinbefore, wherein the anti-PD1/anti-LAG3 bispecific antibody comprises a first antigen binding domain that specifically binds to programmed cell death protein 1 (PD1) and a second antigen binding domain that specifically binds to Lymphocyte activation gene-3 (LAG3), wherein the first antigen binding domain specifically binding to PD1 comprises a VH domain comprising
(i) CDR-H1 comprising the amino acid sequence of SEQ ID NO:1.
(ii) CDR-H2 comprising the amino acid sequence of SEQ ID NO:2, and
(iii) CDR-H3 comprising the amino acid sequence of SEQ ID NO:3; and
   a VL domain comprising
(i) CDR-L1 comprising the amino acid sequence of SEQ ID NO:4;
(ii) CDR-L2 comprising the amino acid sequence of SEQ ID NO:5, and
(iii) CDR-L3 comprising the amino acid sequence of SEQ ID NO:6.

In one aspect, an anti-HLA-G/anti-CD3 bispecific antibody is provided for use in a method of treating cancer, in particular HLA-G expressing cancer, wherein the anti-HLA-G/anti-CD3 bispecific antibody and the anti-PD1/anti-LAG3 bispecific antibody are administered together in a single composition or administered separately in two or more different compositions.

Furthermore, provided is an anti-HLA-G/anti-CD3 bispecific antibody for use in a method of treating cancer, in particular HLA-G expressing cancer, wherein the anti-HLA-G/anti-CD3 bispecific antibody is used in combination with an anti-PD1/anti-LAG3 bispecific antibody and wherein the anti-PD1/anti-LAG3 bispecific antibody comprises a Fc domain that is an IgG Fc domain, particularly an IgG1 Fc domain or an IgG4 Fc domain, and wherein the Fc domain comprises one or more amino acid substitutions that reduce binding to an Fc receptor, in particular towards Fcγ receptor. More particularly, the anti-PD1/anti-LAG3 bispecific antibody comprises an Fc domain of human IgG1 subclass with the amino acid mutations L234A, L235A and P329G (numbering according to Kabat EU index).

In one aspect, provided is an anti-HLA-G/anti-CD3 bispecific antibody for use in a method as described herein before, wherein the anti-PD1/anti-LAG3 bispecific antibody comprises a second antigen binding domain that specifically binds to LAG3 comprising
(a) a VH domain comprising
   (i) CDR-H1 comprising the amino acid sequence of SEQ ID NO:11,
   (ii) CDR-H2 comprising the amino acid sequence of SEQ ID NO:12, and
   (iii) CDR-H3 comprising the amino acid sequence of SEQ ID NO:13; and
   a VL domain comprising
   (i) CDR-L1 comprising the amino acid sequence of SEQ ID NO:14,
   (ii) CDR-L2 comprising the amino acid sequence of SEQ ID NO:15, and
   (iii) CDR-L3 comprising the amino acid sequence of SEQ ID NO:16; or
(b) a VH domain comprising
   (i) CDR-H1 comprising the amino acid sequence of SEQ ID NO:19,
   (ii) CDR-H2 comprising the amino acid sequence of SEQ ID NO:20, and
   (iii) CDR-H3 comprising the amino acid sequence of SEQ ID NO:21; and
   a VL domain comprising
   (i) CDR-L1 comprising the amino acid sequence of SEQ ID NO:22,
   (ii) CDR-L2 comprising the amino acid sequence of SEQ ID NO:23, and
   (iii) CDR-L3 comprising the amino acid sequence of SEQ ID NO:24.

In another aspect, provided is an anti-HLA-G/anti-CD3 bispecific antibody for use in a method as disclosed herein, wherein the anti-PD1/anti-LAG3 bispecific antibody comprises a first antigen-binding domain specifically binding to PD1 comprising a VH domain comprising the amino acid sequence of SEQ ID NO:9 and a VL domain comprising the amino acid sequence of SEQ ID NO:10.

In a further aspect, provided is an anti-HLA-G/anti-CD3 bispecific antibody for use as described herein, wherein the anti-PD1/anti-LAG3 bispecific antibody comprises a second antigen-binding domain specifically binding to LAG3 comprising
(a) a VH domain comprising the amino acid sequence of SEQ ID NO:17 and a VL domain comprising the amino acid sequence of SEQ ID NO:18, or
(b) a VH domain comprising the amino acid sequence of SEQ ID NO:25 and a VL domain comprising the amino acid sequence of SEQ ID NO:26.

In an additional aspect, provided is an anti-HLA-G/anti-CD3 bispecific antibody for use in a method as described herein, wherein the anti-PD1/anti-LAG3 bispecific antibody comprises a second antigen-binding domain specifically binding to LAG3 comprising
(a) a VH domain comprising the amino acid sequence of SEQ ID NO:27 and a VL domain comprising the amino acid sequence of SEQ ID NO:28, or
(b) a VH domain comprising the amino acid sequence of SEQ ID NO:29 and a VL domain comprising the amino acid sequence of SEQ ID NO:30, or
(c) a VH domain comprising the amino acid sequence of SEQ ID NO:31 and a VL domain comprising the amino acid sequence of SEQ ID NO:32, or
(d) a VH domain comprising the amino acid sequence of SEQ ID NO:33 and a VL domain comprising the amino acid sequence of SEQ ID NO:34, or
(e) a VH domain comprising the amino acid sequence of SEQ ID NO:64 and a VL domain comprising the amino acid sequence of SEQ ID NO:65.

Furthermore, provided is an anti-HLA-G/anti-CD3 bispecific antibody for use in a method as disclosed herein, wherein the anti-PD1/anti-LAG3 bispecific antibody comprises a first antigen binding domain specifically binding to PD1 comprising a VH domain comprising the amino acid sequence of SEQ ID NO:9 and a VL domain comprising the amino acid sequence of SEQ ID NO:10, and a second antigen binding domain specifically binding to LAG3 comprising a VH domain comprising the amino acid sequence of SEQ ID NO:17 and a VL domain comprising the amino acid sequence of SEQ ID NO:18.

In a further aspect, provided is an anti-HLA-G/anti-CD3 bispecific antibody for use in a method of treating cancer, in particular HLA-G expressing cancer, wherein the anti-PD1/anti-LAG3 bispecific antibody comprises a Fab fragment specifically binding to PD1 and a Fab fragment specifically binding to LAG3. In one aspect, the anti-PD1/anti-LAG3 bispecific antibody comprises a Fab fragment specifically binding to PD1, wherein the variable domains VL and VH are replaced by each other so that VL is part of the heavy chain and VH is part of the light chain.

In another aspect, provided is an anti-HLA-G/anti-CD3 bispecific antibody for use in a method as disclosed herein before, wherein the anti-PD1/anti-LAG3 bispecific antibody comprises monovalent binding to PD-1 and monovalent binding to LAG3.

In a further aspect, provided is an anti-HLA-G/anti-CD3 bispecific antibody for use in a method as disclosed herein before, wherein the anti-PD1/anti-LAG3 bispecific antibody is a humanized or chimeric antibody. In particular, the anti-PD1/anti-LAG3 bispecific antibody is a humanized antibody. Furthermore, provided is an anti-PD1/anti-LAG3 bispecific antibody as described herein before, wherein the anti-PD1/anti-LAG3 bispecific antibody comprises an Fc domain comprising a modification promoting the association of the first and second subunit of the Fc domain. In one aspect, provided is an anti-PD1/anti-LAG3 bispecific antibody, wherein the first subunit of the Fc domain comprises knobs and the second subunit of the Fc domain comprises holes according to the knobs into holes method. In particular, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

In a particular aspect, provided is a anti-HLA-G/anti-CD3 bispecific antibody for use in a method of treating cancer, in particular HLA-G expressing cancer, wherein the anti-PD1/anti-LAG3 bispecific antibody comprises
(a) a first heavy chain comprising an amino acid sequence of SEQ ID NO:35, a first light chain comprising an amino acid sequence of SEQ ID NO:36, a second heavy chain comprising an amino acid sequence of SEQ ID NO:37, and a second light chain comprising an amino acid sequence of SEQ ID NO:38, or
(b) a first heavy chain comprising an amino acid sequence of SEQ ID NO:35, a first light chain comprising an amino acid sequence of SEQ ID NO:36, a second heavy chain comprising an amino acid sequence of SEQ ID NO:39, and a second light chain comprising an amino acid sequence of SEQ ID NO:40.

More particularly, the anti-PD1/anti-LAG3 bispecific antibody comprises a first heavy chain comprising an amino acid sequence of SEQ ID NO:35, a first light chain comprising an amino acid sequence of SEQ ID NO:36, a second heavy chain comprising an amino acid sequence of SEQ ID NO:37, and a second light chain comprising an amino acid sequence of SEQ ID NO:38.

Furthermore, provided is an anti-HLA-G/anti-CD3 bispecific antibody for use in a method of treating cancer, in particular HLA-G expressing cancer, wherein the anti-HLA-G/anti-CD3 bispecific antibody is for use in combination with an anti-PD1/anti-LAG3 bispecific antibody and wherein the anti-HLA-G/anti-CD3 bispecific antibody comprises a first antigen binding domain specifically binding to CD3 comprising a heavy chain variable region (V_{H}CD3) and a light chain variable region (V_{L}CD3), and a second antigen binding domain specifically binding to HLA-G comprising a heavy chain variable region (V_{H}HLA-G) and a light chain variable region (V_{L}HLA-G).

In one aspect, the anti-HLA-G/anti-CD3 bispecific antibody comprises a first antigen binding domain specifically binding to CD3 comprising
a heavy chain variable region (V_{H}CD3) comprising CDR-H1 sequence of SEQ ID NO:41, CDR-H2 sequence of SEQ ID NO:42, and CDR-H3 sequence of SEQ ID NO:43; and/or
a light chain variable region (V_{L}CD3) comprising CDR-L1 sequence of SEQ ID NO:44, CDR-L2 sequence of SEQ ID NO:45, and CDR-L3 sequence of SEQ ID NO:46.

More particularly, the anti-HLA-G/anti-CD3 bispecific antibody comprises a first antigen binding domain specifically binding to CD3 comprising a heavy chain variable region (V_{H}CD3) comprising the amino acid sequence of SEQ ID NO:47 and/or a light chain variable region (V_{L}CD3) comprising the amino acid sequence of SEQ ID NO:48.

In one aspect, the anti-HLA-G/anti-CD3 bispecific antibody for use in a method of treating cancer, in particular HLA-G expressing cancer, comprises a second antigen binding domain specifically binding to HLA-G comprising
a heavy chain variable region (V_{H}HLA-G) comprising CDR-H1 sequence of SEQ ID NO:49, CDR-H2 sequence of SEQ ID NO:50, and CDR-H3 sequence of SEQ ID NO:51, and/or
a light chain variable region (V_{L}HLA-G) comprising CDR-L1 sequence of SEQ ID NO:52, CDR-L2 sequence of SEQ ID NO:53, and CDR-L3 sequence of SEQ ID NO:54.

In particular, the second antigen binding domain comprises a heavy chain variable region (V_{H}HLA-G) comprising the amino acid sequence of SEQ ID NO:55 and/or a light chain variable region (VI HLA-G) comprising the amino acid sequence of SEQ ID NO:56. In a further aspect, the anti-HLA-G/anti-CD3 bispecific antibody for use in a method of treating cancer, in particular HLA-G expressing cancer, comprises a third antigen binding domain that binds to HLA-G. In another aspect, the anti-HLA-G/anti-CD3 bispecific antibody comprises an Fc domain comprising one or more amino acid substitutions that reduce binding to an Fc receptor and/or effector function.

In one aspect, provided is the method wherein the anti-HLA-G/anti-CD3 bispecific antibody comprises a first Fab fragment specifically binding to CD3 comprising a VH domain comprising the amino acid sequence of SEQ ID NO:47 and a VL domain comprising the amino acid sequence of SEQ ID NO:48, and a second, and optionally a third, Fab fragment specifically binding to HLA-G comprising a VH domain comprising the amino acid sequence of SEQ ID NO:55 and a VL domain comprising the amino acid sequence of SEQ ID NO:56.

In a further aspect, provided is an anti-HLA-G/anti-CD3 bispecific antibody for use in a method of treating cancer, in particular an HLA-G expressing cancer, wherein the anti-HLA-G/anti-CD3 bispecific antibody is used in combination with an anti-PD1/anti-LAG3 bispecific antibody and wherein the combination is for administration at intervals from about one week to three weeks.

In one further aspect, provided is a composition comprising an anti-PD1/anti-LAG3 bispecific antibody for use in the treatment of cancer, in particular HLA-G expressing cancer, wherein said treatment comprises administration of said composition comprising an anti-PD1/anti-LAG3 bispecific antibody in combination with a composition comprising an anti-HLA-G/anti-CD3 bispecific antibody, wherein the anti-PD1/anti-LAG3 bispecific antibody comprises a first antigen binding domain that specifically binds to programmed cell death protein 1 (PD1) and a second antigen binding domain that specifically binds to Lymphocyte activation gene-3 (LAG3), wherein the first antigen binding domain specifically binding to PD1 comprises a VH domain comprising
(i) CDR-H1 comprising the amino acid sequence of SEQ ID NO:1,
(ii) CDR-H2 comprising the amino acid sequence of SEQ ID NO:2, and
(iii) CDR-H3 comprising the amino acid sequence of SEQ ID NO:3; and
a VL domain comprising
(i) CDR-L1 comprising the amino acid sequence of SEQ ID NO:4;
(ii) CDR-L2 comprising the amino acid sequence of SEQ ID NO:5, and
(iii) CDR-L3 comprising the amino acid sequence of SEQ ID NO:6.

In one aspect, the composition comprises an anti-PD1/anti-LAG3 bispecific antibody comprising a first antigen-binding domain specifically binding to PD1 comprising the VH domain comprising the amino acid sequence of SEQ ID NO:9 and the VL domain comprising the amino acid sequence of SEQ ID NO:10.

In one further aspect, the composition comprises an anti-PD1/anti-LAG3 bispecific antibody comprising a second antigen binding domain that specifically binds to LAG3 comprising
(a) a VH domain comprising
   (i) CDR-H1 comprising the amino acid sequence of SEQ ID NO:11,
   (ii) CDR-H2 comprising the amino acid sequence of SEQ ID NO:12, and
   (iii) CDR-H3 comprising the amino acid sequence of SEQ ID NO:13; and
   a VL domain comprising
   (i) CDR-L1 comprising the amino acid sequence of SEQ ID NO:14,
   (ii) CDR-L2 comprising the amino acid sequence of SEQ ID NO:15, and
   (iii) CDR-L3 comprising the amino acid sequence of SEQ ID NO:16; or
(b) a VH domain comprising
   (i) CDR-H1 comprising the amino acid sequence of SEQ ID NO:19,
   (ii) CDR-H2 comprising the amino acid sequence of SEQ ID NO:20, and
   (iii) CDR-H3 comprising the amino acid sequence of SEQ ID NO:21; and
   a VL domain comprising
   i) CDR-L1 comprising the amino acid sequence of SEQ ID NO:22,
   (ii) CDR-L2 comprising the amino acid sequence of SEQ ID NO:23, and
   (iii) CDR-L3 comprising the amino acid sequence of SEQ ID NO:24.

In one aspect, the composition comprises an anti-PD1/anti-LAG3 bispecific antibody comprising an antigen-binding domain specifically binding to LAG3 comprising
(a) a VH domain comprising the amino acid sequence of SEQ ID NO:17 and a VL domain comprising the amino acid sequence of SEQ ID NO:18, or
(b) a VH domain comprising the amino acid sequence of SEQ ID NO:25 and a VL domain comprising the amino acid sequence of SEQ ID NO:26.

In one particular aspect, the composition comprises an anti-PD1/anti-LAG3 bispecific antibody comprising
a first Fab fragment specifically binding to PD1 comprising a VH domain comprising the amino acid sequence of SEQ ID NO:9 and a VL domain comprising the amino acid sequence of SEQ ID NO:10, and
a second Fab fragment specifically binding to LAG3 comprising a VH domain comprising the amino acid sequence of SEQ ID NO: 17 and a VL domain comprising the amino acid sequence of SEQ ID NO:18.

Furthermore, provided is a composition comprising an anti-PD1/anti-LAG3 bispecific antibody for use in the treatment of cancer, in particular an HLA-G expressing cancer, wherein said treatment comprises administration of said composition comprising an anti-PD1/anti-LAG3 bispecific antibody in combination with a composition comprising an anti-HLA-G/anti-CD3 bispecific antibody, wherein the anti-HLA-G/anti-CD3 bispecific antibody comprises a first antigen binding domain comprising a heavy chain variable region (V_{H}CD3) and a light chain variable region (V_{L}CD3), and a second antigen binding domain comprising a heavy chain variable region (V_{H}HLA-G) and a light chain variable region (V_{L}HLA-G).

In one aspect, the anti-HLA-G/anti-CD3 bispecific antibody comprises a first antigen binding domain comprising a heavy chain variable region (V_{H}CD3) comprising CDR-H1 sequence of SEQ ID NO:41, CDR-H2 sequence of SEQ ID NO:42, and CDR-H3 sequence of SEQ ID NO:43; and/or a light chain variable region (V_{L}CD3) comprising CDR-L1 sequence of SEQ ID NO:44, CDR-L2 sequence of SEQ ID NO:45, and CDR-L3 sequence of SEQ ID NO:46. More particularly, the anti-HLA-G/anti-CD3 bispecific antibody comprises a first antigen binding domain comprising a heavy chain variable region (V_{H}CD3) comprising the amino acid sequence of SEQ ID NO: 47 and/or a light chain variable region (V_{L}CD3) comprising the amino acid sequence of SEQ ID NO:48. In one aspect, the anti-HLA-G/anti-CD3 bispecific antibody comprises a second antigen binding domain comprising a heavy chain variable region (V_{H}HLA-G) comprising CDR-H1 sequence of SEQ ID NO:49, CDR-H2 sequence of SEQ ID NO:50, and CDR-H3 sequence of SEQ ID NO:51, and/or a light chain variable region (V_{L}HLA-G) comprising CDR-L1 sequence of SEQ ID NO:52, CDR-L2 sequence of SEQ ID NO:53, and CDR-L3 sequence of SEQ ID NO:54. In particular, the second antigen binding domain comprises a heavy chain variable region (V_{H}HLA-G) comprising the amino acid sequence of SEQ ID NO:55 and/or a light chain variable region (VI HLA-G) comprising the amino acid sequence of SEQ ID NO:56. In a further aspect, the anti-HLA-G/anti-CD3 bispecific antibody comprises a third antigen binding domain that binds to HLA-G. In particular, the first antigen binding domain specifically binding to CD3 comprises a VH domain comprising the amino acid sequence of SEQ ID NO:47 and a VL domain comprising the amino acid sequence of SEQ ID NO:48, and the second (and optionally third) antigen binding domain specifically binding to HLA-G comprises a VH domain comprising the amino acid sequence of SEQ ID NO:55 and a VL domain comprising the amino acid sequence of SEQ ID NO: 56. In another aspect, the anti-HLA-G/anti-CD3 bispecific antibody comprises an Fc domain comprising one or more amino acid substitutions that reduce binding to an Fc receptor and/or effector function.

In a further aspect, provided is a pharmaceutical product comprising
(A) a first composition comprising as active ingredient an anti-HLA-G/anti-CD3 bispecific antibody and a pharmaceutically acceptable carrier; and
(B) a second composition comprising as active ingredient an anti-PD1/anti-LAG3 bispecific antibody and a pharmaceutically acceptable carrier,
for use in the combined, sequential or simultaneous, treatment of a disease, in particular cancer, particularly HLA-G expressing cancer.

In another aspect, provided is a pharmaceutical composition comprising a combination of an anti-HLA-G/anti-CD3 bispecific antibody and an anti-PD1/anti-LAG3 bispecific antibody for use in the combined, sequential or simultaneous, treatment of a disease, in particular cancer, particularly HLA-G expressing cancer, particularly a disease selected from lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, carcinoma of the fallopian tubes, carcinoma of the endometrium, carcinoma of the cervix, carcinoma of the vagina, carcinoma of the vulva, Hodgkin's Disease, nasopharyngeal carcinoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymomas, medulloblastomas, meningiomas, squamous cell carcinomas, pituitary adenoma, lymphoma, lymphocytic leukemia, including refractory versions of any of the above cancers, or a combination of one or more of the above cancers. In particular, the pharmaceutical composition is for use in the treatment of solid tumors including but not limited to renal cell carcinoma, colorectal carcinoma, non-small cell lung cancer and pancreatic duct adenocarcinoma (PDAC).

In another aspect, provided is the use of a combination of an anti-HLA-G/anti-CD3 bispecific antibody and an anti-PD1/anti-LAG3 bispecific antibody in the manufacture of a medicament for treating or delaying progression of a proliferative disease, in particular cancer, particularly an HLA-G expressing cancer, wherein the anti-PD1/anti-LAG3 bispecific antibody comprises a first antigen binding domain that specifically binds to programmed cell death protein 1 (PD1) and a second antigen binding domain that specifically binds to Lymphocyte activation gene-3 (LAG3), wherein the first antigen binding domain specifically binding to PD1 comprises a VH domain comprising
(i) CDR-H1 comprising the amino acid sequence of SEQ ID NO:1,
(ii) CDR-H2 comprising the amino acid sequence of SEQ ID NO:2, and
(iii) CDR-H3 comprising the amino acid sequence of SEQ ID NO:3; and
a VL domain comprising
(i) CDR-L1 comprising the amino acid sequence of SEQ ID NO:4;
(ii) CDR-L2 comprising the amino acid sequence of SEQ ID NO:5, and
(iii) CDR-L3 comprising the amino acid sequence of SEQ ID NO:6.

In one further aspect, the anti-PD1/anti-LAG3 bispecific antibody comprises a second antigen binding domain that specifically binds to LAG3 comprising
(a) a VH domain comprising
(i) CDR-H1 comprising the amino acid sequence of SEQ ID NO:11,
(ii) CDR-H2 comprising the amino acid sequence of SEQ ID NO:12, and
(iii) CDR-H3 comprising the amino acid sequence of SEQ ID NO:13; and
a VL domain comprising
(i) CDR-L1 comprising the amino acid sequence of SEQ ID NO:14,
(ii) CDR-L2 comprising the amino acid sequence of SEQ ID NO:15, and
(iii) CDR-L3 comprising the amino acid sequence of SEQ ID NO:16; or
(b) a VH domain comprising
(i) CDR-H1 comprising the amino acid sequence of SEQ ID NO:19.
(ii) CDR-H2 comprising the amino acid sequence of SEQ ID NO:20, and
(iii) CDR-H3 comprising the amino acid sequence of SEQ ID NO:21; and
a VL domain comprising
(i) CDR-L1 comprising the amino acid sequence of SEQ ID NO:22,
(ii) CDR-L2 comprising the amino acid sequence of SEQ ID NO:23, and
(iii) CDR-L3 comprising the amino acid sequence of SEQ ID NO:24.

In another aspect, provided is the use of a combination of an anti-HLA-G/anti-CD3 bispecific antibody and an anti-PD1/anti-LAG3 bispecific antibody in the manufacture of a medicament for treating or delaying progression of a proliferative disease, in particular cancer, particularly an HLA-G expressing cancer, wherein the anti-PD1/anti-LAG3 bispecific antibody comprises a first Fab fragment specifically binding to PD1 comprising a VH domain comprising the amino acid sequence of SEQ ID NO:9 and a VL domain comprising the amino acid sequence of SEQ ID NO:10, and a second Fab fragment specifically binding to LAG3 comprising a VH domain comprising the amino acid sequence of SEQ ID NO: 17 and a VL domain comprising the amino acid sequence of SEQ ID NO:18.

In a further aspect, provided is a method for treating cancer, in particular HLA-G expressing cancer, in a subject comprising administering to the subject an effective amount of an anti-HLA-G/anti-CD3 antibody and an effective amount of an anti-PD1/anti-LAG3 bispecific antibody, wherein the anti-PD1/anti-LAG3 bispecific antibody comprises a first antigen binding domain that specifically binds to programmed cell death protein 1 (PD1) and a second antigen binding domain that specifically binds to Lymphocyte activation gene-3 (LAG3), wherein the first antigen binding domain specifically binding to PD1
comprises a VH domain comprising
(i) CDR-H1 comprising the amino acid sequence of SEQ ID NO:1,
(ii) CDR-H2 comprising the amino acid sequence of SEQ ID NO:2, and
(iii) CDR-H3 comprising the amino acid sequence of SEQ ID NO:3; and
   a VL domain comprising
(i) CDR-L1 comprising the amino acid sequence of SEQ ID NO:4;
(ii) CDR-L2 comprising the amino acid sequence of SEQ ID NO:5, and
(iii) CDR-L3 comprising the amino acid sequence of SEQ ID NO:6.

In one aspect, provided is the method, wherein the anti-PD1/anti-LAG3 bispecific antibody comprises a second antigen binding domain that specifically binds to LAG3 comprising
(a) a VH domain comprising
(i) CDR-H1 comprising the amino acid sequence of SEQ ID NO:11,
(ii) CDR-H2 comprising the amino acid sequence of SEQ ID NO:12, and
(iii) CDR-H3 comprising the amino acid sequence of SEQ ID NO:13; and
a VL domain comprising
(i) CDR-L1 comprising the amino acid sequence of SEQ ID NO:14,
(ii) CDR-L2 comprising the amino acid sequence of SEQ ID NO:15, and
(iii) CDR-L3 comprising the amino acid sequence of SEQ ID NO:16; or
(b) a VH domain comprising
(i) CDR-H1 comprising the amino acid sequence of SEQ ID NO:19,
(ii) CDR-H2 comprising the amino acid sequence of SEQ ID NO:20, and
(iii) CDR-H3 comprising the amino acid sequence of SEQ ID NO:21; and
a VL domain comprising
(i) CDR-L1 comprising the amino acid sequence of SEQ ID NO:22,
(ii) CDR-L2 comprising the amino acid sequence of SEQ ID NO:23, and
(iii) CDR-L3 comprising the amino acid sequence of SEQ ID NO:24.

In another aspect, provided is the method for treating cancer wherein the anti-PD1/anti-LAG3 bispecific antibody comprises a first Fab fragment specifically binding to PD1 comprising a VH domain comprising the amino acid sequence of SEQ ID NO:9 and a VL domain comprising the amino acid sequence of SEQ ID NO:10, and a second Fab fragment specifically binding to LAG3 comprising a VH domain comprising the amino acid sequence of SEQ ID NO: 17 and a VL domain comprising the amino acid sequence of SEQ ID NO:18.

In one aspect, provided is the method for treating cancer, wherein the anti-HLA-G/anti-CD3 bispecific antibody comprises a first antigen binding domain specifically binding to CD3 comprising a heavy chain variable region (V_{H}CD3) and a light chain variable region (V_{L}CD3), and a second antigen binding domain specifically binding to HLA-G comprising a heavy chain variable region (V_{H}HLA-G) and a light chain variable region (V_{L}HLA-G). In one aspect, the anti-HLA-G/anti-CD3 bispecific antibody comprises a first antigen binding domain comprising a heavy chain variable region (V_{H}CD3) comprising CDR-H1 sequence of SEQ ID NO:41. CDR-H2 sequence of SEQ ID NO:42, and CDR-H3 sequence of SEQ ID NO:43; and/or a light chain variable region (V_{L}CD3) comprising CDR-L1 sequence of SEQ ID NO:44, CDR-L2 sequence of SEQ ID NO:45, and CDR-L3 sequence of SEQ ID NO:46. More particularly, the anti-HLA-G/anti-CD3 bispecific antibody comprises a first antigen binding domain comprising a heavy chain variable region (V_{H}CD3) comprising the amino acid sequence of SEQ ID NO:47 and/or a light chain variable region (V_{L}CD3) comprising the amino acid sequence of SEQ ID NO:48. In one aspect, the anti-HLA-G/anti-CD3 bispecific antibody comprises a second antigen binding domain comprising a heavy chain variable region (V_{H}HLA-G) comprising CDR-H1 sequence of SEQ ID NO:49, CDR-H2 sequence of SEQ ID NO:50, and CDR-H3 sequence of SEQ ID NO:51, and/or a light chain variable region (V_{L}HLA-G) comprising CDR-L1 sequence of SEQ ID NO:52. CDR-L2 sequence of SEQ ID NO:53, and CDR-L3 sequence of SEQ ID NO:54. In particular, the second antigen binding domain comprises a heavy chain variable region (V_{H}HLA-G) comprising the amino acid sequence of SEQ ID NO:55 and/or a light chain variable region (V_{L}HLA-G) comprising the amino acid sequence of SEQ ID NO:56. In a further aspect, the anti-HLA-G/anti-CD3 bispecific antibody comprises a third antigen binding domain that binds to HLA-G. In particular, the first antigen binding domain specifically binding to CD3 comprises a VH domain comprising the amino acid sequence of SEQ ID NO:47 and a VL domain comprising the amino acid sequence of SEQ ID NO: 48, and the second (and optionally third) antigen binding domain specifically binding to HLA-G comprises a VH domain comprising the amino acid sequence of SEQ ID NO:55 and a VL domain comprising the amino acid sequence of SEQ ID NO:56. In another aspect, the anti-HLA-G/anti-CD3 bispecific antibody comprises an Fc domain comprising one or more amino acid substitutions that reduce binding to an Fc receptor and/or effector function.

In one aspect, the anti-HLA-G/anti-CD3 bispecific antibody and the anti-PD1/anti-LAG3 bispecific antibody are administered together in a single composition or administered separately in two or more different compositions. In a further aspect, the anti-HLA-G/anti-CD3 bispecific antibody and the anti-PD1/anti-LAG3 bispecific antibody are administered intravenously or subcutaneously. In another aspect, the anti-HLA-G/anti-CD3 bispecific antibody is administered concurrently with, prior to, or subsequently to the anti-PD1/anti-LAG3 bispecific antibody.

In any of the above aspects the subject is preferably a mammal, particularly a human.

### BRIEF DESCRIPTION OF THE DRAWINGS

FIGS. 1A and 1B are schematic illustrations of a particular anti-HLA-G/anti-CD3 bispecific antibody (FIG. 1A) and a particular anti-PD1/anti-LAG3 bispecific antibodies (FIG. 1B) as used in the Examples. These molecules are described in more detail in Example 1. In FIG. 1A, an exemplary bispecific anti-HLA-G/anti-CD3 antibody in 2+1 format is shown (referred to as HLA-G TCB). The molecule comprises a single antigen-binding moiety for CD3, two antigen-binding moieties for HLA-G, and an Fc domain. FIG. 1B shows anti-PD1/anti-LAG3 bispecific antibodies in 1+1 CrossMab format, wherein the PD1 binding domain comprises a crossFab (with VH/VL domain exchange) and the LAG3 binding domain comprises CH1 and CK domains with amino acid mutations to support correct pairing (“charged variants”). The Fc part of both antibodies shown here comprises the knob into hole mutations (illustrated by the black circle) and the amino acid mutations L234A, L235A and P329G almost completely abolishing Fcγ receptor binding of the human IgG1 Fc domain.

FIG. 2 shows the protocol of the in vivo efficacy study #1173 of HLA-G TCB (P1AD7977) as monotherapy, and in combination with PD1-LAG3 bispecific antibody (P1AA0927) or with PD-L1 antibody (P1AE0828) versus PD-L1 antibody alone in BC004 PDX (patient derived xenograft)-bearing fully humanized NSG mice. Humanized NSG mice were injected with 2×10⁶ BC004 cells in a total volume of 20 μL PBS into the mammary fat pad (i.m.f.p.). After the tumors reached an average volume of around 200 mm³, mice were randomized to 5 groups receiving: A) histidine buffer (vehicle) as control; B) HLA-G-TCB (0.5 mg/kg once/week intravenously), C) anti PD-L1 antibody (10 mg/kg once/week intravenously), D) HLA-G-TCB (0.5 mg/kg once/week intravenously)+anti PD-L1 antibody (10 mg/kg once/week intravenously), E) HLA-G-TCB (0.5 mg/kg once/week intravenously)+PD1-LAG3 bispecific Ab (3 mg/kg once/week intravenously). In the table underneath the protocol, the subgroups of mice receiving different treatment combinations are defined. The experiment is described in Example 2.

FIGS. 3A and 3B show the results of the study #1173. Tumor volume was measured by caliper 2 times a week. FIG. 3A shows a direct comparison of the results of all subgroups, over a period from day 34 to day 65. Data is shown as tumor volume (Median+/−IQR). In FIG. 3B the measurements of tumor volumes (mm³), over a period from day 34 to day 65, are shown for each individual animal showing homogeneity of anti-tumor response in the various subgroups.

FIG. 4 shows the protocol of the in vivo efficacy study #1655 of HLAG TCB (P1AD7977) as monotherapy, and in combination with PD1-LAG3 bispecific antibody (P1AA0927), at two different concentrations (1.5 mg/kg or 3 mg/kg), or with a combination of PD1 antibody (P1AA6975)+anti-LAG3 antibody (P1AD8676), in BC004 PDX (patient derived xenograft)-bearing fully humanized NSG mice. Humanized NSG mice were injected with 2×10⁶ BC004 cells in a total volume of 20 μL PBS into the mammary fat pad (i.m.f.p.). After the tumors reached an average volume of around 200 mm³, mice were randomized to 5 groups receiving: A) histidine buffer (vehicle) as control; B) HLA-G-TCB (0.5 mg/kg once/week intravenously), C) HLA-G-TCB (0.5 mg/kg once/week intravenously) and anti PD1-LAG3 bispecific antibody (3 mg/kg once/week intravenously), D) HLA-G-TCB (0.5 mg/kg once/week intravenously) and anti PD1-LAG3 bispecific antibody (1.5 mg/kg once/week intravenously), E) HLA-G-TCB (0.5 mg/kg once/week intravenously)+anti-PD1 Ab (1.5 mg/kg once/week intravenously)+anti-LAG3 antibody (1.5 mg/kg once/week intravenously). In the table underneath the protocol, the subgroups of mice receiving different treatment combinations are defined. The experiment is described in Example 2.

FIGS. 5A and 5B show the results of the study #1655. Tumor volume was measured by caliper 2 times a week. FIG. 5A shows a direct comparison of the results of all subgroups, over a period from day 35 to day 60. Data is shown as tumor volume (Median+/−IQR). In FIG. 5B the measurements of tumor volumes (mm³), over a period from day 35 to day 60, are shown for each individual animal showing homogeneity of anti-tumor response in the various subgroups.

FIGS. 6A and 6B show the results of the immunopharmacodynamics analysis performed in study #1655. For this, flow cytometry analysis was performed on treatment Day 17 and after termination on day 32 on single-cell suspensions which were obtained by harvesting and processing tumors and staining them with a cocktail of fluorescently labeled antibodies for detection of immune cell markes. FIG. 6A shows the levels of immune cell infiltration (as indicated by numbers of CD45+ cells) for the different treatment groups on treatment days 17 and 32. FIG. 6B shows the levels of T cell infiltration (as indicated by numbers of CD3+ cells) and of T cell activation (as indicated by Granzyme B expression as percentage of CD8+ T cells) for the different treatment groups on treatment days 17 and 32.

### DETAILED DESCRIPTION OF THE INVENTION

### Definitions

Unless defined otherwise, technical and scientific terms used herein have the same meaning as generally used in the art to which this invention belongs. For purposes of interpreting this specification, the following definitions will apply and whenever appropriate, terms used in the singular will also include the plural and vice versa.

The term “antibody” herein is used in the broadest sense and encompasses various antibody structures, including but not limited to monoclonal antibodies, polyclonal antibodies, monospecific and multispecific antibodies (e.g., bispecific antibodies), and antibody fragments so long as they exhibit the desired antigen-binding activity.

The term “monoclonal antibody” as used herein refers to an antibody obtained from a population of substantially homogeneous antibodies, i.e., the individual antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

The term “monospecific” antibody as used herein denotes an antibody that has one or more binding sites each of which bind to the same epitope of the same antigen. The term “bispecific” means that the antibody is able to specifically bind to at least two distinct antigenic determinants, for example two binding sites each formed by a pair of an antibody heavy chain variable domain (VH) and an antibody light chain variable domain (VL) binding to different antigens or to different epitopes on the same antigen. Such a bispecific antibody is a 1+1 format. Other bispecific antibody formats are 2+1 formats (comprising two binding sites for a first antigen or epitope and one binding site for a second antigen or epitope) or 2+2 formats (comprising two binding sites for a first antigen or epitope and two binding sites for a second antigen or epitope). Typically, a bispecific antibody comprises two antigen binding sites, each of which is specific for a different antigenic determinant.

The term “valent” as used within the current application denotes the presence of a specified number of binding domains in an antigen binding molecule. As such, the terms “bivalent”, “tetravalent”, and “hexavalent” denote the presence of two binding domain, four binding domains, and six binding domains, respectively, in an antigen binding molecule. The bispecific antibodies according to the invention are at least “bivalent” and may be “trivalent” or “multivalent” (e.g. “tetravalent” or “hexavalent”). In a particular aspect, the antibodies of the present invention have two or more binding sites and are bispecific. That is, the antibodies may be bispecific even in cases where there are more than two binding sites (i.e. that the antibody is trivalent or multivalent).

The terms “full length antibody”, “intact antibody”, and “whole antibody” are used herein interchangeably to refer to an antibody having a structure substantially similar to a native antibody structure. “Native antibodies” refer to naturally occurring immunoglobulin molecules with varying structures. For example, native IgG-class antibodies are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a light chain constant domain (CL), also called a light chain constant region. The heavy chain of an antibody may be assigned to one of five types, called α (IgA), δ (IgD), ε (IgE), γ (IgG), or μ (IgM), some of which may be further divided into subtypes, e.g. γ1 (IgG1), γ2 (IgG2), γ3 (IgG3), γ4 (IgG4), α1 (IgA1) and α2 (IgA2). The light chain of an antibody may be assigned to one of two types, called kappa (κ) and lambda (λ), based on the amino acid sequence of its constant domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies, triabodies, tetrabodies, cross-Fab fragments; linear antibodies; single-chain antibody molecules (e.g. scFv); multispecific antibodies formed from antibody fragments and single domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′) 2 fragments comprising salvage receptor binding epitope residues and having increased in vivo half-life, see U.S. Pat. No. 5,869,046. Diabodies are antibody fragments with two antigen binding domains that may be bivalent or bispecific, see, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, MA; see e.g. U.S. Pat. No. 6,248,516 B1). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies. Antibody fragments can be made by various techniques, including but not limited to proteolytic digestion of an intact antibody as well as production by recombinant host cells (e.g. E. coli or phage), as described herein.

Papain digestion of intact antibodies produces two identical antigen-binding fragments, called “Fab” fragments containing each the heavy- and light-chain variable domains and also the constant domain of the light chain and the first constant domain (CH1) of the heavy chain. As used herein, Thus, the term “Fab fragment” refers to an antibody fragment comprising a light chain fragment comprising a VL domain and a constant domain of a light chain (CL), and a VH domain and a first constant domain (CH1) of a heavy chain. Fab′ fragments differ from Fab fragments by the addition of a few residues at the carboxy terminus of the heavy chain CH1 domain including one or more cysteins from the antibody hinge region. Fab′-SH are Fab′ fragments wherein the cysteine residue(s) of the constant domains bear a free thiol group. Pepsin treatment yields an F(ab′)₂ fragment that has two antigen-combining sites (two Fab fragments) and a part of the Fc region.

The term “cross-Fab fragment” or “xFab fragment” or “crossover Fab fragment” refers to a Fab fragment, wherein either the variable regions or the constant regions of the heavy and light chain are exchanged. Two different chain compositions of a crossover Fab molecule are possible and comprised in the bispecific antibodies of the invention: On the one hand, the variable regions of the Fab heavy and light chain are exchanged, i.e. the crossover Fab molecule comprises a peptide chain composed of the light chain variable region (VL) and the heavy chain constant region (CH1), and a peptide chain composed of the heavy chain variable region (VH) and the light chain constant region (CL). This crossover Fab molecule is also referred to as CrossFab_{(VLVII)}. On the other hand, when the constant regions of the Fab heavy and light chain are exchanged, the crossover Fab molecule comprises a peptide chain composed of the heavy chain variable region (VH) and the light chain constant region (CL), and a peptide chain composed of the light chain variable region (VL) and the heavy chain constant region (CH1). This crossover Fab molecule is also referred to as CrossFab_{(CLCH1)}.

A “single chain Fab fragment” or “scFab” is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH—CH1, c) VH-CL-linker-VL-CH1 or d) VL-CH1-linker-VH-CL; and wherein said linker is a polypeptide of at least 30 amino acids, preferably between 32 and 50 amino acids. Said single chain Fab fragments are stabilized via the natural disulfide bond between the CL domain and the CH1 domain. In addition, these single chain Fab molecules might be further stabilized by generation of interchain disulfide bonds via insertion of cysteine residues (e.g. position 44 in the variable heavy chain and position 100 in the variable light chain according to Kabat numbering).

A “crossover single chain Fab fragment” or “x-scFab” is a is a polypeptide consisting of an antibody heavy chain variable domain (VH), an antibody constant domain 1 (CH1), an antibody light chain variable domain (VL), an antibody light chain constant domain (CL) and a linker, wherein said antibody domains and said linker have one of the following orders in N-terminal to C-terminal direction: a) VH-CL-linker-VL-CH1 and b) VL-CH1-linker-VH-CL; wherein VH and VL form together an antigen binding domain which binds specifically to an antigen and wherein said linker is a polypeptide of at least 30 amino acids. In addition, these x-scFab molecules might be further stabilized by generation of interchain disulfide bonds via insertion of cysteine residues (e.g. position 44 in the variable heavy chain and position 100 in the variable light chain according to Kabat numbering).

A “single-chain variable fragment (scFv)” is a fusion protein of the variable regions of the heavy (V_{H}) and light chains (V_{L}) of an antibody, connected with a short linker peptide of ten to about 25 amino acids. The linker is usually rich in glycine for flexibility, as well as serine or threonine for solubility, and can either connect the N-terminus of the V_{H} with the C-terminus of the V_{L}, or vice versa. This protein retains the specificity of the original antibody, despite removal of the constant regions and the introduction of the linker. scFv antibodies are, e.g. described in Houston, J. S., Methods in Enzymol. 203 (1991) 46-96). In addition, antibody fragments comprise single chain polypeptides having the characteristics of a VH domain, namely being able to assemble together with a VL domain, or of a VL domain, namely being able to assemble together with a VH domain to a functional antigen binding site and thereby providing the antigen binding property of full length antibodies.

“Scaffold antigen binding proteins” are known in the art, for example, fibronectin and designed ankyrin repeat proteins (DARPins) have been used as alternative scaffolds for antigen-binding domains, see, e.g., Gebauer and Skerra, Engineered protein scaffolds as next-generation antibody therapeutics. Curr Opin Chem Biol 13:245-255 (2009) and Stumpp et al., Darpins: A new generation of protein therapeutics. Drug Discovery Today 13:695-701 (2008). In one aspect of the invention, a scaffold antigen binding protein is selected from the group consisting of CTLA-4 (Evibody), Lipocalins (Anticalin), a Protein A-derived molecule such as Z-domain of Protein A (Affibody), an A-domain (Avimer/Maxibody), a serum transferrin (trans-body); a designed ankyrin repeat protein (DARPin), a variable domain of antibody light chain or heavy chain (single-domain antibody, sdAb), a variable domain of antibody heavy chain (nanobody, aVH), V_{NAR} fragments, a fibronectin (AdNectin), a C-type lectin domain (Tetranectin); a variable domain of a new antigen receptor beta-lactamase (VNAR fragments), a human gamma-crystallin or ubiquitin (Affilin molecules); a kunitz type domain of human protease inhibitors, microbodies such as the proteins from the knottin family, peptide aptamers and fibronectin (adnectin). CTLA-4 (Cytotoxic T Lymphocyte-associated Antigen 4) is a CD28-family receptor expressed on mainly CD4+ T cells. Its extracellular domain has a variable domain-like Ig fold. Loops corresponding to CDRs of antibodies can be substituted with heterologous sequence to confer different binding properties. CTLA-4 molecules engineered to have different binding specificities are also known as Evibodies (e.g. U.S. Pat. No. 7,166,697B1). Evibodies are around the same size as the isolated variable region of an antibody (e.g. a domain antibody). For further details see Journal of Immunological Methods 248 (1-2), 31-45 (2001). Lipocalins are a family of extracellular proteins which transport small hydrophobic molecules such as steroids, bilins, retinoids and lipids. They have a rigid beta-sheet secondary structure with a number of loops at the open end of the conical structure which can be engineered to bind to different target antigens. Anticalins are between 160-180 amino acids in size, and are derived from lipocalins. For further details see Biochim Biophys Acta 1482:337-350 (2000), U.S. Pat. No. 7,250,297B1 and US20070224633. An affibody is a scaffold derived from Protein A of Staphylococcus aureus which can be engineered to bind to antigen. The domain consists of a three-helical bundle of approximately 58 amino acids. Libraries have been generated by randomization of surface residues. For further details see Protein Eng. Des. Sel. 2004, 17, 455-462 and EP 1641818A1. Avimers are multidomain proteins derived from the A-domain scaffold family. The native domains of approximately 35 amino acids adopt a defined disulfide bonded structure. Diversity is generated by shuffling of the natural variation exhibited by the family of A-domains. For further details see Nature Biotechnology 23 (12), 1556-1561 (2005) and Expert Opinion on Investigational Drugs 16 (6), 909-917 (June 2007). A transferrin is a monomeric serum transport glycoprotein. Transferrins can be engineered to bind different target antigens by insertion of peptide sequences in a permissive surface loop. Examples of engineered transferrin scaffolds include the Trans-body. For further details see J. Biol. Chem 274, 24066-24073 (1999). Designed Ankyrin Repeat Proteins (DARPins) are derived from Ankyrin which is a family of proteins that mediate attachment of integral membrane proteins to the cytoskeleton. A single ankyrin repeat is a 33 residue motif consisting of two alpha-helices and a beta-turn. They can be engineered to bind different target antigens by randomizing residues in the first alpha-helix and a beta-turn of each repeat. Their binding interface can be increased by increasing the number of modules (a method of affinity maturation). For further details see J. Mol. Biol. 332, 489-503 (2003), PNAS 100 (4), 1700-1705 (2003) and J. Mol. Biol. 369, 1015-1028 (2007) and US20040132028A1.

A single-domain antibody is an antibody fragment consisting of a single monomeric variable antibody domain. The first single domains were derived from the variable domain of the antibody heavy chain from camelids (nanobodies or V_{H}H fragments). Furthermore, the term single-domain antibody includes an autonomous human heavy chain variable domain (aVH) or V_{NAR} fragments derived from sharks. Fibronectin is a scaffold which can be engineered to bind to antigen. Adnectins consists of a backbone of the natural amino acid sequence of the 10th domain of the 15 repeating units of human fibronectin type III (FN3). Three loops at one end of the .beta.-sandwich can be engineered to enable an Adnectin to specifically recognize a therapeutic target of interest. For further details see Protein Eng. Des. Sel. 18, 435-444 (2005), US20080139791. WO2005056764 and U.S. Pat. No. 6,818,418B1. Peptide aptamers are combinatorial recognition molecules that consist of a constant scaffold protein, typically thioredoxin (TrxA) which contains a constrained variable peptide loop inserted at the active site. For further details see Expert Opin. Biol. Ther. 5, 783-797 (2005). Microbodies are derived from naturally occurring microproteins of 25-50 amino acids in length which contain 3-4 cysteine bridges—examples of microproteins include KalataBI and conotoxin and knottins. The microproteins have a loop which can beengineered to include upto 25 amino acids without affecting the overall fold of the microprotein. For further details of engineered knottin domains, see WO2008098796.

An “antigen binding molecule that binds to the same epitope” as a reference molecule refers to an antigen binding molecule that blocks binding of the reference molecule to its antigen in a competition assay by 50% or more, and conversely, the reference molecule blocks binding of the antigen binding molecule to its antigen in a competition assay by 50% or more.

As used herein, the term “antigen binding domain” or “antigen-binding site” refers to the part of the antigen binding molecule that specifically binds to an antigenic determinant. More particularly, the term “antigen-binding domain” refers the part of an antibody that comprises the area which specifically binds to and is complementary to part or all of an antigen. Where an antigen is large, an antigen binding molecule may only bind to a particular part of the antigen, which part is termed an epitope. An antigen binding domain may be provided by, for example, one or more variable domains (also called variable regions). Preferably, an antigen binding domain comprises an antibody light chain variable region (VL) and an antibody heavy chain variable region (VH). In one aspect, the antigen binding domain is able to bind to its antigen and block or partly block its function. Antigen binding domains that specifically bind to PD1 or to LAG3 include antibodies and fragments thereof as further defined herein. In addition, antigen binding domains may include scaffold antigen binding proteins, e.g. binding domains which are based on designed repeat proteins or designed repeat domains (see e.g. WO 2002/020565).

As used herein, the term “antigenic determinant” is synonymous with “antigen” and “epitope.” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins useful as antigens herein can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁷ M or less, e.g. from 10⁻⁷ M to 10⁻¹³ M, e.g. from 10⁻⁹ M to 10⁻¹³ M).

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (K_{D})), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

As used herein, the term “high affinity” of an antibody refers to an antibody having a K_{D} of 10⁻⁹ M or less and even more particularly 10⁻¹⁰ M or less for a target antigen. The term “low affinity” of an antibody refers to an antibody having a K_{D} of 10⁻⁸ or higher.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

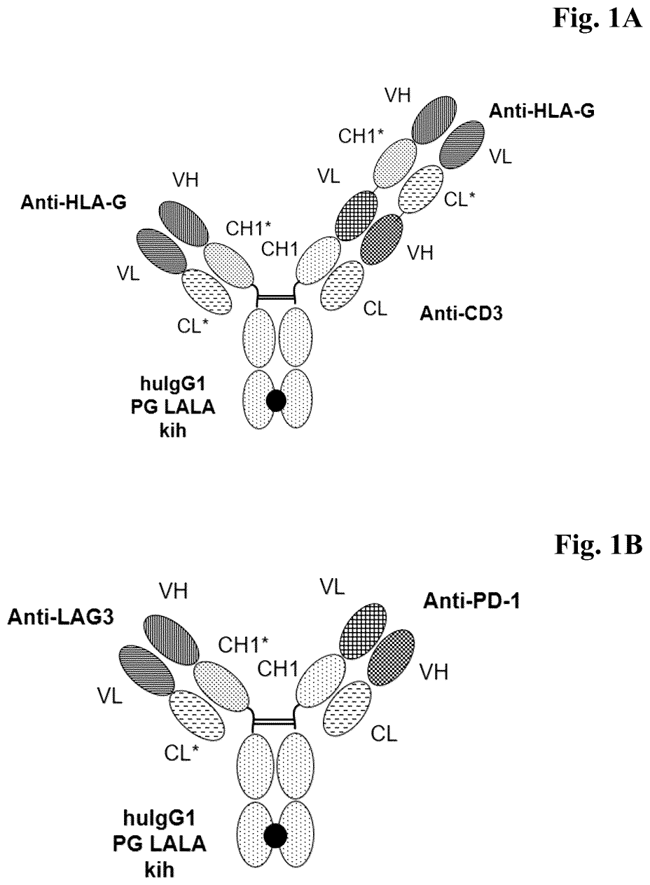

When used herein, the term “HLA-G”, “human HLA-G”, “HLAG”, refers to the HLA-G human major histocompatibility complex, class I, G, also known as human leukocyte antigen G (HLA-G) (exemplary SEQ ID NO:61). Typically, HLA-G forms a MHC class I complex together with β2 microglobulin (B2M or β2m). In one embodiment, HLA-G refers to the MHC class I complex of HLA-G and β2 microglobulin. In one preferred embodiment, HLA-G refers to the cell surface bound MHC class I complex of HLA-G and β2 microglobulin, also known as HLA-G1 (see e.g. WO2022/129120 A1, FIG. 1, and Blaschitz et al., Molecular Human Reproduction, 11 (2005) 699-710, inter alia FIG. 1).

As used herein, an antibody (either mono-, multi- or bispecific) or antigen-binding domain “binding to human HLA-G”, “specifically binding to (human) HLA-G”, “that binds to (human) HLA-G” or “anti-HLA-G” refers to an antibody/antigen-binding moiety specifically binding to the (human) HLA-G antigen or its extracellular domain (ECD) with a binding affinity of a K_{D}-value of 5.0×10⁻⁸ mol/l or lower, in one embodiment of a K_{D}-value of 1.0×10⁻⁹ mol/l or lower, in one embodiment of a K_{D}-value of 5.0×10⁻⁸ mol/l to 1.0×10⁻¹³ mol/l. In one embodiment the antibody binds to HLA-G B2M MHC I complex comprising SEQ ID NO:106).

The binding affinity is determined with a standard binding assay, such as surface plasmon resonance technique (BIAcore®, GE-Healthcare Uppsala, Sweden) e.g. using constructs comprising HLA-G extracellular domain (e.g. in its natural occurring three-dimensional structure). In one embodiment, binding affinity is determined with a standard binding assay using exemplary soluble HLA-G comprising MHC class I complex comprising SEQ ID NO:106.

HLA-G has the regular MHC I fold and consists of two chains: Chain 1 consists of three domains: alpha 1, alpha 2 and alpha 3. The alpha 1 and alpha 2 domains form a peptide binding groove flanked by two alpha helices. Small peptides (approximately 9mers) can bind to this groove akin to other MHCI proteins. Chain 2 is beta 2 microglobulin (β2M) which is shared with various other MHCI proteins.

HLA-G can form functionally active complex oligomeric structures (Kuroki, K et al. Eur J Immunol. 37 (2007) 1727-1729). Disulfide-linked dimers are formed between Cys 42 of two HLA-G molecules. (Shiroishi M et al., J Biol Chem 281 (2006) 10439-10447. Trimers and Tetrameric complexes have also been described e.g. in Kuroki, K et al. Eur J Immunol. 37 (2007) 1727-1729, Allan D. S., et al. J Immunol Methods. 268 (2002) 43-50 and T Gonen-Gross et al., J Immunol 171 (2003) 1343-1351). HLA-G has several free cysteine residues, unlike most of the other MHC class I molecules. Boyson et al., Proc Nat Acad Sci USA, 99:16180 (2002) reported that the recombinant soluble form of HLA-G5 could form a disulfide-linked dimer with the intermolecular Cys42-Cys42 disulfide bond. In addition, the membrane-bound form of HLA-G1 can also form a disulfide-linked dimer on the cell surface of the JEG3 cell line, which endogenously expresses HLA-G. Disulfide-linked dimer forms of HLA-G1 and HLA-G5 have been found on the cell surface of trophoblast cells as well (Apps, R., Tissue Antigens, 68:359 (2006)).

HLA-G is predominantly expressed on cytotrophoblasts in the placenta. Several tumors (including pancreatic, breast, skin, colorectal, gastric & ovarian) express HLA-G (Lin, A. et al., Mol Med. 21 (2015) 782-791; Amiot, L., et al., Cell Mol Life Sci. 68 (2011) 417-431). The expression has also been reported to be associated with pathological conditions like inflammatory diseases, GvHD and cancer. Expression of HLA-G has been reported to be associated with poor prognosis in cancer. Tumor cells escape host immune surveillance by inducing immune tolerance/suppression via HLA-G expression.

For HLA-G, 7 isoforms exist, whereof 3 are secreted and 4 are membrane bound forms. The most important functional isoforms of HLA-G include beta-2 microglobulin (β2M)-associated HLA-G1 and HLA-G5. However, the tolerogenic immunological effect of these isoforms is different and is dependent on the form (monomer, dimer) of ligands and the affinity of the ligand-receptor interaction.

HLA-G protein can be produced using standard molecular biology techniques. The nucleic acid sequence for HLA-G isoforms is known in the art. See for example GenBank Accession No. AY359818.

The HLA-G isomeric forms promote signal transduction through ILTs (Ig-like transcripts), in particular ILT2, ILT4, or a combination thereof.

ILTs represent Ig types of activating and inhibitory receptors that are involved in regulation of immune cell activation and control the function of immune cells (Borges, L., et al., Curr Top Microbial Immunol, 244:123-136 (1999)). ILTs are categorized into three groups: (i) inhibitory, i.e. those containing a cytoplasmic immunoreceptor tyrosine-based inhibitory motif (ITIM) and transducing an inhibitory signal (ILT2, ILT3, ILT4, ILT5, and LIR8); (ii) activating, i.e. those containing a short cytoplasmic tail and a charged amino acid residue in the transmembrane domain (ILT1, ILT7, ILT8, and LIR6alpha) and delivering an activating signal through the cytoplasmic immunoreceptor tyrosine-based activating motif (ITAM) of the associated common gamma chain of Fc receptor; and (iii) the soluble molecule ILT6 which lacks the transmembrane domain. A number of recent studies have highlighted immunoregulatory roles for ILTs on the surface of antigen presenting cells (APC). ILT2, ILT3, and ILT4 receptors, the most characterized immune inhibitory receptors, are expressed on a wide range of immune cells including monocytes, B cells, dendritic cells, plasmacytoid dendritic cells and a subset of NK and T cells. ILT2 is expressed on T cells subsets and has been shown to inhibit activation and proliferation of these cells upon ligation (Colonna M. et al., J Immunol. 20011, 66:2514-2521, J Immunol 2000; 165:3742-3755). ILT3 and ILT4 are upregulated by exposing immature DC to known immunosuppressive factors, including IL-10, vitamin D3, or suppressor CD8 T cells (Chang, C. C., et al., Nat Immunol, 3:237-243 (2002)). The expression of ILTs on DC is tightly controlled by inflammatory stimuli, cytokines, and growth factors, and is down-regulated following DC activation (Ju, X. S., et al., Gene, 331:159-164 (2004)). The expression of ILT2 and ILT4 receptors is highly regulated by histone acetylation, which contributes to strictly controlled gene expression exclusively in the myeloid lineage of cells (Nakajima, H., J Immunol, 171:6611-6620 (2003)).

Engagement of the inhibitory receptors ILT2 and ILT4 alters the cytokine and chemokine secretion/release profile of monocytes and can inhibit Fc receptor signaling (Colonna, M., et al. J Leukoc Biol, 66:375-381 (1999)). The role and function of ILT3 on DC have been precisely described by the Suciu-Foca group (Suciu-Foca, N., Int Immunopharmacol, 5:7-11 (2005)). Although the ligand for ILT3 is unknown, ILT4 is known to bind to the third domain of HLA class I molecules (HLA-A, HLA-B, HLA-C, and HLA-G), competing with CD8 for MHC class I binding (Shiroishi, M., Proc Natl Acad Sci USA, 100:8856-8861 (2003)). The preferential ligand for several inhibitory ILT receptors is HLA-G. HLA-G plays a potential role in maternal-fetal tolerance and in the mechanisms of escape of tumor cells from immune recognition and destruction (Hunt, J. S., et al., Faseb J. 19:681-693 (2005)). It is most likely that regulation of DC function by HLA-G-ILT interactions is an important pathway in the biology of DC. It has been determined that human monocyte-derived DC that highly express ILT2 and ILT4 receptors, when treated with HLA-G and stimulated with allogeneic T cells, still maintain a stable tolerogenic-like phenotype (CD80low, CD86low, HLA-DRlow) with the potential to induce T cell anergy (Ristich, V., et al., Eur J Immunol, 35:1133-1142 (2005)). Moreover, the HLA-G interaction with DC that highly express ILT2 and ILT4 receptors resulted in down-regulation of several genes involved in the MHC class II presentation pathway. A lysosomal thiol reductase, IFN-gamma inducible lysosomal thiol reductase (GILT), abundantly expressed by professional APC, was greatly reduced in HLA-G-modified DC. The repertoire of primed CD4+ T cells can be influenced by DC expression of GILT, as in vivo T cell responses to select antigens were reduced in animals lacking GILT after targeted gene disruption (Marie, M., et al., Science, 294:1361-1365 (2001)). The HLA-G/ILT interaction on DC interferes with the assembly and transport of MHC class II molecules to the cell surface, which might result in less efficient presentation or expression of structurally abnormal MHC class II molecules. It was determined that HLA-G markedly decreased the transcription of invariant chain (CD74), HLA-DMA, and HLA-DMB genes on human monocyte-derived DC highly expressing ILT inhibitory receptors (Ristich, V., et al; Eur J Immunol 35:1133-1142 (2005)).

Another receptor of HLA-G is KIR2DL4 because KIR2DL4 binds to cells expressing HLA-G (US2003232051; Cantoni, C. et al. Eur J Immunol 28 (1998) 1980; Rajagopalan, S. and E. O. Long. [published erratum appears in J Exp Med 191 (2000) 2027] J Exp Med 189 (1999) 1093; Ponte, M. et al. PNAS USA 96 (1999) 5674). KIR2DL4 (also referred to as 2DL4) is a KIR family member (also designated CD158d) that shares structural features with both activating and inhibitory receptors (Selvakumar, A. et al. Tissue Antigens 48 (1996) 285). 2DL4 has a cytoplasmic ITIM, suggesting inhibitory function, and a positively charged amino acid in the transmembrane region, a feature typical of activating KIR. Unlike other clonally distributed KIRs, 2DL4 is transcribed by all NK cells (Valiante, N. M. et al. Immunity 7 (1997) 739; Cantoni, C. et al. Eur J Immunol 28 (1998) 1980; Rajagopalan, S. and E. O. Long. [published erratum appears in J Exp Med 191 (2000) 2027] J Exp Med 189 (1999) 1093).

The term “inhibits ILT2 binding to HLA-G on JEG-3 cells (ATCC HTB36)” refers to the inhibition of binding interaction of (recombinant) ILT2 e.g in an assay as described in Example 5 of WO2022/129120.

The term “reduction” (and grammatical variations thereof such as “reduce” or “reducing”), for example reduction of the number of B cells or of cytokine release, refers to a decrease in the respective quantity, as measured by appropriate methods known in the art. For clarity the term includes also reduction to zero (or below the detection limit of the analytical method), i.e. complete abolishment or elimination. Conversely, “increased” refers to an increase in the respective quantity.

A “T cell antigen” as used herein refers to an antigenic determinant presented on the surface of a T lymphocyte, particularly a cytotoxic T lymphocyte.

A “T cell activating therapeutic agent” as used herein refers to a therapeutic agent capable of inducing T cell activation in a subject, particularly a therapeutic agent designed for inducing T cell activation in a subject. Examples of T cell activating therapeutic agents include bispecific antibodies that specifically bind an activating T cell antigen (also referred to as “T cell bispecific antibodies” or “TCBs”), such as CD3, and a target cell antigen, such as HLA-G. Further examples include chimeric antigen receptors (CARs) which comprise a T cell activating domain and an antigen binding moiety that specifically binds to a target cell antigen, such as HLA-G.

An “activating T cell antigen” as used herein refers to an antigenic determinant expressed by a T lymphocyte, particularly a cytotoxic T lymphocyte, which is capable of inducing or enhancing T cell activation upon interaction with an antigen binding molecule. Specifically, interaction of an antigen binding molecule with an activating T cell antigen may induce T cell activation by triggering the signaling cascade of the T cell receptor complex. An exemplary activating T cell antigen is CD3.

The term “CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length.” unprocessed CD3 as well as any form of CD3 that results from processing in the cell. The term also encompasses naturally occurring variants of CD3. e.g., splice variants or allelic variants. In one embodiment. CD3 is human CD3, particularly the epsilon subunit of human CD3 (CD38). The amino acid sequence of human CD38 is shown in UniProt (www.uniprot.org) accession no. P07766 (version 144), or NCBI (www.ncbi.nlm.nih.gov/) RefSeq NP_000724.1. See also SEQ ID NO:66. The amino acid sequence of cynomolgus [Macaca fascicularis] CD38 is shown in NCBI GenBank no. BAB71849.1. See also SEQ ID NO:67.

The terms “a bispecific antibody comprising a first antigen binding domain that specifically binds to CD3 and a second antigen binding domain that specifically binds to HLA-G” “a bispecific antibody that specifically binds CD3 and HLA-G”. “bispecific antigen binding molecule specific for CD3 and HLA-G”, an “anti-HLA-G/anti-CD3 bispecific antibody”, an “HLA-G CD3 TCB” or an “HLA-G TCB” are used interchangeably herein and refer to a bispecific antibody that is capable of binding CD3 and HLA-G with sufficient affinity such that the antibody is useful as a diagnostic and/or therapeutic agent in targeting CD3 and HLA-G.

The term “PD1”, also known as Programmed cell death protein 1, is a type I membrane protein of 288 amino acids that was first described in 1992 (Ishida et al., EMBO J., 11 (1992). 3887-3895). PD1 is a member of the extended CD28/CTLA-4 family of T cell regulators and has two ligands. PD-L1 (B7-H1. CD274) and PD-L2 (B7-DC. CD273). The protein's structure includes an extracellular IgV domain followed by a transmembrane region and an intracellular tail. The intracellular tail contains two phosphorylation sites located in an immunoreceptor tyrosine-based inhibitory motif and an immunoreceptor tyrosine-based switch motif, which suggests that PD1 negatively regulates TCR signals. This is consistent with binding of SHP-1 and SHP-2 phosphatases to the cytoplasmic tail of PD-1 upon ligand binding. While PD1 is not expressed on naïve T cells, it is upregulated following T cell receptor (TCR)-mediated activation and is observed on both activated and exhausted T cells (Agata et al., Int. Immunology 8 (1996). 765-772). These exhausted T cells have a dysfunctional phenotype and are unable to respond appropriately. Although PD1 has a relatively wide expression pattern, its most important role is likely as a coinhibitory receptor on T cells (Chinai et al. Trends in Pharmacological Sciences 36 (2015). 587-595). Current therapeutic approaches thus focus on blocking the interaction of PD1 with its ligands to enhance T cell response. The terms “Programmed Death 1.” “Programmed Cell Death 1.” “Protein PD-1,” “PD-1.” PD1.” “PDCD1,” “hPD-1” and “hPD-I” can be used interchangeably, and include variants, isoforms, species homologs of human PD1, and analogs having at least one common epitope with PD-1. The amino acid sequence of human PD1 is shown in UniProt (www.uniprot.org) accession no. Q15116 (SEQ ID NO:68).

The terms “anti-PD1 antibody” and “an antibody comprising an antigen binding domain that binds to PD1” refer to an antibody that is capable of binding PD1, especially a PD1 polypeptide expressed on a cell surface, with sufficient affinity such that the antibody is useful as a diagnostic and/or therapeutic agent in targeting PD1. In one aspect, the extent of binding of an anti-PD1 antibody to an unrelated, non-PD1 protein is less than about 10% of the binding of the antibody to PD1 as measured, e.g., by radioimmunoassay (RIA) or flow cytometry (FACS) or by a Surface Plasmon Resonance assay using a biosensor system such as a Biacore® system. In certain aspects, an antigen binding protein that binds to human PD1 has a K_{D} value of the binding affinity for binding to human PD1 of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M). In one preferred embodiment the respective K_{D} value of the binding affinities is determined in a Surface Plasmon Resonance assay using the Extracellular domain (ECD) of human PD1 (PD1-ECD) for the PD1 binding affinity. The term “anti-PD1 antibody” also encompasses bispecific antibodies that are capable of binding PD1 and a second antigen.

In a specific aspect, the anti-PD1 antibody is selected from the group consisting of MDX 1106 (nivolumab), MK-3475 (pembrolizumab), CT-011 (pidilizumab), PDR001 (spartalizumab), SHR 1210 (camrelizumab), MEDI-0680 (AMP-514), REGN2810, and BGB-108. In one particular aspect, the anti-PD1 antibody is pembrolizumab, or an antibody comprising a heavy chain comprising the amino acid sequence of SEQ ID NO:75 and a light chain comprising the amino acid sequence of SEQ ID NO:76. Pembrolizumab (Merck), also known as MK-3475, Merck 3475, lambrolizumab, SCH-900475, and KEYTRUDA®, is an anti-PD1 antibody described in WO 2009/114335 (CAS Reg. No. 1374853-91-4). In one particular aspect, the anti-PD1 antibody is nivolumab, or an antibody comprising a heavy chain comprising the amino acid sequence of SEQ ID NO: 77 and a light chain comprising the amino acid sequence of SEQ ID NO:78. Nivolumab (CAS Registry Number: 946414-94-4, Bristol-Myers Squibb/Ono), also known as MDX-1106-04, MDX-1106, ONO-4538, BMS-936558, and OPDIVO®, is an anti-PD1 antibody described in WO 2006/121168 (CAS Reg. No. 946414-94-4). In another particular aspect, the anti-PD1 antibody comprises a heavy chain variable domain VH comprising the amino acid sequence of SEQ ID NO: 7 and a light chain variable domain VL comprising the amino acid sequence of SEQ ID NO:8, or a humanized variant thereof. In a particular aspect, the anti-PD1 antibody comprises a heavy chain variable domain VH comprising the amino acid sequence of SEQ ID NO:9 and a light chain variable domain VL comprising the amino acid sequence of SEQ ID NO:10.

The terms “LAG3” or “Lag-3” or “Lymphocyte activation gene-3” or “CD223” as used herein refer to any native LAG3 from any vertebrate source, including mammals such as primates (e.g. humans) and rodents (e.g., mice and rats), unless otherwise indicated. The term encompasses “full-length.” unprocessed LAG3 as well as any form of LAG3 resulting from processing in the cell. The term also encompasses naturally occurring variants of LAG3, e.g., splice variants or allelic variants. In one preferred embodiment the term “LAG3” refers to human LAG3. The amino acid sequence of an exemplary processed (without signal sequences) LAG3 is shown in SEQ ID NO: 69. The amino acid sequence of an exemplary Extracellular Domain (ECD) LAG3 is shown in SEQ ID NO:70.

The terms “anti-LAG3 antibody” and “an antibody that binds to LAG3” refer to an antibody that is capable of binding LAG3 with sufficient affinity such that the antibody is useful as a diagnostic and/or therapeutic agent in targeting LAG3. In one aspect, the extent of binding of an anti-LAG3 antibody to an unrelated, non-LAG3 protein is less than about 10% of the binding of the antibody to LAG3 as measured, e.g., by a radioimmunoassay (RIA). In certain embodiments, an antibody that binds to LAG3 has a dissociation constant (K_{D})) of ≤1 μM, ≤100 nM, ≤10 nM, ≤1 nM, ≤0.1 nM, ≤0.01 nM, or ≤0.001 nM (e.g. 108 M or less, e.g. from 10⁻⁸ M to 10⁻¹³ M. e.g., from 10⁻⁹ M to 10⁻¹³ M). In certain aspects, an anti-LAG3 antibody binds to an epitope of LAG3 that is conserved among LAG3 from different species. In one preferred embodiment, an “anti-LAG3 antibody”. “an antibody that specifically binds to human LAG3”, and “an antibody that binds to human LAG3” refers to an antibody specifically binding to the human LAG3 antigen or its Extracellular Domain (ECD) with a binding affinity of a K_{D}-value of 1.0×10⁻⁸ mol/l or lower, in one embodiment of a K_{D}-value of 1.0×10⁻⁹ mol/l or lower, in one embodiment of a K_{D}-value of 1.0×10⁻⁹ mol/l to 1.0×10⁻¹³ mol/l. In this context the binding affinity is determined with a standard binding assay, such as surface plasmon resonance technique (BIAcore®, GE-Healthcare Uppsala. Sweden) e.g. using the LAG3 extracellular domain. The term “anti-LAG3 antibody” also encompasses bispecific antibodies that are capable of binding LAG3 and a second antigen. In one aspect, the anti-LAG3 antibody is relatlimab or BMS-986016, or an antibody comprising a heavy chain variable domain comprising the amino acid sequence of SEQ ID NO:27 and a light chain variable domain comprising the amino acid sequence of SEQ ID NO:28. In a particular aspect, the anti-LAG3 antibody comprises a heavy chain variable domain VH comprising the amino acid sequence of SEQ ID NO:17 and a light chain variable domain VL comprising the amino acid sequence of SEQ ID NO:18, or a heavy chain variable domain VH comprising the amino acid sequence of SEQ ID NO:25 and a light chain variable domain VL comprising the amino acid sequence of SEQ ID NO:26, or a heavy chain variable domain VH comprising the amino acid sequence of SEQ ID NO:64 and a light chain variable domain VL comprising the amino acid sequence of SEQ ID NO:65.

The terms “a bispecific antibody comprising a first antigen binding domain that specifically binds to PD1 and a second antigen binding domain that specifically binds to LAG3” “a bispecific antibody that specifically binds PD1 and LAG3”, “bispecific antigen binding molecule specific for PD1 and LAG3” or an “anti-PD1/anti-LAG3 (bispecific) antibody” are used interchangeably herein and refer to a bispecific antibody that is capable of binding PD1 and LAG3 with sufficient affinity such that the antibody is useful as a diagnostic and/or therapeutic agent in targeting PD1 and LAG3.

A “blocking” antibody or an “antagonist” antibody is one that inhibits or reduces a biological activity of the antigen it binds. In some embodiments, blocking antibodies or antagonist antibodies substantially or completely inhibit the biological activity of the antigen. For example, the bispecific antibodies of the invention block the signaling through PD1 and LAG3 so as to restore a functional response by T cells (e.g., proliferation, cytokine production, target cell killing) from a dysfunctional state to antigen stimulation.

The term “variable region” or “variable domain” refers to the domain of an antibody heavy or light chain that is involved in binding the antigen binding molecule to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6th ed., W.H. Freeman and Co., page 91 (2007). A single VH or VL domain may be sufficient to confer antigen-binding specificity.

The term “hypervariable region” or “HVR” as used herein refers to each of the regions of an antibody variable domain which are hypervariable in sequence and which determine antigen binding specificity, for example “complementarity determining regions” (“CDRs”). Generally, antibodies comprise six CDRs: three in the VH (CDR-H1, CDR-H2, CDR-H3), and three in the VL (CDR-L1, CDR-L2, CDR-L3). Exemplary CDRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991)); and
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262:732-745 (1996)).

Unless otherwise indicated, the CDRs are determined according to Kabat et al., supra. One of skill in the art will understand that the CDR designations can also be determined according to Chothia, supra, McCallum, supra, or any other scientifically accepted nomenclature system.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc., according to Kabat) after heavy chain FR residue 82. The Kabat numbering of residues may be determined for a given antibody by alignment at regions of homology of the sequence of the antibody with a “standard” Kabat numbered sequence. Generally, native four-chain antibodies comprise six HVRs; three in the VH (H1, H2, H3), and three in the VL (L1, L2, L3).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1 (L1)-FR2-H2 (L2)-FR3-H3 (L3)-FR4.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g. IgG₁, IgG₂, IgG₃, IgG₄, IgA₁, and IgA₂. The heavy chain constant domains that correspond to the different classes of immunoglobulins are called α, δ, ε, γ, and μ respectively.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization. Other forms of “humanized antibodies” encompassed by the present invention are those in which the constant region has been additionally modified or changed from that of the original antibody to generate the properties according to the invention, especially in regard to Clq binding and/or Fc receptor (FcR) binding.

A “human” antibody is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “Fc domain” or “Fc region” herein is used to define a C-terminal region of an antibody heavy chain that contains at least a portion of the constant region. The term includes native sequence Fc regions and variant Fc regions. Particularly, a human IgG heavy chain Fc region extends from Cys226, or from Pro230, to the carboxyl-terminus of the heavy chain. However, the C-terminal lysine (Lys447) of the Fc region may or may not be present. The amino acid sequences of the heavy chains are always presented with the C-terminal lysine, however variants without the C-terminal lysine are included in the invention.

An IgG Fc region comprises an IgG CH2 and an IgG CH3 domain. The “CH2 domain” of a human IgG Fc region usually extends from an amino acid residue at about position 231 to an amino acid residue at about position 340. In one embodiment, a carbohydrate chain is attached to the CH2 domain. The CH2 domain herein may be a native sequence CH2 domain or variant CH2 domain. The “CH3 domain” comprises the stretch of residues C-terminal to a CH2 domain in an Fc region (i.e. from an amino acid residue at about position 341 to an amino acid residue at about position 447 of an IgG). The CH3 region herein may be a native sequence CH3 domain or a variant CH3 domain (e.g. a CH3 domain with an introduced “protuberance” (“knob”) in one chain thereof and a corresponding introduced “cavity” (“hole”) in the other chain thereof; see U.S. Pat. No. 5,821,333, expressly incorporated herein by reference). Such variant CH3 domains may be used to promote heterodimerization of two non-identical antibody heavy chains as herein described. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

The “knob-into-hole” technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine). The protuberance and cavity can be made by altering the nucleic acid encoding the polypeptides, e.g. by site-specific mutagenesis, or by peptide synthesis. In a specific embodiment a knob modification comprises the amino acid substitution T366W in one of the two subunits of the Fc domain, and the hole modification comprises the amino acid substitutions T366S, L368A and Y407V in the other one of the two subunits of the Fc domain. In a further specific embodiment, the subunit of the Fc domain comprising the knob modification additionally comprises the amino acid substitution S354C, and the subunit of the Fc domain comprising the hole modification additionally comprises the amino acid substitution Y349C. Introduction of these two cysteine residues results in the formation of a disulfide bridge between the two subunits of the Fc region, thus further stabilizing the dimer (Carter, J Immunol Methods 248, 7-15 (2001)).

A “region equivalent to the Fc region of an immunoglobulin” is intended to include naturally occurring allelic variants of the Fc region of an immunoglobulin as well as variants having alterations which produce substitutions, additions, or deletions but which do not decrease substantially the ability of the immunoglobulin to mediate effector functions (such as antibody-dependent cellular cytotoxicity). For example, one or more amino acids can be deleted from the N-terminus or C-terminus of the Fc region of an immunoglobulin without substantial loss of biological function. Such variants can be selected according to general rules known in the art so as to have minimal effect on activity (see, e.g., Bowie, J. U. et al., Science 247:1306-10 (1990)).

The term “effector functions” refers to those biological activities attributable to the Fc region of an antibody, which vary with the antibody isotype. Examples of antibody effector functions include: Clq binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

An “activating Fc receptor” is an Fc receptor that following engagement by an Fc region of an antibody elicits signaling events that stimulate the receptor-bearing cell to perform effector functions. Activating Fc receptors include FcγRIIIa (CD16a), FcγRI (CD64), FcγRIIa (CD32), and FcαRI (CD89). A particular activating Fc receptor is human FcγRIIIa (see UniProt accession no. P08637, version 141).

The term “peptide linker” refers to a peptide comprising one or more amino acids, typically about 2 to 20 amino acids. Peptide linkers are known in the art or are described herein. Suitable, non-immunogenic linker peptides are, for example, (G₄S)ₙ, (SG₄)ₙ or G₄(SG₄)ₙ peptide linkers, wherein “n” is generally a number between 1 and 10, typically between 2 and 4, in particular 2. Peptide linkers of particular interest are (G₄S) (SEQ ID NO:71), (G₄S) 2 or GGGGSGGGGS (SEQ ID NO: 72), (G₄S)₃ (SEQ ID NO:73) and (G₄S)₄ (SEQ ID NO:74), more particularly (G₄S)₂ or GGGGSGGGGS (SEQ ID NO:72).

By “fused to” or “connected to” is meant that the components (e.g. an antigen binding domain and a FC domain) are linked by peptide bonds, either directly or via one or more peptide linkers.

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

“Percent (%) amino acid sequence identity” with respect to a reference polypeptide (protein) sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN. SAWI or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

In certain aspects, amino acid sequence variants of the bispecific antibodies of the invention provided herein are contemplated. For example, it may be desirable to improve the binding affinity and/or other biological properties of the bispecific antibodies. Amino acid sequence variants of the bispecific antibodies may be prepared by introducing appropriate modifications into the nucleotide sequence encoding the molecules, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. Sites of interest for substitutional mutagenesis include the HVRs and Framework (FRs). Conservative substitutions are provided in Table A under the heading “Preferred Substitutions” and further described below in reference to amino acid side chain classes (1) to (6). Amino acid substitutions may be introduced into the molecule of interest and the products screened for a desired activity, e.g., retained/improved antigen binding, decreased immunogenicity, or improved ADCC or CDC.

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

The term “amino acid sequence variants” includes substantial variants wherein there are amino acid substitutions in one or more hypervariable region residues of a parent antigen binding molecule (e.g. a humanized or human antibody). Generally, the resulting variant(s) selected for further study will have modifications (e.g., improvements) in certain biological properties (e.g., increased affinity, reduced immunogenicity) relative to the parent antigen binding molecule and/or will have substantially retained certain biological properties of the parent antigen binding molecule. An exemplary substitutional variant is an affinity matured antibody, which may be conveniently generated, e.g., using phage display-based affinity maturation techniques such as those described herein. Briefly, one or more HVR residues are mutated and the variant antigen binding molecules displayed on phage and screened for a particular biological activity (e.g. binding affinity). In certain embodiments, substitutions, insertions, or deletions may occur within one or more HVRs so long as such alterations do not substantially reduce the ability of the antigen binding molecule to bind antigen. For example, conservative alterations (e.g., conservative substitutions as provided herein) that do not substantially reduce binding affinity may be made in HVRs. A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp. His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antigen binding molecule complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include bispecific antibodies with an N-terminal methionyl residue. Other insertional variants of the molecule include the fusion to the N- or C-terminus to a polypeptide which increases the serum half-life of the bispecific antibody.

In certain aspects, the bispecific antibodies provided herein are altered to increase or decrease the extent to which the antibody is glycosylated. Glycosylation variants of the molecules may be conveniently obtained by altering the amino acid sequence such that one or more glycosylation sites is created or removed, e.g. the carbohydrates attached to the Fc domain may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in the bispecific antibodies of the invention may be made in order to create variants with certain improved properties. In one aspect, variants of bispecific antibodies are provided having a carbohydrate structure that lacks fucose attached (directly or indirectly) to an Fc region. Such fucosylation variants may have improved ADCC function, see e.g. US Patent Publication Nos. US 2003/0157108 (Presta, L.) or US 2004/0093621 (Kyowa Hakko Kogyo Co., Ltd). Further variants of the bispecific antibodies of the invention include those with bisected oligosaccharides, e.g., in which a biantennary oligosaccharide attached to the Fc region is bisected by GlcNAc. Such variants may have reduced fucosylation and/or improved ADCC function., see for example WO 2003/011878 (Jean-Mairet et al.); U.S. Pat. No. 6,602,684 (Umana et al.); and US 2005/0123546 (Umana et al.). Variants with at least one galactose residue in the oligosaccharide attached to the Fc region are also provided. Such antibody variants may have improved CDC function and are described, e.g., in WO 1997/30087 (Patel et al.); WO 1998/58964 (Raju, S.); and WO 1999/22764 (Raju, S.).

In certain aspects, it may be desirable to create cysteine engineered variants of the bispecific antibodies of the invention, e.g., “thioMAbs,” in which one or more residues of the molecule are substituted with cysteine residues. In particular embodiments, the substituted residues occur at accessible sites of the molecule. By substituting those residues with cysteine, reactive thiol groups are thereby positioned at accessible sites of the antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antigen binding molecules may be generated as described, e.g., in U.S. Pat. No. 7,521,541.

In certain aspects, the bispecific antibodies provided herein may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the bispecific antibody derivative will be used in a therapy under defined conditions, etc.

In another aspect, conjugates of an antibody and non-proteinaceous moiety that may be selectively heated by exposure to radiation are provided. In one embodiment, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

An “immunoconjugate” is an antibody conjugated to one or more heterologous molecule(s), including but not limited to a cytotoxic agent.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms.

Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line.” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. In particular, the host cell is a prokaryotic or eukaryotic host cell. Host cells include cultured cells, e.g. mammalian cultured cells, such as CHO cells, BHK cells, NS0 cells, SP2/0 cells, YO myeloma cells, P3×63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable excipient” refers to an ingredient in a pharmaceutical composition, other than an active ingredient, which is nontoxic to a subject. A pharmaceutically acceptable excipient includes, but is not limited to, a buffer, a stabilizer, or a preservative.

The term “package insert” is used to refer to instructions customarily included in commercial packages of therapeutic products, that contain information about the indications, usage, dosage, administration, combination therapy, contraindications and/or warnings concerning the use of such therapeutic products.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

The term “cancer” as used herein includes lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymomas, medulloblastomas, meningiomas, squamous cell carcinomas, pituitary adenoma, lymphoma, lymphocytic leukemia, including refractory versions of any of the above cancers, or a combination of one or more of the above cancers. In one embodiment, the term cancer refers to an HLA-G expressing cancer. In particular, HLA-G expressing cancers are selected renal cell carcinoma, colorectal carcinoma, non-small cell lung cancer and pancreatic duct adenocarcinoma (PDAC).

The term “expression of HLA-G” is intended to indicate a significant level of expression HLA-G in a cell, preferably on the cell surface of a cell from a tumor or cancer, preferably from a solid tumor. Patients having an “HLA-G expressing cancer” can be determined by standard assays known in the art. For example HLA-G antigen expression can be measured using immunohistochemical (IHC) detection, FACS or via PCR-based detection of the corresponding mRNA.

The term “HLA-G expressing cancer” or “HLA-G positive cancer” as used herein refers to all cancers in which the cancer cells show an expression of the HLA-G antigen. Preferably HLA-G expressing cancer as used herein refers to lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, carcinoma of the fallopian tubes, carcinoma of the endometrium, carcinoma of the cervix, carcinoma of the vagina, carcinoma of the vulva, Hodgkin's Disease, nasopharyngeal carcinoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymomas, medulloblastomas, meningiomas, squamous cell carcinomas, pituitary adenoma, lymphoma, lymphocytic leukemia, including refractory versions of any of the above cancers, or a combination of one or more of the above cancers. In particular, HLA-G expressing cancers are selected renal cell carcinoma, colorectal carcinoma, non-small cell lung cancer and pancreatic duct adenocarcinoma (PDAC).

The term “a method of treating”, “a method of treatment” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

The terms “combination”, “co-administration” or “co-administering” refer to the administration of an anti-HLA-G/anti-CD3 bispecific antibody, and an anti-PD1/anti-LAG3 bispecific antibody as two separate formulations (or as one single formulation). The co-administration can be simultaneous or sequential in either order, wherein preferably there is a time period while both (or all) active agents simultaneously exert their biological activities. The anti-HLA-G/anti-CD3 bispecific antibody and the anti-PD1/anti-LAG3 bispecific antibody are co-administered either simultaneously or sequentially (e.g. intravenously (i.v.) through a continuous infusion (one for the anti-HLA-G/anti-CD3 bispecific antibody and one for the anti-PD1/anti-LAG3 bispecific antibody). When both therapeutic agents are co-administered sequentially, the dose is administered either on the same day in two separate administrations, or one of the agents is administered on day 1 and the second is co-administered between day 2 to day 7, preferably between day 2 to 4. Thus the term “sequentially” means within 7 days after the dose of the first component (anti-HLA-G/anti-CD3 bispecific antibody or the anti-PD1/anti-LAG3 bispecific antibody), preferably within 4 days after the dose of the first component; and the term “simultaneously” means at the same time. The terms “co-administration” with respect to the maintenance doses of the anti-HLA-G/anti-CD3 bispecific antibody and the anti-PD1/anti-LAG3 bispecific antibody mean that the maintenance doses can be either co-administered simultaneously, if the treatment cycle is appropriate for both drugs, e.g. every week. Or the anti-PD1/anti-LAG3 bispecific antibody is administered e.g. every second week and the anti-HLA-G/anti-CD3 bispecific antibody is administered every third week. Or the maintenance doses are co-administered sequentially, either within one or within several days.

### Exemplary Anti-HLA-G/Anti-CD3 Bispecific Antibodies for Use in the Invention

It has been found that a combination of an anti-HLA-G/anti-CD3 bispecific antibody with an anti-PD1/anti-LAG3 bispecific antibody shows greater efficacy in a in a tumor model in humanized mice, than anti-HLAG/anti-CD3 bispecific antibody monotherapy, or a combination of an anti-HLAG/anti-CD3 bispecific antibody with an anti-PD1 antibody and an anti-LAG3 antibody. The data demonstrate that the combination with an anti-PD1/anti-LAG3 bispecific antibody is advantageous over a combination with anti-PD1 and/or anti-LAG3 monospecific antibodies as it provides better efficacy, as shown by stronger tumor growth inhibition and a better activation of T cells.

The present invention relates to anti-HLA-G/anti-CD3 bispecific antibodies and their use in combination with anti-PD1/anti-LAG3 bispecific antibodies, in particular to their use in a method for treating or delaying progression of HLA-G-expressing cancer. The anti-HLA-G/anti-CD3 bispecific antibodies as used herein are bispecific antibodies comprising a first antigen binding domain that binds to CD3, and a second antigen binding domain that binds to HLA-G. They are thus targeting HLA-G-expressing tumor cells.

Thus, the anti-HLA-G/anti-CD3 bispecific antibody as used herein comprises a first antigen binding domain comprising a heavy chain variable region (V_{H}CD3) and a light chain variable region (V_{L}CD3) specifically binding to CD3, and a second antigen binding domain comprising a heavy chain variable region (V_{H}HLA-G) and a light chain variable region (V_{L}HLA-G) specifically binding to HLA-G.

In a particular aspect, the anti-HLA-G/anti-CD3 bispecific antibody for use in the combination comprises a first antigen binding domain comprising a heavy chain variable region (V_{H}CD3) comprising CDR-H1 sequence of SEQ ID NO:41, CDR-H2 sequence of SEQ ID NO:42, and CDR-H3 sequence of SEQ ID NO:43; and/or a light chain variable region (V_{L}CD3) comprising CDR-L1 sequence of SEQ ID NO:44, CDR-L2 sequence of SEQ ID NO:45, and CDR-L3 sequence of SEQ ID NO:46. More particularly, the anti-HLA-G/anti-CD3 bispecific comprises a first antigen binding domain comprising a heavy chain variable region (V_{H}CD3) that is at least 90%, 95%, 96%, 97%, 98%, or 99% identical to the amino acid sequence of SEQ ID NO:47 and/or a light chain variable region (V_{L}CD3) that is at least 90%, 95%, 96%, 97%, 98%, or 99% identical to the amino acid sequence of SEQ ID NO:48. In a further aspect, the anti-HLA-G/anti-CD3 bispecific antibody comprises a heavy chain variable region (V_{H}CD3) comprising the amino acid sequence of SEQ ID NO:47 and/or a light chain variable region (V_{L}CD3) comprising the amino acid sequence of SEQ ID NO:48.

In another aspect, the anti-HLA-G/anti-CD3 bispecific antibody comprises a second antigen binding domain specifically binding to PD1 comprising a heavy chain variable region (V_{H}HLA-G) comprising CDR-H1 sequence of SEQ ID NO:49, CDR-H2 sequence of SEQ ID NO:50, and CDR-H3 sequence of SEQ ID NO:51, and/or a light chain variable region (V_{L}HLA-G) comprising CDR-L1 sequence of SEQ ID NO:52, CDR-L2 sequence of SEQ ID NO:53, and CDR-L3 sequence of SEQ ID NO:54. More particularly, the anti-HLA-G/anti-CD3 bispecific antibody comprises a second antigen binding domain comprising a heavy chain variable region (V_{H}HLA-G) that is at least 90%, 95%, 96%, 97%, 98%, or 99% identical to the amino acid sequence of SEQ ID NO: 55 and/or a light chain variable region (V_{L}HLA-G) that is at least 90%, 95%, 96%, 97%, 98%, or 99% identical to the amino acid sequence of SEQ ID NO:56. In a further aspect, the anti-HLA-G/anti-CD3 bispecific comprises a second antigen binding domain comprising a heavy chain variable region (V_{H}HLA-G) comprising the amino acid sequence of SEQ ID NO:55 and/or a light chain variable region (V_{L}HLA-G) comprising the amino acid sequence of SEQ ID NO:56. In one aspect, the anti-HLA-G/anti-CD3 bispecific antibody comprises a antigen binding domain specifically binding to CD3 comprising a VH domain comprising the amino acid sequence of SEQ ID NO: 47 and a VL domain comprising the amino acid sequence of SEQ ID NO:48, and a second antigen binding domain specifically binding to HLA-G comprising a VH domain comprising the amino acid sequence of SEQ ID NO:55 and a VL domain comprising the amino acid sequence of SEQ ID NO:56.

In another particular aspect, the anti-HLA-G/anti-CD3 bispecific antibody comprises a third antigen binding domain that binds to HLA-G. In particular, the anti-HLA-G/anti-CD3 bispecific antibody comprises a third antigen binding domain comprising a heavy chain variable region (V_{H}HLA-G) comprising CDR-H1 sequence of SEQ ID NO:49, CDR-H2 sequence of SEQ ID NO: 50, and CDR-H3 sequence of SEQ ID NO:51; and/or a light chain variable region (V_{L}HLA-G) comprising CDR-L1 sequence of SEQ ID NO:52, CDR-L2 sequence of SEQ ID NO:53, and CDR-L3 sequence of SEQ ID NO:54. More particularly, the anti-HLA-G/anti-CD3 bispecific antibody comprises a third antigen binding domain comprising a heavy chain variable region (V_{H}HLA-G) that is at least 90%, 95%, 96%, 97%, 98%, or 99% identical to the amino acid sequence of SEQ ID NO:55 and/or a light chain variable region (V_{L}HLA-G) that is at least 90%, 95%, 96%, 97%, 98%, or 99% identical to the amino acid sequence of SEQ ID NO:56. In a further aspect, the anti-HLA-G/anti-CD3 bispecific antibody comprises a third antigen binding domain comprising a heavy chain variable region (V_{H}HLA-G) comprising the amino acid sequence of SEQ ID NO:55 and/or a light chain variable region (V_{L}HLA-G) comprising the amino acid sequence of SEQ ID NO:56. In one aspect, the anti-HLA-G/anti-CD3 bispecific antibody comprises a antigen binding domain specifically binding to CD3 comprising a VH domain comprising the amino acid sequence of SEQ ID NO:47 and a VL domain comprising the amino acid sequence of SEQ ID NO:48, and a second, and optionally a third, antigen binding domain specifically binding to HLA-G comprising a VH domain comprising the amino acid sequence of SEQ ID NO:55 and a VL domain comprising the amino acid sequence of SEQ ID NO:56.

In one aspect, the anti-HLA-G/anti-CD3 bispecific antibody as used herein is a full-length antibody. In one aspect, the anti-HLA-G/anti-CD3 bispecific antibody as used herein is an antibody of the human IgG class, particularly an antibody of the human IgG₁ class.

In a further aspect, the anti-HLA-G/anti-CD3 bispecific antibody is a bispecific antibody wherein the first antigen binding domain is a cross-Fab molecule wherein the variable domains or the constant domains of the Fab heavy and light chain are exchanged, and the second and third, if present, antigen binding domain is a conventional Fab molecule.

The Fab molecules may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S) (SEQ ID NO:71), (G₄S)₂ or GGGGSGGGGS (SEQ ID NO:72), (G₄S)₃ (SEQ ID NO:73) and (G₄S)₄ (SEQ ID NO:74), more particularly (G₄S)₂ or GGGGSGGGGS (SEQ ID NO: 72). A particularly suitable peptide linker for fusing the Fab light chains of the first and the second Fab molecule to each other is (G₄S)₂. Another suitable linker comprises the sequence (G₄S)₄ (SEQ ID NO:74). Additionally, linkers may comprise (a portion of) an immunoglobulin hinge region. Particularly where a Fab molecule is fused to the N-terminus of an Fc domain subunit, it may be fused via an immunoglobulin hinge region or a portion thereof, with or without an additional peptide linker.

In one aspect, the anti-HLA-G/anti-CD3 bispecific antibody is a bispecific antibody, wherein (i) the second antigen binding domain is fused at the C-terminus of the Fab heavy chain to the N-terminus of the Fab heavy chain of the first antigen binding domain, the first antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain (as shown in FIG. 1A), or (ii) the first antigen binding domain is fused at the C-terminus of the Fab heavy chain to the N-terminus of the Fab heavy chain of the second antigen binding domain, the second antigen binding domain is fused at the C-terminus of the Fab heavy chain to the N-terminus of the first subunit of the Fc domain, and the third antigen binding domain is fused at the C-terminus of the Fab heavy chain to the N-terminus of the second subunit of the Fc domain.

In a further aspect, the anti-HLA-G/anti-CD3 bispecific antibody comprises an Fc domain comprising one or more amino acid substitutions that reduce binding to an Fc receptor and/or effector function. In particular, the anti-HLA-G/anti-CD3 bispecific antibody comprises an IgG1 Fc domain comprising the amino acid substitutions L234A, L235A and P329G (according to EU numbering).

In a particular aspect, the anti-HLA-G/anti-CD3 bispecific antibody comprises a first polypeptide that is at least 95%, 96%, 97%, 98%, or 99% identical to the sequence of SEQ ID NO: 57, a second polypeptide that is at least 95%, 96%, 97%, 98%, or 99% identical to the sequence of SEQ ID NO:58, a third polypeptide that is at least 95%, 96%, 97%, 98%, or 99% identical to the sequence of SEQ ID NO:59, and a fourth polypeptide that is at least 95%, 96%, 97%, 98%, or 99% identical to the sequence of SEQ ID NO:60. In a further particular embodiment, the bispecific antibody comprises a first polypeptide sequence of SEQ ID NO:57, a second polypeptide sequence of SEQ ID NO:58, a third polypeptide sequence of SEQ ID NO:59 and a fourth polypeptide sequence of SEQ ID NO:60 (HLA-G TCB).

In another aspect, the anti-HLA-G/anti-CD3 bispecific antibody for use in the combination comprises a first antigen binding domain comprising a heavy chain variable region (V_{H}CD3) comprising CDR-H1 sequence of SEQ ID NO:83, CDR-H2 sequence of SEQ ID NO:84, and CDR-H3 sequence of SEQ ID NO:85; and/or a light chain variable region (V_{L}CD3) comprising CDR-L1 sequence of SEQ ID NO:86, CDR-L2 sequence of SEQ ID NO:87, and CDR-L3 sequence of SEQ ID NO:88. More particularly, the anti-HLA-G/anti-CD3 bispecific comprises a first antigen binding domain comprising a heavy chain variable region (V_{H}CD3) that is at least 90%, 95%, 96%, 97%, 98%, or 99% identical to the amino acid sequence of SEQ ID NO:89 and/or a light chain variable region (V_{L}CD3) that is at least 90%, 95%, 96%, 97%, 98%, or 99% identical to the amino acid sequence of SEQ ID NO:90. In a further aspect, the anti-HLA-G/anti-CD3 bispecific antibody comprises a heavy chain variable region (V_{H}CD3) comprising the amino acid sequence of SEQ ID NO:89 and/or a light chain variable region (V_{L}CD3) comprising the amino acid sequence of SEQ ID NO:90.

In a further aspect, the anti-HLA-G/anti-CD3 bispecific antibody comprises a second antigen binding domain comprising a heavy chain variable region (V_{H}HLA-G) comprising CDR-H1 sequence of SEQ ID NO:91, CDR-H2 sequence of SEQ ID NO:92, and CDR-H3 sequence of SEQ ID NO: 93, and/or a light chain variable region (V_{L}HLA-G) comprising CDR-L1 sequence of SEQ ID NO: 94, CDR-L2 sequence of SEQ ID NO:95, and CDR-L3 sequence of SEQ ID NO:96. More particularly, the anti-HLA-G/anti-CD3 bispecific antibody comprises a second antigen binding domain comprising a heavy chain variable region (V_{H}HLA-G) that is at least 90%, 95%, 96%, 97%, 98%, or 99% identical to the amino acid sequence of SEQ ID NO:97 and/or a light chain variable region (V_{L}HLA-G) that is at least 90%, 95%, 96%, 97%, 98%, or 99% identical to the amino acid sequence of SEQ ID NO:98. In a further aspect, the anti-HLA-G/anti-CD3 bispecific antibody comprises a second antigen binding domain comprising a heavy chain variable region (V_{H}HLA-G) comprising the amino acid sequence of SEQ ID NO:97 and/or a light chain variable region (V_{L}HLA-G) comprising the amino acid sequence of SEQ ID NO:98.

In a particular aspect, the anti-HLA-G/anti-CD3 bispecific antibody comprises a first polypeptide that is at least 95%, 96%, 97%, 98%, or 99% identical to the sequence of SEQ ID NO: 99, a second polypeptide that is at least 95%, 96%, 97%, 98%, or 99% identical to the sequence of SEQ ID NO:100, a third polypeptide that is at least 95%, 96%, 97%, 98%, or 99% identical to the sequence of SEQ ID NO: 101, and a fourth polypeptide that is at least 95%, 96%, 97%, 98%, or 99% identical to the sequence of SEQ ID NO:102. In a further particular embodiment, the bispecific antibody comprises a first polypeptide sequence of SEQ ID NO:99, a second polypeptide sequence of SEQ ID NO: 100, a third polypeptide sequence of SEQ ID NO: 101 and a fourth polypeptide sequence of SEQ ID NO: 102.

Particular anti-HLA-G/anti-CD3 bispecific antibodies are described in PCT publication no. WO 2022/24024 or in WO 2022/129120.

In a further aspect, the anti-HLA-G/anti-CD3 bispecific antibody may also comprise a bispecific T cell engager (BiTE®).

### Exemplary Bispecific Anti-PD1/Anti-LAG3 Antibodies for Use in the Invention

For the combination provided herein, used are novel bispecific antibodies comprising a first antigen binding domain that specifically binds to to programmed cell death protein 1 (PD1) and a second antigen binding domain that specifically binds to Lymphocyte activation gene-3 (LAG3), with particularly advantageous properties such as producibility, stability, binding affinity, biological activity, specific targeting of certain T cells, targeting efficiency and reduced toxicity. Particular bispecific anti-PD1/anti-LAG3 antibodies for use herein are described in WO 2018/185043 A1.

In certain aspects, a bispecific antibody comprising a first antigen binding domain that specifically binds to PD1 and a second antigen binding domain that specifically binds to LAG3 is provided that shows reduced internalization upon binding to the T cell surface. The internalization represents an important sink for the molecule which can be degraded within a few hours while the targeted receptors are rapidly re-expressed on the cell-surface ready to inhibit TCR-signalling. In further aspects, a bispecific antibody comprising a first antigen binding domain that specifically binds to PD1 and a second antigen binding domain that specifically binds to LAG3 is provided that preferentially binds to conventional T cells rather than to Tregs. This is advantageous because targeting LAG3 on Tregs with blocking antibodies could be detrimental by increasing their suppressive function and eventually mask the positive blocking effect on other T cells. In a further aspect, a bispecific antibody comprising a first antigen binding domain that specifically binds to PD1 and a second antigen binding domain that specifically binds to LAG3 is provided that is able to rescue T cell effector functions from Treg suppression. In another aspect, a bispecific antibody comprising a first antigen binding domain that specifically binds to PD1 and a second antigen binding domain that specifically binds to LAG3 is provided that is able to induce Granzyme B secretion by CD4 T cells, when co-cultured with the tumor cell line ARH77 as shown in the assay provided herein. In a further aspect, a bispecific antibody comprising a first antigen binding domain that specifically binds to PD1 and a second antigen binding domain that specifically binds to LAG3 is provided that shows increased tumor-specific T cell effector functions and/or enhances the cytotoxic effect of T cells. In another aspect, a bispecific antibody comprising a first antigen binding domain that specifically binds to PD1 and a second antigen binding domain that specifically binds to LAG3 is provided that shows increased tumor eradication in vivo.

In one aspect, the invention provides an anti-PD1/anti-LAG3 bispecific antibody for use in the combination comprising a first antigen binding domain that specifically binds to PD1 and a second antigen binding domain that specifically binds to LAG3, wherein said first antigen binding domain specifically binding to PD1 comprises
a VH domain comprising
(i) CDR-H1 comprising the amino acid sequence of SEQ ID NO:1,
(ii) CDR-H2 comprising the amino acid sequence of SEQ ID NO:2, and
(iii) CDR-H3 comprising the amino acid sequence of SEQ ID NO:3; and
a VL domain comprising
(i) CDR-L1 comprising the amino acid sequence of SEQ ID NO:4;
(ii) CDR-L2 comprising the amino acid sequence of SEQ ID NO:5, and
(iii) CDR-L3 comprising the amino acid sequence of SEQ ID NO:6.

In one aspect, the anti-PD1/anti-LAG3 bispecific antibody comprises an Fc domain that is an IgG, particularly an IgG1 Fc domain or an IgG4 Fc domain and wherein the Fc domain has reduced or even abolished effector function. In particular, the Fc domain comprises one or more amino acid substitution that reduces binding to an Fc receptor, in particular towards Fcγ receptor.

In a further aspect, provided is a bispecific antib an anti-PD1/anti-LAG3 bispecific antibody for use in the combination comprising a first antigen binding domain that specifically binds to PD1 and a second antigen binding domain that specifically binds to LAG3, wherein the bispecific antibody comprises a Fc domain that is an IgG, particularly an IgG1 Fc domain or an IgG4 Fc domain and wherein the Fc domain comprises one or more amino acid substitution that reduces binding to an Fc receptor, in particular towards Fcγ receptor.

In another aspect, provided is an anti-PD1/anti-LAG3 bispecific antibody for use in the combination comprising a first antigen binding domain that specifically binds to PD1 and a second antigen binding domain that specifically binds to LAG3, wherein the second antigen binding domain that specifically binds to LAG3 comprises
(a) a VH domain comprising
   (i) CDR-H1 comprising the amino acid sequence of SEQ ID NO:11,
   (ii) CDR-H2 comprising the amino acid sequence of SEQ ID NO:12, and
   (iii) CDR-H3 comprising the amino acid sequence of SEQ ID NO:13; and
   a VL domain comprising
   (i) CDR-L1 comprising the amino acid sequence of SEQ ID NO:14,
   (ii) CDR-L2 comprising the amino acid sequence of SEQ ID NO: 15, and
   (iii) CDR-L3 comprising the amino acid sequence of SEQ ID NO:16; or
(b) a VH domain comprising
   (i) CDR-H1 comprising the amino acid sequence of SEQ ID NO:19,
   (ii) CDR-H2 comprising the amino acid sequence of SEQ ID NO:20, and
   (iii) CDR-H3 comprising the amino acid sequence of SEQ ID NO:21; and
   a VL domain comprising
   (i) CDR-L1 comprising the amino acid sequence of SEQ ID NO:22,
   (ii) CDR-L2 comprising the amino acid sequence of SEQ ID NO:23, and
   (iii) CDR-L3 comprising the amino acid sequence of SEQ ID NO:24.

In a further aspect, provided is an anti-PD1/anti-LAG3 bispecific antibody for use in the combination comprising a first antigen binding domain that specifically binds to PD1 and a second antigen binding domain that specifically binds to LAG3, wherein the first antigen binding domain specifically binding to PD1 comprises the VH domain comprising the amino acid sequence of SEQ ID NO: 9 and the VL domain comprising the amino acid sequence of SEQ ID NO:10.

In another aspect, provided is an anti-PD1/anti-LAG3 bispecific antibody for use in the combination comprising a first antigen binding domain that specifically binds to PD1 and a second antigen binding domain that specifically binds to LAG3, wherein the second antigen binding domain specifically binding to LAG3 comprises
(a) a VH domain comprising the amino acid sequence of SEQ ID NO:17 and a VL domain comprising the amino acid sequence of SEQ ID NO:18, or
(b) a VH domain comprising the amino acid sequence of SEQ ID NO:25 and a VL domain comprising the amino acid sequence of SEQ ID NO:26.

In a further aspect, provided is an anti-PD1/anti-LAG3 bispecific antibody for use in the combination comprising a first antigen binding domain that specifically binds to PD1 and a second antigen binding domain that specifically binds to LAG3, wherein the second antigen binding domain specifically binding to LAG3 comprises
(a) a VH domain comprising the amino acid sequence of SEQ ID NO:27 and a VL domain comprising the amino acid sequence of SEQ ID NO:28, or
(b) a VH domain comprising the amino acid sequence of SEQ ID NO:29 and a VL domain comprising the amino acid sequence of SEQ ID NO:30, or
(c) a VH domain comprising the amino acid sequence of SEQ ID NO:31 and a VL domain comprising the amino acid sequence of SEQ ID NO:32, or
(d) a VH domain comprising the amino acid sequence of SEQ ID NO:33 and a VL domain comprising the amino acid sequence of SEQ ID NO:34, or
(e) a VH domain comprising the amino acid sequence of SEQ ID NO:64 and a VL domain comprising the amino acid sequence of SEQ ID NO:65.

In another aspect, provided is an anti-PD1/anti-LAG3 bispecific antibody for use in the combination comprising a first antigen binding domain that specifically binds to PD1 and a second antigen binding domain that specifically binds to LAG3, wherein the second antigen binding domain specifically binding to LAG3 comprises a VH domain comprising the amino acid sequence of SEQ ID NO:81 and a VL domain comprising the amino acid sequence of SEQ ID NO:82.

In a particular aspect, provided is an anti-PD1/anti-LAG3 bispecific antibody for use in the combination comprising a first antigen binding domain that specifically binds to PD1 and a second antigen binding domain that specifically binds to LAG3, wherein
the first antigen binding domain specifically binding to PD1 comprises a VH domain comprising the amino acid sequence of SEQ ID NO:9 and a VL domain comprising the amino acid sequence of SEQ ID NO: 10,
and the second antigen binding domain specifically binding to LAG3 comprises a VH domain comprising the amino acid sequence of SEQ ID NO:17 and a VL domain comprising the amino acid sequence of SEQ ID NO: 18 or a VH domain comprising the amino acid sequence of SEQ ID NO:25 and a VL domain comprising the amino acid sequence of SEQ ID NO:26.

In one aspect, the bispecific antibody of the invention comprises a first antigen binding domain specifically binding to PD1 comprising a VH domain comprising the amino acid sequence of SEQ ID NO:9 and a VL domain comprising the amino acid sequence of SEQ ID NO: 10 and a second antigen binding domain specifically binding to LAG3 comprising a VH domain comprising the amino acid sequence of SEQ ID NO: 17 and a VL domain comprising the amino acid sequence of SEQ ID NO:18.

In a further aspect, the anti-PD1/anti-LAG3 bispecific antibody of the invention comprises a first antigen binding domain specifically binding to PD1 comprising a VH domain comprising the amino acid sequence of SEQ ID NO:9 and a VL domain comprising the amino acid sequence of SEQ ID NO: 10 and a second antigen binding domain specifically binding to LAG3 comprising a VH domain comprising the amino acid sequence of SEQ ID NO:25 and a VL domain comprising the amino acid sequence of SEQ ID NO:26.

In a further aspect, the anti-PD1/anti-LAG3 bispecific antibody comprising a first antigen binding domain that specifically binds to PD1 and a second antigen binding domain that specifically binds to LAG3 is a human, humanized or chimeric antibody. In particular, it is a humanized or chimeric antibody.

In one aspect, the anti-PD 1/anti-LAG3 bispecific antibody comprising a first antigen binding domain that specifically binds to PD1 and a second antigen binding domain that specifically binds to LAG3 is bivalent. This means that the bispecific antibody comprises one antigen binding domain that specifically binds to PD1 and one antigen binding domain that specifically binds to LAG3 (1+1 format).

In one aspect, provided is an anti-PD1/anti-LAG3 bispecific antibody for use in the combination comprising a first antigen binding domain that specifically binds to PD1 and a second antigen binding domain that specifically binds to LAG3, wherein the bispecific antibody comprises an Fc domain, a first Fab fragment comprising the antigen binding domain that specifically binds to PD1 and a second Fab fragment comprising the antigen binding domain that specifically binds to LAG3. In a particular aspect, in one of the Fab fragments the variable domains VL and VH are replaced by each other so that the VH domain is part of the light chain and the VL domain is part of the heavy chain. In a particular aspect, in the first Fab fragment comprising the antigen binding domain that specifically binds to PD1 the variable domains VL and VH are replaced by each other.

In a particular aspect, provided is an anti-PD1/anti-LAG3 bispecific antibody for use in the combination comprising a first antigen binding domain that specifically binds to PD1 and a second antigen binding domain that specifically binds to LAG3, wherein the bispecific antibody comprises
(a) a first heavy chain comprising an amino acid sequence with at least 95% sequence identity to the sequence of SEQ ID NO:35, a first light chain comprising an amino acid sequence with at least 95% sequence identity to the sequence of SEQ ID NO:36, a second heavy chain comprising an amino acid sequence with at least 95% sequence identity to the sequence of SEQ ID NO:37, and a second light chain comprising an amino acid sequence with at least 95% sequence identity to the sequence of SEQ ID NO:38, or
(b) a first heavy chain comprising an amino acid sequence with at least 95% sequence identity to the sequence of SEQ ID NO:35, a first light chain comprising an amino acid sequence with at least sequence 95% identity to the sequence of SEQ ID NO:36, a second heavy chain comprising an amino acid sequence with at least 95% sequence identity to the sequence of SEQ ID NO:39, and a second light chain comprising an amino acid sequence with at least 95% sequence identity to the sequence of SEQ ID NO:40.

More particularly, the bispecific antibody as used herein comprises a first heavy chain comprising an amino acid sequence of SEQ ID NO:35, a first light chain comprising an amino acid sequence of SEQ ID NO:36, a second heavy chain comprising an amino acid sequence of SEQ ID NO:37, and a second light chain comprising an amino acid sequence of SEQ ID NO:38.

Fc Domain Modifications Reducing Fc Receptor Binding and/or Effector Function

In certain aspects, provided is an anti-PD1/anti-LAG3 bispecific antibody and/or an anti-HLA-G/anti-CD3 bispecific antibody, wherein the bispecific antibody comprises an Fc domain comprising one or more amino acid modifications that reduce binding to an Fc receptor, in particular towards Fcγ receptor, and reduce or abolish effector function.

In certain aspects, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

The following section describes preferred aspects of the bispecific antigen binding molecules of the invention comprising Fc domain modifications reducing Fc receptor binding and/or effector function. In one aspect, the invention relates to an anti-PD1/anti-LAG3 bispecific antibody and/or an anti-HLA-G/anti-CD3 bispecific antibody, wherein the Fc domain comprises one or more amino acid substitution that reduces binding to an Fc receptor, in particular towards Fcγ receptor. In particular, the Fc domain is of human IgG1 subclass with the amino acid mutations L234A, L235A and P329G (numbering according to Kabat EU index).

The Fc domain confers favorable pharmacokinetic properties to the bispecific antibodies of the invention, including a long serum half-life which contributes to good accumulation in the target tissue and a favorable tissue-blood distribution ratio. At the same time it may, however, lead to undesirable targeting of the bispecific antibodies of the invention to cells expressing Fc receptors rather than to the preferred antigen-bearing cells. Accordingly, in particular embodiments the Fc domain of the bispecific antibodies of the invention exhibits reduced binding affinity to an Fc receptor and/or reduced effector function, as compared to a native IgG Fc domain, in particular an IgG1 Fc domain or an IgG4 Fc domain. More particularly, the Fc domain is an IgG1 FC domain.

In one such aspect the Fc domain (or the bispecific antigen binding molecule of the invention comprising said Fc domain) exhibits less than 50%, preferably less than 20%, more preferably less than 10% and most preferably less than 5% of the binding affinity to an Fc receptor, as compared to a native IgG1 Fc domain (or the bispecific antigen binding molecule of the invention comprising a native IgG1 Fc domain), and/or less than 50%, preferably less than 20%, more preferably less than 10% and most preferably less than 5% of the effector function, as compared to a native IgG1 Fc domain (or the bispecific antigen binding molecule of the invention comprising a native IgG1 Fc domain). In one aspect, the Fc domain (or the bispecific antigen binding molecule of the invention comprising said Fc domain) does not substantially bind to an Fc receptor and/or induce effector function. In a particular aspect the Fc receptor is an Fcγ receptor. In one aspect, the Fc receptor is a human Fc receptor. In one aspect, the Fc receptor is an activating Fc receptor. In a specific aspect, the Fc receptor is an activating human Fcγ receptor, more specifically human FcγRIIIa, FcγRI or FcγRIIa, most specifically human FcγRIIIa. In one aspect, the Fc receptor is an inhibitory Fc receptor. In a specific aspect, the Fc receptor is an inhibitory human Fcγ receptor, more specifically human FcγRIIB. In one aspect the effector function is one or more of CDC, ADCC, ADCP, and cytokine secretion. In a particular aspect, the effector function is ADCC. In one aspect, the Fc domain domain exhibits substantially similar binding affinity to neonatal Fc receptor (FcRn), as compared to a native IgG1 Fc domain. Substantially similar binding to FcRn is achieved when the Fc domain (or the bispecific antigen binding molecule of the invention comprising said Fc domain) exhibits greater than about 70%, particularly greater than about 80%, more particularly greater than about 90% of the binding affinity of a native IgG1 Fc domain (or the bispecific antigen binding molecule of the invention comprising a native IgG1 Fc domain) to FcRn.

In a particular aspect, the Fc domain is engineered to have reduced binding affinity to an Fc receptor and/or reduced effector function, as compared to a non-engineered Fc domain. In a particular aspect, the Fc domain of the bispecific antigen binding molecule of the invention comprises one or more amino acid mutation that reduces the binding affinity of the Fc domain to an Fc receptor and/or effector function. Typically, the same one or more amino acid mutation is present in each of the two subunits of the Fc domain. In one aspect, the amino acid mutation reduces the binding affinity of the Fc domain to an Fc receptor. In another aspect, the amino acid mutation reduces the binding affinity of the Fc domain to an Fc receptor by at least 2-fold, at least 5-fold, or at least 10-fold. In one aspect, the bispecific antigen binding molecule of the invention comprising an engineered Fc domain exhibits less than 20%, particularly less than 10%, more particularly less than 5% of the binding affinity to an Fc receptor as compared to bispecific antibodies of the invention comprising a non-engineered Fc domain. In a particular aspect, the Fc receptor is an Fcγ receptor. In other aspects, the Fc receptor is a human Fc receptor. In one aspect, the Fc receptor is an inhibitory Fc receptor. In a specific aspect, the Fc receptor is an inhibitory human Fcγ receptor, more specifically human FcγRIIB. In some aspects the Fc receptor is an activating Fc receptor. In a specific aspect, the Fc receptor is an activating human Fcγ receptor, more specifically human FcγRIIIa, FcγRI or FcγRIIa, most specifically human FcγRIIIa.

Preferably, binding to each of these receptors is reduced. In some aspects, binding affinity to a complement component, specifically binding affinity to Clq, is also reduced. In one aspect, binding affinity to neonatal Fc receptor (FcRn) is not reduced. Substantially similar binding to FcRn. i.e. preservation of the binding affinity of the Fc domain to said receptor, is achieved when the Fc domain (or the bispecific antigen binding molecule of the invention comprising said Fc domain) exhibits greater than about 70% of the binding affinity of a non-engineered form of the Fc domain (or the bispecific antigen binding molecule of the invention comprising said non-engineered form of the Fc domain) to FcRn. The Fc domain, or the bispecific antigen binding molecule of the invention comprising said Fc domain, may exhibit greater than about 80% and even greater than about 90% of such affinity. In certain embodiments the Fc domain of the bispecific antigen binding molecule of the invention is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced dendritic cell maturation, or reduced T cell priming.

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581). Certain antibody variants with improved or diminished binding to FcRs are described. (e.g. U.S. Pat. No. 6,737,056; WO 2004/056312, and Shields, R. L. et al., J. Biol. Chem. 276 (2001) 6591-6604).

In one aspect of the invention, the Fc domain comprises an amino acid substitution at a position of E233, L234, L235, N297, P331 and P329. In some aspects, the Fc domain comprises the amino acid substitutions L234A and L235A (“LALA”). In one such embodiment, the Fc domain is an IgG1 Fc domain, particularly a human IgG1 Fc domain. In one aspect, the Fc domain comprises an amino acid substitution at position P329. In a more specific aspect, the amino acid substitution is P329A or P329G, particularly P329G. In one embodiment the Fc domain comprises an amino acid substitution at position P329 and a further amino acid substitution selected from the group consisting of E233P, L234A, L235A, L235E, N297A, N297D or P331S. In more particular embodiments the Fc domain comprises the amino acid mutations L234A, L235A and P329G (“P329G LALA”). The “P329G LALA” combination of amino acid substitutions almost completely abolishes Fcγ receptor binding of a human IgG1 Fc domain, as described in PCT Patent Application No. WO 2012/130831 A1. Said document also describes methods of preparing such mutant Fc domains and methods for determining its properties such as Fc receptor binding or effector functions, such antibody is an IgG1 with mutations L234A and L235A or with mutations L234A, L235A and P329G (numbering according to EU index of Kabat et al, Kabat et al., Sequences of Proteins of Immunological Interest, 5th Ed. Public Health Service, National Institutes of Health, Bethesda, MD, 1991).

In one aspect, the anti-PD1/anti-LAG3 bispecific antibody and/or the anti-HLA-G/anti-CD3 bispecific antibody comprises (all positions according to EU index of Kabat) (i) a homodimeric Fc-region of the human IgG1 subclass optionally with the mutations P329G, L234A and L235A. or (ii) a homodimeric Fc-region of the human IgG4 subclass optionally with the mutations P329G, S228P and L235E, or (iii) a homodimeric Fc-region of the human IgG1 subclass optionally with the mutations P329G, L234A, L235A, I253A, H310A, and H435A, or optionally with the mutations P329G, L234A, L235A, H310A, H433A, and Y436A, or (iv) a heterodimeric Fc-region wherein one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C, or (v) a heterodimeric Fc-region of the human IgG1 subclass wherein both Fc-region polypeptides comprise the mutations P329G, L234A and L235A and one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C, or wherein one Fc-region polypeptide comprises the mutations T366W and S354C, and the other Fc-region polypeptide comprises the mutations T366S, L368A, Y407V and Y349C.

In one aspect, the Fc domain is an IgG4 Fc domain. In a more specific embodiment, the Fc domain is an IgG4 Fc domain comprising an amino acid substitution at position S228 (Kabat numbering), particularly the amino acid substitution S228P. In a more specific embodiment, the Fc domain is an IgG4 Fc domain comprising amino acid substitutions L235E and S228P and P329G. This amino acid substitution reduces in vivo Fab arm exchange of IgG4 antibodies (see Stubenrauch et al., Drug Metabolism and Disposition 38, 84-91 (2010)). Thus, in one aspect, provided is a bispecific antibody, comprising (all positions according to EU index of Kabat) a heterodimeric Fc-region of the human IgG4 subclass wherein both Fc-region polypeptides comprise the mutations P329G, S228P and L235E and one Fc-region polypeptide comprises the mutation T366W, and the other Fc-region polypeptide comprises the mutations T366S, L368A and Y407V, or wherein one Fc-region polypeptide comprises the mutations T366W and Y349C, and the other Fc-region polypeptide comprises the mutations T366S, L368A, Y407V, and S354C. or wherein one Fc-region polypeptide comprises the mutations T366W and S354C, and the other Fc-region polypeptide comprises the mutations T366S, L368A, Y407V and Y349C.

Antibodies with increased half lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). See also Duncan, A. R. and Winter, G, Nature 322 (1988) 738-740; U.S. Pat. Nos. 5,648,260; 5,624,821; and WO 94/29351 concerning other examples of Fc region variants.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing Fcγ IIIa receptor. Effector function of an Fc domain, or bispecific antibodies of the invention comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, CA); and CytoTox 96® non-radioactive cytotoxicity assay (Promega, Madison, WI)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

The following section describes preferred aspects of the bispecific antibodies of the invention comprising Fc domain modifications reducing Fc receptor binding and/or effector function. In one aspect, provided is an anti-PD1/anti-LAG3 bispecific antibody and/or an anti-HLA-G/anti-CD3 bispecific antibody, wherein the Fc domain comprises one or more amino acid substitution that reduces the binding affinity of the antibody to an Fc receptor, in particular towards Fcγ receptor. In another aspect, provided is an anti-PD1/anti-LAG3 bispecific antibody and/or an anti-HLA-G/anti-CD3 bispecific antibody, wherein the Fc domain comprises one or more amino acid substitution that reduces effector function. In particular aspect, the Fc domain is of human IgG1 subclass with the amino acid mutations L234A, L235A and P329G (numbering according to Kabat EU index).

### Fc Domain Modifications Promoting Heterodimerization

The bispecific antigen binding molecules as described herein comprise different antigen binding domains, fused to one or the other of the two subunits of the Fc domain, thus the two subunits of the Fc domain may be comprised in two non-identical polypeptide chains. Recombinant co-expression of these polypeptides and subsequent dimerization leads to several possible combinations of the two polypeptides. To improve the yield and purity of the bispecific antibodies of the invention in recombinant production, it will thus be advantageous to introduce in the Fc domain of the bispecific antigen binding molecules described herein a modification promoting the association of the desired polypeptides.

Accordingly, in particular aspects provided is an anti-PD1/anti-LAG3 bispecific antibody and/or an anti-HLA-G/anti-CD3 antibody, wherein the Fc domain comprises a modification promoting the association of the first and second subunit of the Fc domain. The site of most extensive protein-protein interaction between the two subunits of a human IgG Fc domain is in the CH3 domain of the Fc domain. Thus, in one aspect said modification is in the CH3 domain of the Fc domain.

In a specific aspect said modification is a so-called “knob-into-hole” modification, comprising a “knob” modification in one of the two subunits of the Fc domain and a “hole” modification in the other one of the two subunits of the Fc domain. Thus, the invention relates to a bispecific antibody comprising a first antigen binding domain that specifically binds to PD1 and a second antigen-binding site that specifically binds to LAG3 and/or a bispecific antibody comprising a first antigen binding domain that specifically binds to CD3 and a second antigen-binding site that specifically binds to HLA-G, wherein the first subunit of the Fc domain comprises knobs and the second subunit of the Fc domain comprises holes according to the knobs into holes method. In a particular aspect, the first subunit of the Fc domain comprises the amino acid substitutions S354C and T366W (EU numbering) and the second subunit of the Fc domain comprises the amino acid substitutions Y349C, T366S and Y407V (numbering according to Kabat EU index).

The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in one aspect, in the CH3 domain of the first subunit of the Fc domain of the bispecific antigen binding molecules of the invention an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable. The protuberance and cavity can be made by altering the nucleic acid encoding the polypeptides, e.g. by site-specific mutagenesis, or by peptide synthesis. In a specific aspect, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one aspect, in the second subunit of the Fc domain additionally the threonine residue at position 366 is replaced with a serine residue (T366S) and the leucine residue at position 368 is replaced with an alanine residue (L368A).

In yet a further aspect, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues leads to the formation of a disulfide bridge between the two subunits of the Fc domain, further stabilizing the dimer (Carter (2001), J Immunol Methods 248, 7-15). In a particular aspect, the first subunit of the Fc domain comprises the amino acid substitutions S354C and T366W (EU numbering) and the second subunit of the Fc domain comprises the amino acid substitutions Y349C. T366S and Y407V (numbering according to Kabat EU index).

But also other knobs-in-holes technologies as described by EP 1 870 459, can be used alternatively or additionally. In one embodiment the multispecific antibody comprises the mutations R409D and K370E in the CH3 domain of the “knobs chain” and the mutations D399K and E357K in the CH3 domain of the “hole-chain” (numbering according to Kabat EU index).

In one aspect, the bispecific antibody comprises a T366W mutation in the CH3 domain of the “knobs chain” and the mutations T366S, L368A and Y407V in the CH3 domain of the “hole chain” and additionally the mutations R409D and K370E in the CH3 domain of the “knobs chain” and the mutations D399K and E357K in the CH3 domain of the “hole chain” (numbering according to the Kabat EU index).

In one aspect, the bispecific antibody comprises the mutations Y349C and T366W in one of the two CH3 domains and the mutations S354C, T366S, L368A and Y407V in the other of the two CH3 domains, or the multispecific antibody comprises the mutations Y349C and T366W in one of the two CH3 domains and the mutations S354C, T366S, L368A and Y407V in the other of the two CH3 domains and additionally the mutations R409D and K370E in the CH3 domain of the “knobs chain” and the mutations D399K and E357K in the CH3 domain of the “hole chain” (numbering according to the Kabat EU index).

In an alternative aspect, a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

Apart from the “knob-into-hole technology” other techniques for modifying the CH3 domains of the heavy chains of a multispecific antibody to enforce heterodimerization are known in the art. These technologies, especially the ones described in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954 and WO 2013/096291 are contemplated herein as alternatives to the “knob-into-hole technology” in combination with a bispecific antibody.

In one aspect, in the bispecific antibody the approach described in EP 1870459 is used to support heterodimerization of the first heavy chain and the second heavy chain of the multispecific antibody. This approach is based on the introduction of charged amino acids with opposite charges at specific amino acid positions in the CH3/CH3-domain-interface between both, the first and the second heavy chain.

Accordingly, in this aspect in the tertiary structure of the multispecific antibody the CH3 domain of the first heavy chain and the CH3 domain of the second heavy chain form an interface that is located between the respective antibody CH3 domains, wherein the respective amino acid sequences of the CH3 domain of the first heavy chain and the amino acid sequence of the CH3 domain of the second heavy chain each comprise a set of amino acids that is located within said interface in the tertiary structure of the antibody, wherein from the set of amino acids that is located in the interface in the CH3 domain of one heavy chain a first amino acid is substituted by a positively charged amino acid and from the set of amino acids that is located in the interface in the CH3 domain of the other heavy chain a second amino acid is substituted by a negatively charged amino acid. The bispecific antibody according to this aspect is herein also referred to as “CH3 (+/−)-engineered bispecific antibody” (wherein the abbreviation “+/−” stands for the oppositely charged amino acids that were introduced in the respective CH3 domains).

In one aspect, in the CH3 (+/−)-engineered bispecific antibody the positively charged amino acid is selected from K, R and H, and the negatively charged amino acid is selected from E or D.

In one aspect, in the CH3 (+/−)-engineered bispecific antibody the positively charged amino acid is selected from K and R, and the negatively charged amino acid is selected from E or D.

In one aspect, in the CH3 (+/−)-engineered bispecific antibody the positively charged amino acid is K, and the negatively charged amino acid is E.

In one aspect, in the CH3 (+/−)-engineered bispecific antibody in the CH3 domain of one heavy chain the amino acid R at position 409 is substituted by D and the amino acid K at position is substituted by E, and in the CH3 domain of the other heavy chain the amino acid D at position 399 is substituted by K and the amino acid E at position 357 is substituted by K (numbering according to Kabat EU index).

In one aspect, the approach described in WO 2013/157953 is used to support heterodimerization of the first heavy chain and the second heavy chain of the multispecific antibody. In one embodiment in the CH3 domain of one heavy chain the amino acid T at position 366 is substituted by K, and in the CH3 domain of the other heavy chain the amino acid L at position 351 is substituted by D (numbering according to Kabat EU index). In another embodiment in the CH3 domain of one heavy chain the amino acid T at position 366 is substituted by K and the amino acid L at position 351 is substituted by K, and in the CH3 domain of the other heavy chain the amino acid L at position 351 is substituted by D (numbering according to Kabat EU index).

In another aspect, in the CH3 domain of one heavy chain the amino acid T at position 366 is substituted by K and the amino acid L at position 351 is substituted by K, and in the CH3 domain of the other heavy chain the amino acid L at position 351 is substituted by D (numbering according to Kabat EU index). Additionally at least one of the following substitutions is comprised in the CH3 domain of the other heavy chain: the amino acid Y at position 349 is substituted by E, the amino acid Y at position 349 is substituted by D and the amino acid L at position 368 is substituted by E (numbering according to Kabat EU index). In one embodiment the amino acid L at position 368 is substituted by E (numbering according to Kabat EU index).

In one aspect, the approach described in WO 2012/058768 is used to support heterodimerization of the first heavy chain and the second heavy chain of the multispecific antibody. In one aspect, in the CH3 domain of one heavy chain the amino acid L at position 351 is substituted by Y and the amino acid Y at position 407 is substituted by A, and in the CH3 domain of the other heavy chain the amino acid T at position 366 is substituted by A and the amino acid K at position 409 is substituted by F (numbering according to Kabat EU index). In another embodiment, in addition to the aforementioned substitutions, in the CH3 domain of the other heavy chain at least one of the amino acids at positions 411 (originally T), 399 (originally D), 400 (originally S), 405 (originally F), 390 (originally N) and 392 (originally K) is substituted (numbering according to Kabat EU index). Preferred substitutions are:
substituting the amino acid T at position 411 by an amino acid selected from N, R, Q, K, D, E and W (numbering according to Kabat EU index),
substituting the amino acid D at position 399 by an amino acid selected from R, W, Y, and K (numbering according to Kabat EU index),
substituting the amino acid S at position 400 by an amino acid selected from E, D, R and K (numbering according to Kabat EU index),
substituting the amino acid F at position 405 by an amino acid selected from I, M, T. S. V and W (numbering according to Kabat EU index;
substituting the amino acid N at position 390 by an amino acid selected from R. K and D (numbering according to Kabat EU index; and
substituting the amino acid K at position 392 by an amino acid selected from V, M. R. L. F and E (numbering according to Kabat EU index).

In another aspect, the bispecific antibody is engineered according to WO 2012/058768), i.e. in the CH3 domain of one heavy chain the amino acid L at position 351 is substituted by Y and the amino acid Y at position 407 is substituted by A, and in the CH3 domain of the other heavy chain the amino acid T at position 366 is substituted by V and the amino acid K at position 409 is substituted by F (numbering according to Kabat EU index). In another embodiment of the multispecific antibody, in the CH3 domain of one heavy chain the amino acid Y at position 407 is substituted by A, and in the CH3 domain of the other heavy chain the amino acid T at position 366 is substituted by A and the amino acid K at position 409 is substituted by F (numbering according to Kabat EU index). In the last aforementioned embodiment, in the CH3 domain of the other heavy chain the amino acid K at position 392 is substituted by E, the amino acid T at position 411 is substituted by E, the amino acid D at position 399 is substituted by R and the amino acid S at position 400 is substituted by R (numbering according to Kabat EU index).

In one aspect, the approach described in WO 2011/143545 is used to support heterodimerization of the first heavy chain and the second heavy chain of the multispecific antibody. In one aspect, amino acid modifications in the CH3 domains of both heavy chains are introduced at positions 368 and/or 409 (numbering according to Kabat EU index).

In one aspect, the approach described in WO 2011/090762 is used to support heterodimerization of the first heavy chain and the second heavy chain of the bispecific antibody. WO 2011/090762 relates to amino acid modifications according to the “knob-into-hole” (KiH) technology. In one embodiment in the CH3 domain of one heavy chain the amino acid T at position 366 is substituted by W, and in the CH3 domain of the other heavy chain the amino acid Y at position 407 is substituted by A (numbering according to Kabat EU index). In another embodiment in the CH3 domain of one heavy chain the amino acid T at position 366 is substituted by Y, and in the CH3 domain of the other heavy chain the amino acid Y at position 407 is substituted by T (numbering according to Kabat EU index).

In one aspect, the approach described in WO 2009/089004 is used to support heterodimerization of the first heavy chain and the second heavy chain of the bispecific antibody. In one embodiment in the CH3 domain of one heavy chain the amino acid K or N at position 392 is substituted by a negatively charged amino acid (in one embodiment by E or D, in one preferred embodiment by D), and in the CH3 domain of the other heavy chain the amino acid D at position 399 the amino acid E or D at position 356 or the amino acid E at position 357 is substituted by a positively charged amino acid (in one embodiment K or R, in one preferred embodiment by K, in one preferred embodiment the amino acids at positions 399 or 356 are substituted by K) (numbering according to Kabat EU index). In one further embodiment, in addition to the aforementioned substitutions, in the CH3 domain of the one heavy chain the amino acid K or R at position 409 is substituted by a negatively charged amino acid (in one embodiment by E or D, in one preferred embodiment by D) (numbering according to Kabat EU index). In one even further aspect, in addition to or alternatively to the aforementioned substitutions, in the CH3 domain of the one heavy chain the amino acid K at position 439 and/or the amino acid K at position 370 is substituted independently from each other by a negatively charged amino acid (in one embodiment by E or D, in one preferred embodiment by D) (numbering according to Kabat EU index).

In one aspect, the approach described in WO 2007/147901 is used to support heterodimerization of the first heavy chain and the second heavy chain of the multispecific antibody. In one embodiment in the CH3 domain of one heavy chain the amino acid K at position 253 is substituted by E, the amino acid D at position 282 is substituted by K and the amino acid K at position 322 is substituted by D, and in the CH3 domain of the other heavy chain the amino acid D at position 239 is substituted by K, the amino acid E at position 240 is substituted by K and the amino acid K at position 292 is substituted by D (numbering according to Kabat EU index).

The C-terminus of the heavy chain of the bispecific antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG.

In one aspect of all aspects as reported herein, a bispecific antibody comprising a heavy chain including a C-terminal CH3 domain as specified herein, comprises the C-terminal glycine-lysine dipeptide (G446 and K447, numbering according to Kabat EU index). In one embodiment of all aspects as reported herein, a bispecific antibody comprising a heavy chain including a C-terminal CH3 domain, as specified herein, comprises a C-terminal glycine residue (G446, numbering according to Kabat EU index).

### Modifications in the Fab Domains

In one aspect, provided is an anti-PD1/anti-LAG3 bispecific antibody and/or an anti-HLA-G/anti-CD3 antibody, wherein in one of the Fab fragments either the variable domains VH and VL or the constant domains CH1 and CL are exchanged. The bispecific antibodies are prepared according to the Crossmab technology.

Multispecific antibodies with a domain replacement/exchange in one binding arm (CrossMabVH-VL or CrossMabCH-CL) are described in detail in WO2009/080252, WO2009/080253 and Schaefer, W. et al, PNAS, 108 (2011) 11187-1191. They clearly reduce the byproducts caused by the mismatch of a light chain against a first antigen with the wrong heavy chain against the second antigen (compared to approaches without such domain exchange).

In a particular aspect, provided is an anti-PD1/anti-LAG3 bispecific antibody and/or an anti-HLA-G/anti-CD3 antibody, wherein in one of the Fab fragments the variable domains VL and VH are replaced by each other so that the VH domain is part of the light chain and the VL domain is part of the heavy chain. In a particular aspect, the bispecific antibody is one, wherein in the first Fab fragment comprising the antigen binding domain that specifically binds to PD1 the variable domains VL and VH are replaced by each other.

In another aspect, and to further improve correct pairing, the anti-PD1/anti-LAG3 bispecific antibody and/or an anti-HLA-G/anti-CD3 antibody can contain different charged amino acid substitutions (so-called “charged residues”). These modifications are introduced in the crossed or non-crossed CH1 and CL domains. Such modifiactions are described e.g. in WO2015/150447, WO2016/020309 and PCT/EP2016/073408.

In a particular aspect, provided is an anti-PD1/anti-LAG3 bispecific antibody and/or an anti-HLA-G/anti-CD3 antibody, wherein in one of the Fab fragments in the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index). In a particular aspect, the bispecific antibody is one, wherein in the second Fab fragment comprising the antigen binding domain that specifically binds to TIM3 the constant domain CL the amino acid at position 124 is substituted independently by lysine (K), arginine (R) or histidine (H) (numbering according to Kabat EU Index), and in the constant domain CH1 the amino acids at positions 147 and 213 are substituted independently by glutamic acid (E) or aspartic acid (D) (numbering according to Kabat EU index).

In a particular aspect, provided is an anti-PD1/anti-LAG3 bispecific antibody and/or an anti-HLA-G/anti-CD3 antibody, wherein in one of CL domains the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E). In a particular aspect, the bispecific antibody is one, wherein in the Fab fragment comprising the antigen binding domain that specifically binds to LAG3 the amino acid at position 123 (EU numbering) has been replaced by arginine (R) and the amino acid at position 124 (EU numbering) has been substituted by lysine (K) and wherein in one of the CH1 domains the amino acids at position 147 (EU numbering) and at position 213 (EU numbering) have been substituted by glutamic acid (E).

In a further aspect, the bispecific antibody is a bivalent antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other.

The antibody under a) does not contain a modification as reported under b) and the heavy chain and the light chain under a) are isolated chains.

In the antibody under b) within the light chain the variable light chain domain VL is replaced by the variable heavy chain domain VH of said antibody, and within the heavy chain the variable heavy chain domain VH is replaced by the variable light chain domain VL of said antibody.

In one aspect, (i) in the constant domain CL of the first light chain under a) the amino acid at position 124 (numbering according to Kabat) is substituted by a positively charged amino acid, and wherein in the constant domain CH1 of the first heavy chain under a) the amino acid at position 147 or the amino acid at position 213 (numbering according to Kabat EU index) is substituted by a negatively charged amino acid, or (ii) in the constant domain CL of the second light chain under b) the amino acid at position 124 (numbering according to Kabat) is substituted by a positively charged amino acid, and wherein in the constant domain CH1 of the second heavy chain under b) the amino acid at position 147 or the amino acid at position 213 (numbering according to Kabat EU index) is substituted by a negatively charged amino acid.

In another aspect, (i) in the constant domain CL of the first light chain under a) the amino acid at position 124 is substituted independently by lysine (K), arginine (R) or histidine (H) (numbering according to Kabat) (in one preferred embodiment independently by lysine (K) or arginine (R)), and wherein in the constant domain CH1 of the first heavy chain under a) the amino acid at position 147 or the amino acid at position 213 is substituted independently by glutamic acid (E) or aspartic acid (D) (numbering according to Kabat EU index), or (ii) in the constant domain CL of the second light chain under b) the amino acid at position 124 is substituted independently by lysine (K), arginine (R) or histidine (H) (numbering according to Kabat) (in one preferred embodiment independently by lysine (K) or arginine (R)), and wherein in the constant domain CH1 of the second heavy chain under b) the amino acid at position 147 or the amino acid at position 213 is substituted independently by glutamic acid (E) or aspartic acid (D) (numbering according to Kabat EU index).

In one aspect, in the constant domain CL of the second heavy chain the amino acids at position 124 and 123 are substituted by K (numbering according to Kabat EU index).

In one aspect, in the constant domain CL of the second heavy chain the amino acid at position 123 is substituted by R and the amino acid as position 124 is substituted by K (numbering according to Kabat EU index).

In one aspect, in the constant domain CH1 of the second light chain the amino acids at position 147 and 213 are substituted by E (numbering according to EU index of Kabat).

In one aspect, in the constant domain CL of the first light chain the amino acids at position 124 and 123 are substituted by K, and in the constant domain CH1 of the first heavy chain the amino acids at position 147 and 213 are substituted by E (numbering according to Kabat EU index).

In one aspect, in the constant domain CL of the first light chain the amino acid at position 123 is substituted by R and the amino acid at position 124 is substituted by K, and in the constant domain CH1 of the first heavy chain the amino acids at position 147 and 213 are both substituted by E (numbering according to Kabat EU index).

In one aspect, in the constant domain CL of the second heavy chain the amino acids at position 124 and 123 are substituted by K, and wherein in the constant domain CH1 of the second light chain the amino acids at position 147 and 213 are substituted by E, and in the variable domain VL of the first light chain the amino acid at position 38 is substituted by K, in the variable domain VH of the first heavy chain the amino acid at position 39 is substituted by E, in the variable domain VL of the second heavy chain the amino acid at position 38 is substituted by K, and in the variable domain VH of the second light chain the amino acid at position 39 is substituted by E (numbering according to Kabat EU index).

In one aspect, the bispecific antibody is a bivalent antibody comprising
a) a first light chain and a first heavy chain of an antibody specifically binding to a first antigen, and
b) a second light chain and a second heavy chain of an antibody specifically binding to a second antigen, wherein the variable domains VL and VH of the second light chain and the second heavy chain are replaced by each other, and wherein the constant domains CL and CH1 of the second light chain and the second heavy chain are replaced by each other.

The antibody under a) does not contain a modification as reported under b) and the heavy chain and the light chain und a) are isolated chains. In the antibody under b) within the light chain the variable light chain domain VL is replaced by the variable heavy chain domain VH of said antibody, and the constant light chain domain CL is replaced by the constant heavy chain domain CH1 of said antibody; and within the heavy chain the variable heavy chain domain VH is replaced by the variable light chain domain VL of said antibody, and the constant heavy chain domain CH1 is replaced by the constant light chain domain CL of said antibody.

In one aspect, the bispecific antibody is a bivalent antibody comprising
a) a first light chain and a first heavy chain of an antibody specifically binding to a first antigen, and
b) a second light chain and a second heavy chain of an antibody specifically binding to a second antigen, wherein the constant domains CL and CH1 of the second light chain and the second heavy chain are replaced by each other.

The antibody under a) does not contain a modification as reported under b) and the heavy chain and the light chain under a) are isolated chains. In the antibody under b) within the light chain the constant light chain domain CL is replaced by the constant heavy chain domain CHIof said antibody; and within the heavy chain the constant heavy chain domain CH1 is replaced by the constant light chain domain CL of said antibody.

In one aspect, the bispecific antibody is a bispecific antibody comprising
a) a full length antibody specifically binding to a first antigen and consisting of two antibody heavy chains and two antibody light chains, and
b) one, two, three or four single chain Fab fragments specifically binding to a second antigen,
wherein said single chain Fab fragments under b) are fused to said full length antibody under a) via a peptide linker at the C- or N-terminus of the heavy or light chain of said full length antibody.

In one aspect, one or two identical single chain Fab fragments binding to a second antigen are fused to the full length antibody via a peptide linker at the C terminus of the heavy or light chains of said full length antibody.

In one aspect, one or two identical single chain Fab (scFab) fragments binding to a second antigen are fused to the full length antibody via a peptide linker at the C terminus of the heavy chains of said full length antibody.

In one aspect, one or two identical single chain Fab (scFab) fragments binding to a second antigen are fused to the full length antibody via a peptide linker at the C terminus of the light chains of said full length antibody.

In one aspect, two identical single chain Fab (scFab) fragments binding to a second antigen are fused to the full length antibody via a peptide linker at the C-terminus of each heavy or light chain of said full length antibody.

In one aspect, two identical single chain Fab (scFab) fragments binding to a second antigen are fused to the full length antibody via a peptide linker at the C-terminus of each heavy chain of said full length antibody.

In one aspect, two identical single chain Fab (scFab) fragments binding to a second antigen are fused to the full length antibody via a peptide linker at the C-terminus of each light chain of said full length antibody.

In one aspect, the bispecific antibody is a trivalent antibody comprising
a) a full length antibody specifically binding to a first antigen and consisting of two antibody heavy chains and two antibody light chains,
b) a first polypeptide consisting of
   ba) an antibody heavy chain variable domain (VH), or
   bb) an antibody heavy chain variable domain (VH) and an antibody constant domain 1 (CH1),
wherein said first polypeptide is fused with the N-terminus of its VH domain via a peptidic linker to the C-terminus of one of the two heavy chains of said full length antibody,
c) a second polypeptide consisting of
   ca) an antibody light chain variable domain (VL), or
   cb) an antibody light chain variable domain (VL) and an antibody light chain constant domain (CL),
wherein said second polypeptide is fused with the N-terminus of the VL domain via a peptide linker to the C-terminus of the other of the two heavy chains of said full length antibody, and
wherein the antibody heavy chain variable domain (VH) of the first polypeptide and the antibody light chain variable domain (VL) of the second polypeptide together form an antigen binding domain specifically binding to a second antigen.

In one aspect, the antibody heavy chain variable domain (VH) of the polypeptide under b) and the antibody light chain variable domain (VL) of the polypeptide under c) are linked and stabilized via an interchain disulfide bridge by introduction of a disulfide bond between the following positions:
(i) heavy chain variable domain position 44 to light chain variable domain position 100, or
(ii) heavy chain variable domain position 105 to light chain variable domain position 43, or
(iii) heavy chain variable domain position 101 to light chain variable domain position 100 (numbering always according to Kabat EU index).

Techniques to introduce unnatural disulfide bridges for stabilization are described e.g. in WO 94/029350, Rajagopal, V., et al., Prot. Eng. (1997) 1453-1459; Kobayashi, H., et al., Nucl. Med. Biol. 25 (1998) 387-393; and Schmidt, M., et al., Oncogene 18 (1999) 1711-1721. In one embodiment the optional disulfide bond between the variable domains of the polypeptides under b) and c) is between heavy chain variable domain position 44 and light chain variable domain position 100. In one embodiment the optional disulfide bond between the variable domains of the polypeptides under b) and c) is between heavy chain variable domain position 105 and light chain variable domain position 43 (numbering always according to Kabat). In one embodiment a trivalent, bispecific antibody without said optional disulfide stabilization between the variable domains VH and VL of the single chain Fab fragments is preferred.

In one aspect, the bispecific antibody is a trispecific or tetraspecific antibody, comprising
a) a first light chain and a first heavy chain of a full length antibody which specifically binds to a first antigen, and
b) a second (modified) light chain and a second (modified) heavy chain of a full length antibody which specifically binds to a second antigen, wherein the variable domains VL and VH are replaced by each other, and/or wherein the constant domains CL and CH1 are replaced by each other, and
c) wherein one to four antigen binding domains which specifically bind to one or two further antigens (i.e. to a third and/or fourth antigen) are fused via a peptide linker to the C- or N-terminus of the light chains or heavy chains of a) and/or b).

The antibody under a) does not contain a modification as reported under b) and the heavy chain and the light chain und a) are isolated chains.

In one aspect, the trispecific or tetraspecific antibody comprises under c) one or two antigen binding domains which specifically bind to one or two further antigens.

In one aspect, the antigen binding domains are selected from the group of a scFv fragment and a scFab fragment.

In one aspect, the antigen binding domains are scFv fragments.

In one aspect, the antigen binding domains are scFab fragments.

In one aspect, the antigen binding domains are fused to the C-terminus of the heavy chains of a) and/or b).

In one aspect, the trispecific or tetraspecific antibody comprises under c) one or two antigen binding domains which specifically bind to one further antigen.

In one aspect, the trispecific or tetraspecific antibody comprises under c) two identical antigen binding domains which specifically bind to a third antigen. In one preferred embodiment such two identical antigen binding domains are fused both via the same peptidic linker to the C-terminus of the heavy chains of a) and b). In one preferred embodiment the two identical antigen binding domains are either a scFv fragment or a scFab fragment.

In one aspect, the trispecific or tetraspecific antibody comprises under c) two antigen binding domains which specifically bind to a third and a fourth antigen. In one embodiment said two antigen binding domains are fused both via the same peptide connector to the C-terminus of the heavy chains of a) and b). In one preferred embodiment said two antigen binding domains are either a scFv fragment or a scFab fragment.

In one aspect, the bispecific antibody is a bispecific, tetravalent antibody comprising
a) two light chains and two heavy chains of an antibody, which specifically bind to a first antigen (and comprise two Fab fragments),
b) two additional Fab fragments of an antibody, which specifically bind to a second antigen, wherein said additional Fab fragments are fused both via a peptidic linker either to the C- or N-termini of the heavy chains of a), and
wherein in the Fab fragments the following modifications were performed
(i) in both Fab fragments of a), or in both Fab fragments of b), the variable domains VL and VH are replaced by each other, and/or the constant domains CL and CH1 are replaced by each other, or
(ii) in both Fab fragments of a) the variable domains VL and VH are replaced by each other, and the constant domains CL and CH1 are replaced by each other, and in both Fab fragments of b) the variable domains VL and VH are replaced by each other, or the constant domains CL and CH1 are replaced by each other, or
(iii) in both Fab fragments of a) the variable domains VL and VH are replaced by each other, or the constant domains CL and CH1 are replaced by each other, and in both Fab fragments of b) the variable domains VL and VH are replaced by each other, and the constant domains CL and CH1 are replaced by each other, or
(iv) in both Fab fragments of a) the variable domains VL and VH are replaced by each other, and in both Fab fragments of b) the constant domains CL and CH1 are replaced by each other, or
(v) in both Fab fragments of a) the constant domains CL and CH1 are replaced by each other, and in both Fab fragments of b) the variable domains VL and VH are replaced by each other.

In one aspect, said additional Fab fragments are fused both via a peptidic linker either to the C-termini of the heavy chains of a), or to the N-termini of the heavy chains of a).

In one aspect, said additional Fab fragments are fused both via a peptidic linker either to the C-termini of the heavy chains of a).

In one aspect, said additional Fab fragments are fused both via a peptide linker to the N-termini of the heavy chains of a).

In one aspect, in the Fab fragments the following modifications are performed: in both Fab fragments of a), or in both Fab fragments of b), the variable domains VL and VH are replaced by each other, and/or the constant domains CL and CH1 are replaced by each other.

In one aspect, the bispecific antibody is a tetravalent antibody comprising:
a) a (modified) heavy chain of a first antibody, which specifically binds to a first antigen and comprises a first VH—CH1 domain pair, wherein to the C terminus of said heavy chain the N-terminus of a second VH—CH1 domain pair of said first antibody is fused via a peptide linker,
b) two light chains of said first antibody of a),
c) a (modified) heavy chain of a second antibody, which specifically binds to a second antigen and comprises a first VH-CL domain pair, wherein to the C-terminus of said heavy chain the N-terminus of a second VH-CL domain pair of said second antibody is fused via a peptide linker, and
d) two (modified) light chains of said second antibody of c), each comprising a CL-CH1 domain pair.

In one aspect, the bispecific antibody comprises
a) the heavy chain and the light chain of a first full length antibody that specifically binds to a first antigen, and
b) the heavy chain and the light chain of a second full length antibody that specifically binds to a second antigen, wherein the N-terminus of the heavy chain is connected to the C-terminus of the light chain via a peptide linker.

The antibody under a) does not contain a modification as reported under b) and the heavy chain and the light chain are isolated chains.

In one aspect, the bispecific antibody comprises
a) a full length antibody specifically binding to a first antigen and consisting of two antibody heavy chains and two antibody light chains, and
b) an Fv fragment specifically binding to a second antigen comprising a VH2 domain and a VL2 domain, wherein both domains are connected to each other via a disulfide bridge, wherein only either the VH2 domain or the VL2 domain is fused via a peptide linker to the heavy or light chain of the full length antibody specifically binding to a first antigen.

In the bispecific antibody the heavy chains and the light chains under a) are isolated chains.

In one aspect, the other of the VH2 domain or the VL2 domain is not fused via a peptide linker to the heavy or light chain of the full length antibody specifically binding to a first antigen.

In all aspects as reported herein the first light chain comprises a VL domain and a CL domain and the first heavy chain comprises a VH domain, a CH1 domain, a hinge region, a CH2 domain and a CH3 domain.

In one aspect, the bispecific antibody is a trivalent antibody comprising
a) two Fab fragments that specifically binds to a first antigen,
b) one CrossFab fragment that specifically binds to a second antigen in which the CH1 and the CL domain are exchanged for each other,
c) one Fc-region comprising a first Fc-region heavy chain and a second Fc region heavy chain,
wherein the C-terminus of CH1 domains of the two Fab fragments are connected to the N-terminus of the heavy chain Fc-region polypeptides, and wherein the C-terminus of the CL domain of the CrossFab fragment is connected to the N-terminus of the VH domain of one of the Fab fragments.

In one aspect, the bispecific antibody is a trivalent antibody comprising
a) two Fab fragments that specifically binds to a first antigen,
b) one CrossFab fragment that specifically binds to a second antigen in which the CH1 and the CL domain are exchanged for each other,
c) one Fc-region comprising a first Fc-region heavy chain and a second Fc region heavy chain,
wherein the C-terminus of CH1 domain of the first Fab fragment is connected to the N-terminus of one of the heavy chain Fc-region polypeptides and the C-terminus of the CL-domain of the CrossFab fragment is connected to the N-terminus of the other heavy chain Fc-region polypeptide, and wherein the C-terminus of the CH1 domain of the second Fab fragment is connected to the N-terminus of the VH domain of the first Fab fragment or to the N-terminus of the VH domain of the CrossFab fragment.

In one aspect, the bispecific antibody comprises
a) a full length antibody specifically binding to a first antigen and consisting of two antibody heavy chains and two antibody light chains, and
b) a Fab fragment specifically binding to a second antigen comprising a VH2 domain and a VL2 domain comprising a heavy chain fragment and a light chain fragment, wherein within the light chain fragment the variable light chain domain VL2 is replaced by the variable heavy chain domain VH2 of said antibody, and within the heavy chain fragment the variable heavy chain domain VH2 is replaced by the variable light chain domain VL2 of said antibody
wherein the heavy chain Fab fragment is inserted between the CH1 domain of one of the heavy chains of the full length antibody and the respective Fc-region of the full length antibody, and the N-terminus of the light chain Fab fragment is conjugated to the C-terminus of the light chain of the full length antibody that is paired with the heavy chain of the full length antibody into which the heavy chain Fab fragment has been inserted.

In one aspect, the bispecific antibody comprises
a) a full length antibody specifically binding to a first antigen and consisting of two antibody heavy chains and two antibody light chains, and
b) a Fab fragment specifically binding to a second antigen comprising a VH2 domain and a VL2 domain comprising a heavy chain fragment and a light chain fragment, wherein within the light chain fragment the variable light chain domain VL2 is replaced by the variable heavy chain domain VH2 of said antibody, and within the heavy chain fragment the variable heavy chain domain VH2 is replaced by the variable light chain domain VL2 of said antibody and wherein the C-terminus of the heavy chain fragment of the Fab fragment is conjugated to the N-terminus of one of the heavy chains of the full length antibody and the C-terminus of the light chain fragment of the Fab fragment is conjugated to the N-terminus of the light chain of the full length antibody that pairs with the heavy chain of the full length antibody to which the heavy chain fragment of the Fab fragment is conjugated.

### Polynucleotides

Provided are furthermore isolated polynucleotides encoding a bispecific antibody as described herein or a fragment thereof.

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi: 10.1038/nm.4356 or EP 2 101 823 B1).

An “isolated” polynucleotide refers to a nucleic acid molecule that has been separated from a component of its natural environment. An isolated polynucleotide includes a nucleic acid molecule contained in cells that ordinarily contain the nucleic acid molecule, but the nucleic acid molecule is present extrachromosomally or at a chromosomal location that is different from its natural chromosomal location.

The isolated polynucleotides encoding bispecific antibodies of the invention may be expressed as a single polynucleotide that encodes the entire antigen binding molecule or as multiple (e.g., two or more) polynucleotides that are co-expressed. Polypeptides encoded by polynucleotides that are co-expressed may associate through, e.g., disulfide bonds or other means to form a functional antigen binding molecule. For example, the light chain portion of an immunoglobulin may be encoded by a separate polynucleotide from the heavy chain portion of the immunoglobulin. When co-expressed, the heavy chain polypeptides will associate with the light chain polypeptides to form the immunoglobulin.

In some aspects, the isolated polynucleotide encodes a polypeptide comprised in the bispecific antibody according to the invention as described herein.

In one aspect, the isolated polynucleotides are provided encoding an anti-PD1/anti-LAG3 bispecific antibody, wherein said first antigen binding domain specifically binding to PD1 comprises a VH domain comprising (i) CDR-H1 comprising the amino acid sequence of SEQ ID NO: 1, (ii) CDR-H2 comprising the amino acid sequence of SEQ ID NO:2, and (iii) CDR-H3 comprising an amino acid sequence of SEQ ID NO:3; and a VL domain comprising (i) CDR-L1 comprising the amino acid sequence of SEQ ID NO:4; (ii) CDR-L2 comprising the amino acid sequence of SEQ ID NO:5, and (iii) CDR-L3 comprising the amino acid sequence of SEQ ID NO: 6.

### Preparation of Bispecific Antibodies for Use in the Invention

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors. In case of certain bispecific antibodies with heterodimeric heavy chains four nucleic acids are required, one for the first light chain, one for the first heavy chain comprising the first hetreomonomeric Fc-region polypeptide, one for the second light chain, and one for the second heavy chain comprising the second heteromonomeric Fc-region polypeptide. The four nucleic acids can be comprised in one or more nucleic acid molecules or expression vectors. For example, such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. Examples of these bispecific antibodies are CrossMabs and T cell bispecifics (see, e.g. Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73).

In one aspect, isolated nucleic acid encoding a bispecific antibody described herein is provided. Such nucleic acid may encode an amino acid sequence comprising the VL and/or an amino acid sequence comprising the VH of the antigen binding domains that specifically bind to PD1 and LAG3, respectively (e.g., in the light and/or heavy chains of the antibody). In a further aspect, one or more vectors (e.g., expression vectors) comprising such nucleic acid are provided. In a further aspect, a host cell comprising such nucleic acid is provided. In one such aspect, a host cell comprises (e.g., has been transformed with): (1) a first vector comprising a first pair of nucleic acids that encode amino acid sequences one of them comprising the first VL and the other comprising the first VH of the antibody and a second vector comprising a second pair of nucleic acids that encode amino acid sequences one of them comprising the second VL and the other comprising the second VH of the antibody, or (2) a first vector comprising a first nucleic acid that encode an amino acid sequence comprising one of the variable domains (preferably a light chain variable domain), a second vector comprising a pair of nucleic acids that encode amino acid sequences one of them comprising a light chain variable domain and the other comprising the first heavy chain variable domain, and a third vector comprising a pair of nucleic acids that encode amino acid sequences one of them comprising the respective other light chain variable domain as in the second vector and the other comprising the second heavy chain variable domain, or (3) a first vector comprising a nucleic acid that encodes an amino acid sequence comprising the first VL of the antibody, a second vector comprising a nucleic acid that encodes an amino acid sequence comprising the first VH of the antibody, a third vector comprising a nucleic acid that encodes an amino acid sequence comprising the second VL of the antibody, and a fourth vector comprising a nucleic acid that encodes an amino acid sequence comprising the second VH of the antibody. In one aspect, the host cell is eukaryotic, e.g. a Chinese Hamster Ovary (CHO) cell or lymphoid cell (e.g., Y0, NS0, Sp20 cell). In one aspect, a method of making a bispecific antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of the anti-HLA-G/anti-CD3 bispecific antibodies and/or the anti-PD1/anti-LAG3 bispecific antibodies described herein, nucleic acid encoding the bispecific antibodies, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P. et al., Annals N.Y. Acad. Sci. 383 (1982) 44-68; MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

### Assays

The bispecific antibodies provided herein may be identified, screened for, or characterized for their physical/chemical properties and/or biological activities by various assays known in the art.

### 1. Affinity Assays

The affinity of the bispecific antigen binding molecules, antibodies and antibody fragments provided herein for the corresponding antigens can be determined in accordance with the methods set forth in the Examples by surface plasmon resonance (SPR), using standard instrumentation such as a Biacore® instrument (GE Healthcare), and receptors or target proteins such as may be obtained by recombinant expression. A specific illustrative and exemplary embodiment for measuring binding affinity has been described in Examples 2, 8 or 11 of WO 2018/185043. According to one aspect, K_{D} is measured by surface plasmon resonance using a BIACORE® T100 machine (GE Healthcare) at 25° C.

### 2. Binding Assays and Other Assays

In one aspect, the bispecific antibodies of the invention are tested for its antigen binding activity, e.g., by known methods such as ELISA, Western blot, etc. The binding of the anti-PD1/anti-LAG3 bispecific antibodies provided herein to the corresponding recombinant antigen or to antigen-expressing cells may be evaluated by ELISA as described in Examples 8 or 11 of WO 2018/185043. In a further aspect, fresh peripheral blood mononuclear cells (PBMCs) can be used in binding assays to show binding to different peripheral blood mononuclear cells (PBMC) such as monocytes, NK cells and T cells.

In another aspect, a cellular dimerization assay was used to demonstrate the dimerization or at least binding/interaction of two different receptors PD1 and LAG3, which are cytosolically fused with two fragments of an enzyme, upon ligation or cross-linking with a bispecific antibody against both targets. Hereby only one receptor alone shows no enzymatic activity. For this specific interaction, the cytosolic C-terminal ends of both receptors were individually fused to heterologous subunits of a reporter enzym. A single enzyme subunit alone showed no reporter activity.

However, simultaneous binding to both receptors was expected to lead to local cytocolic accumulation of both receptors, complementation of the two heterologous enzyme subunits, and finally to result in the formation of a specific and functional enzyme that hydrolyzes a substrate thereby generating a chemiluminescent signal (Example 11 of WO 2018/185043).

### 3. Activity Assays

In one aspect, assays are provided for identifying an anti-PD1/anti-LAG3 bispecific antibody having biological activity. Biological activity may include, e.g., the ability to enhance the activation and/or proliferation of different immune cells, especially T cells, secretion of immune-modulating cytokines such IFNγ or TNF-alpha, blocking the PD1 pathway, blocking the LAG3 pathway, killing of tumor cells. Antibodies having such biological activity in vivo and/or in vitro are also provided. In certain aspects, an antibody of the invention is tested for such biological activity. In one aspect, provided is an immune cell assay which measures the activation of lymphocytes from one individual (donor X) to lymphocytes from another individual (donor Y). The mixed lymphocyte reaction (MLR) can demonstrate the effect of blocking the PD1 pathway to lymphocyte effector cells. T cells in the assay were tested for activation and their IFN-gamma secretion in the presence or absence of bispecific antibodies of the invention. The assay is described in more detail in Example 9 of WO 2018/185043.

### Pharmaceutical Compositions, Formulations and Routes of Administration

In a further aspect, the invention provides pharmaceutical compositions comprising the anti-HLA-G/anti-CD3 antibodies and anti-PD1/anti-LAG3 antibodies provided herein, e.g., for use in any of the below therapeutic methods. In one embodiment, a pharmaceutical composition comprises an anti-HLA-G/anti-CD3 antibody and an anti-PD1/anti-LAG3 antibody provided herein and at least one pharmaceutically acceptable excipient. In another embodiment, a pharmaceutical composition comprises an antibody provided herein and at least one additional therapeutic agent, e.g., as described below.

Pharmaceutical compositions of the present invention comprise a therapeutically effective amount of one or more bispecific antibodies dissolved or dispersed in a pharmaceutically acceptable excipient. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one antibody and optionally an additional active ingredient will be known to those of skill in the art in light of the present disclosure, as exemplified by Remington's Pharmaceutical Sciences, 18th Ed. Mack Printing Company, 1990, incorporated herein by reference. In particular, the compositions are lyophilized formulations or aqueous solutions. As used herein, “pharmaceutically acceptable excipient” includes any and all solvents, buffers, dispersion media, coatings, surfactants, antioxidants, preservatives (e.g. antibacterial agents, antifungal agents), isotonic agents, salts, stabilizers and combinations thereof, as would be known to one of ordinary skill in the art.

Parenteral compositions include those designed for administration by injection, e.g. subcutaneous, intradermal, intralesional, intravenous, intraarterial intramuscular, intrathecal or intraperitoneal injection. For injection, the antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the fusion proteins may be in powder form for constitution with a suitable vehicle, e.g., sterile pyrogen-free water, before use. Sterile injectable solutions are prepared by incorporating the fusion proteins of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable excipients include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol: alkyl parabens such as methyl or propyl paraben; catechol; resorcinol; cyclohexanol: 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

Exemplary pharmaceutically acceptable excipients herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

In addition to the compositions described previously, the bispecific antibodies may also be formulated as a depot preparation. Such long acting formulations may be administered by implantation (for example subcutaneously or intramuscularly) or by intramuscular injection. Thus, for example, the fusion proteins may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the bispecific antigen binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The bispecific antibodies disclosed herein may be formulated into a composition in a free acid or base, neutral or salt form. Pharmaceutically acceptable salts are salts that substantially retain the biological activity of the free acid or base. These include the acid addition salts, e.g. those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

The composition herein may also contain more than one active ingredients as necessary for the particular indication being treated, preferably those with complementary activities that do not adversely affect each other. Such active ingredients are suitably present in combination in amounts that are effective for the purpose intended.

In one aspect, there is provided a pharmaceutical composition comprising an anti-HLA-G/anti-CD3 bispecific antibody and a pharmaceutically acceptable carrier, and a second medicament comprising an anti-PD1/anti-LAG3 antibody as described herein. In one aspect, the pharmaceutical composition is for use in the treatment of an HLA-G-expressing cancer. In a particular aspect, the pharmaceutical composition is for use in the treatment of HLA-G expressing cancer, in particular a disease selected from lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, carcinoma of the fallopian tubes, carcinoma of the endometrium, carcinoma of the cervix, carcinoma of the vagina, carcinoma of the vulva, Hodgkin's Disease, nasopharyngeal carcinoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymomas, medulloblastomas, meningiomas, squamous cell carcinomas, pituitary adenoma, lymphoma, lymphocytic leukemia, including refractory versions of any of the above cancers, or a combination of one or more of the above cancers.

The formulations to be used for in vivo administration are generally sterile. Sterility may be readily accomplished, e.g., by filtration through sterile filtration membranes.

### Administration of the Anti-HLA-G/Anti-CD3 Bispecific Antibody and the Anti-PD1/Anti-LAG3 Antibody

Both the anti-HLA-G/anti-CD3 bispecific antibody and the anti-PD1/anti-LAG3 antibody (both called substance herein) can be administered by any suitable means, including parenteral, intrapulmonary, and intranasal, and, if desired for local treatment, intralesional administration. The methods described herein are particularly useful, however, in relation to therapeutic agents administered by parenteral, particularly intravenous, infusion.

Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein. In one aspect, the therapeutic agent is administered parenterally, particularly intravenously. In a particular aspect, the substance is administered by intravenous infusion. In another aspect, the substance is administered subcutaneously.

Both the anti-HLA-G/anti-CD3 bispecific antibody and the anti-PD1/anti-LAG3 antibody would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. Both the anti-HLA-G/anti-CD3 bispecific antibody and the anti-PD1/anti-LAG3 antibody need not be, but are optionally formulated with one or more agents currently used to prevent or treat the disorder in question. The effective amount of such other agents depends on the amount of therapeutic agent present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of the anti-HLA-G/anti-CD3 bispecific antibody and the anti-PD1/anti-LAG3 antibody (when used in their combination or with one or more other additional therapeutic agents) will depend on the type of disease to be treated, the type of anti-HLA-G/anti-CD3 bispecific antibody, the severity and course of the disease, whether both agents are administered for preventive or therapeutic purposes, previous therapy, the patient's clinical history and response to the therapeutic agent, and the discretion of the attending physician. Each substance is suitably administered to the patient at one time or over a series of treatments. Depending on the type and severity of the disease, about 1 μg/kg to 15 mg/kg (e.g. 0.1 mg/kg-10 mg/kg) of the substance can be an initial candidate dosage for administration to the subject, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the bispecific antibody would be in the range from about 0.005 mg/kg to about 10 mg/kg. In other examples, a dose may also comprise from about 1 μg/kg body weight, about 5 μg/kg body weight, about 10 μg/kg body weight, about 50 μg/kg body weight, about 100 μg/kg body weight, about 200 μg/kg body weight, about 350 μg/kg body weight, about 500 μg/kg body weight, about 1 mg/kg body weight, about 5 mg/kg body weight, about 10 mg/kg body weight, about 50 mg/kg body weight, about 100 mg/kg body weight, about 200 mg/kg body weight, about 350 mg/kg body weight, about 500 mg/kg body weight, to about 1000 mg/kg body weight or more per administration, and any range derivable therein. In examples of a derivable range from the numbers listed herein, a range of about 5 mg/kg body weight to about 100 mg/kg body weight, about 5 μg/kg body weight to about 500 mg/kg body weight etc., can be administered, based on the numbers described above. Thus, one or more doses of about 0.5 mg/kg. 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays. However, other dosage regimens may be useful. The progress of this therapy is easily monitored by conventional techniques and assays.

In one aspect, the administration of both the anti-HLA-G/anti-CD3 bispecific antibody and the anti-PD1/anti-LAG3 antibody is a single administration. In certain aspects, the administration of the therapeutic agent is two or more administrations. In one such aspect, the substances are administered every week, every two weeks, or every three weeks, particularly every two weeks. In one aspect, the substance is administered in a therapeutically effective amount. In one aspect the substance is administered at a dose of about 10 μg/kg, about 100 μg/kg, about 200 μg/kg, about 300 μg/kg, about 400 μg/kg, about 500 μg/kg, about 600 μg/kg, about 700 μg/kg, about 800 μg/kg, about 900 μg/kg or about 1000 μg/kg. In one embodiment, the anti-HLA-G/anti-CD3 bispecific antibody is administered at a dose which is higher than the dose of the anti-HLA-G/anti-CD3 bispecific antibody in a corresponding treatment regimen without the administration of the anti-PD1/anti-LAG3 antibody. In one aspect, the administration of the anti-HLA-G/anti-CD3 bispecific antibody comprises an initial administration of a first dose of the anti-HLA-G/anti-CD3 bispecific antibody, and one or more subsequent administrations of a second dose of the anti-HLA-G/anti-CD3 bispecific antibody, wherein the second dose is higher than the first dose. In one aspect, the administration of the anti-HLA-G/anti-CD3 bispecific antibody comprises an initial administration of a first dose of the anti-HLA-G/anti-CD3 bispecific antibody, and one or more subsequent administrations of a second dose of the anti-HLA-G/anti-CD3 bispecific antibody, wherein the first dose is not lower than the second dose.

In one aspect, the administration of the anti-HLA-G/anti-CD3 bispecific antibody in the treatment regimen according to the invention is the first administration of the anti-HLA-G/anti-CD3 bispecific antibody to the subject (at least within the same course of treatment). In one aspect, no administration of the anti-PD1/anti-LAG3 antibody is made to the subject prior to the administration of the anti-HLA-G/anti-CD3 bispecific antibody. In another aspect, the anti-PD1/anti-LAG3 antibody is administered prior to the administration of the anti-HLA-G/anti-CD3 bispecific antibody.

In the present invention, the combination of the anti-HLA-G/anti-CD3 bispecific antibody and the anti-PD1/anti-LAG3 antibody can be used in combination with one or more further agents in a therapy. For instance, at least one additional therapeutic agent may be co-administered. In certain aspects, an additional therapeutic agent is an immunotherapeutic agent.

Such combination therapies noted above encompass combined administration (where two or more therapeutic agents are included in the same or separate formulations), and separate administration, in which case, administration of the therapeutic agent can occur prior to, simultaneously, and/or following, administration of an additional therapeutic agent or agents. In one embodiment, administration of the therapeutic agent and administration of an additional therapeutic agent occur within about one month, or within about one, two or three weeks, or within about one, two, three, four, five, or six days, of each other.

### Therapeutic Methods and Compositions

HLA-G is predominantly expressed on cytotrophoblasts in the placenta, but also various tumors (including pancreatic, breast, skin, colorectal, gastric & ovarian) have been reported to express HLA-G.

In one aspect, there is provided a method for treating or delaying progression of HLA-G-expressing cancer in a subject comprising administering to the subject an effective amount of an anti-HLA-G/anti-CD3 antibody and an effective amount of an anti-PD1/anti-LAG3 antibody.

In one such aspect, the method further comprises administering to the subject an effective amount of at least one additional therapeutic agent. In some embodiments, herein is provided a method for increasing cytokine secretion (including IFNγ) and/or T cell activation comprising administering to the subject an effective amount of an anti-HLA-G/anti-CD3 antibody and an effective amount of an anti-PD1/anti-LAG3 antibody. In particular embodiments, the method comprises T cell mediated killing of tumor cells. An “individual” or a “subject” according to any of the above aspects is preferably a human.

In further aspects, a composition for use in cancer immunotherapy is provided comprising an anti-HLA-G/anti-CD3 antibody and an anti-PD1/anti-LAG3 antibody. In certain embodiments, a composition comprising an anti-HLA-G/anti-CD3 antibody and an effective amount of an anti-PD1/anti-LAG3 antibody for use in a method of cancer immunotherapy is provided.

In a further aspect, herein is provided the use of a composition comprising an anti-HLA-G/anti-CD3 antibody and an effective amount of an anti-PD1/anti-LAG3 antibody in the manufacture or preparation of a medicament. In one aspect, the medicament is for treatment of an HLA-G-expressing cancer. In one aspect, the medicament is for treatment of an HLA-G expressing cancer. In a further aspect, the medicament is for use in a method of treating an HLA-G expressing cancer comprising administering to an individual having an HLA-G expressing cancer an effective amount of the medicament. In one such aspect, the method further comprises administering to the individual an effective amount of at least one additional therapeutic agent. In a further aspect, the medicament is for activating T cells, in particular against HLA-G expressing tumor cells. HLA-G expressing cancers are selected from lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, carcinoma of the fallopian tubes, carcinoma of the endometrium, carcinoma of the cervix, carcinoma of the vagina, carcinoma of the vulva, Hodgkin's Disease, nasopharyngeal carcinoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymomas, medulloblastomas, meningiomas, squamous cell carcinomas, pituitary adenoma, lymphoma, lymphocytic leukemia, including refractory versions of any of the above cancers, or a combination of one or more of the above cancers. In particular, HLA-G expressing cancers are selected renal cell carcinoma, colorectal carcinoma, non-small cell lung cancer and pancreatic duct adenocarcinoma (PDAC).

In one such embodiment, the method further comprises administering to the individual an effective amount of at least one additional therapeutic agent, as described below. An “individual” according to any of the above embodiments may be a human.

The combination therapies noted above encompass combined administration (where two or more therapeutic agents are included in the same or separate formulations), and separate administration, in which case, administration of the anti-PD1/anti-LAG3 bispecific antibody as reported herein can occur prior to, simultaneously, and/or following, administration of the additional therapeutic agent or agents. In one aspect, administration of the effective amount of an anti-HLA-G/anti-CD3 bispecific antibody, of the effective amount of an anti-PD1/anti-LAG3 antibody and administration of an additional therapeutic agent occur within about one month, or within about one, two or three weeks, or within about one, two, three, four, five, or six days, of each other.

Both the anti-HLA-G/anti-CD3 bispecific antibody and the anti-PD1/anti-LAG3 antibody as reported herein (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Both the anti-HLA-G/anti-CD3 bispecific antibody and the anti-PD1/anti-LAG3 antibody as reported herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibodies need not be, but are optionally formulated with one or more agents currently used to prevent or treat the disorder in question. The effective amount of such other agents depends on the amount of antibodies present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

A skilled artisan readily recognizes that in many cases the bispecific molecule may not provide a cure but may only provide partial benefit. In some embodiments, a physiological change having some benefit is also considered therapeutically beneficial. Thus, in some aspects, an amount of the bispecific antibody that provides a physiological change is considered an “effective amount” or a “therapeutically effective amount”.

Both the anti-HLA-G/anti-CD3 bispecific antibody and the anti-PD1/anti-LAG3 antibody as defined herein is suitably administered to the patient at one time or over a series of treatments. Depending on the type and severity of the disease, about 1 μg/kg to 15 mg/kg (e.g. 0.1 mg/kg-10 mg/kg) of the bispecific antibody can be an initial candidate dosage for administration to the patient, whether, for example, by one or more separate administrations, or by continuous infusion. One typical daily dosage might range from about 1 μg/kg to 10 mg/kg or more, depending on the factors mentioned above. For repeated administrations over several days or longer, depending on the condition, the treatment would generally be sustained until a desired suppression of disease symptoms occurs. One exemplary dosage of the anti-HLA-G/anti-CD3 bispecific antibody would be in the range from about 0.05 μg/kg to about 1000 μg/kg. For the anti-PD1/anti-LAG3 antibody, a dose may also comprise from about 0.01 mg/kg body weight, about 0.05 mg/kg body weight, about 2 mg/kg body weight, about 4 mg/kg body weight, about 10 mg/kg body weight, about 20 mg/kg body weight, about 30 mg/kg body weight, about 40 mg/kg body weight, about 45 mg/kg body weight, about 50 mg/kg body weight, about 100 mg/kg body weight, about 200 mg/kg body weight, about 300 mg/kg body weight, about 400 mg/kg body weight, about 500 mg/kg body weight, about 600 mg/kg body weight, about 800 mg/kg body weight, about 1000 mg/kg body weight, top about 1200 mg/kg body weight or more per administration, and any range derivable therein. In examples of a derivable range from the numbers listed herein, a range of about 5 mg/kg body weight to about 100 mg/kg body weight, about 0.05 μg/kg body weight to about 500 mg/kg body weight etc., can be administered, based on the numbers described above. In one aspect, the anti-HLA-G/anti-CD3 bispecific antibody may be administered to the patient in a dose of from about 0.01 mg, from 2.5 mg, to about 10 mg or about 20 mg or about 30 mg. Such doses may be administered intermittently, e.g. every week or every three weeks (e.g. such that the patient receives from about two to about twenty, or e.g. about six doses of the fusion protein). An initial lower loading dose, followed by one or more higher doses may be administered. However, other dosage regimens may be useful. The progress of this therapy is easily monitored by conventional techniques and assays. In one aspect, the anti-PD1/anti-LAG3 antibody may be administered to the patient in a dose of from about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg, about 1400 mg or about 1500 mg.

The bispecific antibodies comprising a first antigen binding domain that specifically binds to PD1 and a second antigen binding domain that specifically binds to LAG3 as defined herein will generally be used in an amount effective to achieve the intended purpose. For use to treat or prevent a disease condition, the bispecific antibodies of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the ICso as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the bispecific antibody which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the bispecific antibody may not be related to plasma concentration. One skilled in the art will be able to optimize therapeutically effective local dosages without undue experimentation.

A therapeutically effective dose of the bispecific antibodies described herein will generally provide therapeutic benefit without causing substantial toxicity. Toxicity and therapeutic efficacy of a fusion protein can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Bispecific antibodies that exhibit large therapeutic indices are preferred. In one embodiment, the bispecific antibody according to the present invention exhibits a high therapeutic index. The data obtained from cell culture assays and animal studies can be used in formulating a range of dosages suitable for use in humans. The dosage lies preferably within a range of circulating concentrations that include the ED50 with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with bispecific antibodies of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

Such other agents are suitably present in combination in amounts that are effective for the purpose intended. The effective amount of such other agents depends on the amount of fusion protein used, the type of disorder or treatment, and other factors discussed above. The bispecific antibodies are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the bispecific antibody can occur prior to, simultaneously, and/or following, administration of the additional therapeutic agent and/or adjuvant.

### H. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper that is pierceable by a hypodermic injection needle). At least one active agent in the composition is an anti-PD1/anti-LAG3 antibody as defined herein before.

The label or package insert indicates that the composition is used for treating the condition of choice. Moreover, the article of manufacture may comprise (a) a first container with a composition contained therein, wherein the composition comprises an anti-HLA-G/anti-CD3 bispecific antibody and (b) a second container with a composition contained therein, wherein the composition comprises an anti-PD1/anti-LAG3 antibody. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

TABLE B
(Sequences):

| SEQ | | |
|---|---|---|
| ID | | |
| NO: | Name | Sequence |
| 1 | heavy chain CDR-H1, PD1- | SYTMS |
| | 0103 | |
| 2 | heavy chain CDR-H2, PD1- | TISGGGRDIY YPDSVKG |
| | 0103 | |
| 3 | heavy chain CDR-H3, PD1- | LTGRVYFALD S |
| | 0103 | |
| 4 | light chain CDR-L1, PD1- | KASESVDTSD NSFIH |
| | 0103 | |
| 5 | light chain CDR-L2, PD1- | RSSTLES |
| | 0103 | |
| 6 | light chain CDR-L3, PD1- | QQNYDVPWT |
| | 0103 | |
| 7 | heavy chain variable | EVILVESGGGLVKPGGSLKLSCAASGFSFSSYTM |
| | domain VH, PD1-0103 | SWVRQTPEKRLDWVATISGGGRDIYYPDSVKGRF |
| | | TISRDNAKNTLYLEMSSLMSEDTALYYCVLLTGR |
| | | VYFALDSWGQGTSVTVSS |
| 8 | light chain variable domain | KIVLTQSPASLPVSLGQRATISCRASESVDTSDN |
| | VL, PD1-0103 | SFIHWYQQRPGQSPKLLIYRSSTLESGVPARFSG |
| | | SGSRTDFTLTIDPVEADDVATYYCQQNYDVPWTF |
| | | GGGTKLEIK |
| 9 | heavy chain variable | EVQLLESGGGLVQPGGSLRLSCAASGFSFSSYTM |
| | domain VH (humanized | SWVRQAPGKGLEWVATISGGGRDIYYPDSVKGRF |
| | variant), PD1-0103_01 | TISRDNSKNTLYLQMNSLRAEDTAVYYCVLLTGR |
| | (PD1 0376) | VYFALDSWGQGTLVTVSS |
| 10 | light chain variable domain | DIVMTQSPDSLAVSLGERATINCKASESVDTSDN |
| | VL (humanized variant), | SFIHWYQQKPGQSPKLLIYRSSTLESGVPDRFSG |
| | PD1-0103_01 (PD1 0376) | SGSGTDFTLTISSLQAEDVAVYYCQQNYDVPWTF |
| | | GQGTKVEIK |
| 11 | heavy chain CDR-H1, | DYTMN |
| | aLAG3(0414) | |
| 12 | heavy chain CDR-H2, | VISWDGGGTY YTDSVKG |
| | aLAG3(0414) | |
| 13 | heavy chain CDR-H3, | GLTDTTLYGS DY |
| | aLAG3(0414) | |
| 14 | light chain CDR-L1, | RASQSISSYL N |
| | aLAG3(0414) | |
| 15 | light chain CDR-L2, | AASTLQS |
| | aLAG3(0414) | |
| 16 | light chain CDR-L3, | QQTYSSPLT |
| | aLAG3(0414) | |
| 17 | heavy chain variable | EVQLLESGGGLVQPGGSLRLSCAASGFIFDDYTM |
| | domain VH, aLAG3(0414) | NWVRQAPGKGLEWVAVISWDGGGTYYTDSVKGRF |
| | | TISRDDFKNTLYLQMNSLRAEDTAVYYCAKGLTD |
| | | TTLYGSDYWGQGTLVTVSS |
| 18 | light chain variable domain | DIQMTQSPSSLSASVGDRVTITCRASQSISSYLN |
| | VL, aLAG3(0414) | WYQQKPGKAPKLLIYAASTLQSGVPSRFSGSGSG |
| | | TDFTLTISSLQPEDFATYYCQQTYSSPLTFGGGT |
| | | KVEIK |
| 19 | heavy chain CDR-H1, | DYAMS |
| | aLAG3(0416) | |
| 20 | heavy chain CDR-H2, | GIDNSGYYTY YTDSVKG |
| | aLAG3(0416) | |
| 21 | heavy chain CDR-H3, | THSGLIVNDA FDI |
| | aLAG3(0416) | |
| 22 | light chain CDR-L1, | RASQSISSYL N |
| | aLAG3(0416) | |
| 23 | light chain CDR-L2, | DASSLES |
| | aLAG3(0416) | |
| 24 | light chain CDR-L3, | QQSYSTPLT |
| | aLAG3(0416) | |
| 25 | heavy chain variable | EVQLVESGGGLVQPGGSLRLACAASGFTFSDYAM |
| | domain VH, aLAG3(0416) | SWVRQAPGKGLEWVSGIDNSGYYTYYTDSVKGRF |
| | | TISRDDVKNTLYLQMNSLRAEDTAVYLCTKTHSG |
| | | LIVNDAFDIWGQGTMVTVSS |
| 26 | light chain variable domain | DIQLTQSPSSLSASVGDRVTITCRASQSISSYLN |
| | VL, aLAG3(0416) | WYQQKPGKAPKLLIYDASSLESGVPSRFSGSGSG |
| | | TDATLTISSLQPEDFATYYCQQSYSTPLTFGGGT |
| | | KVEIK |
| 27 | heavy chain variable | QVQLQQWGAGLLKPSETLSLTCAVYGGSFSDYYW |
| | domain VH, BMS-986016 | NWIRQPPGKGLEWIGEINHRGSTNSNPSLKSRVT |
| | | LSLDTSKNQFSLKLRSVTAADTAVYYCAFGYSDY |
| | | EYNWFDPWGQGTLVTVSS |
| 28 | light chain variable domain | EIVLTQSPATLSLSPGERATLSCRASQSISSYLA |
| | VL BMS-986016 | WYQQKPGQAPRLLIYDASNRATGIPARFSGSGSG |
| | | TDFTLTISSLEPEDFAVYYCQQRSNWPLTFGQGT |
| | | NLEIK |
| 29 | heavy chain variable | QVQLQQWGAGLLKPSETLSLTCAVYGGSFSDYYW |
| | domain VH, MDX25F7 | NWIRQPPGKGLEWIGEINHNGNTNSNPSLKSRVT |
| | (25F7) | LSLDTSKNQFSLKLRSVTAADTAVYYCAFGYSDY |
| | | EYNWFDPWGQGTLVTVSS |
| 30 | light chain variable domain | EIVLTQSPATLSLSPGERATLSCRASQSISSYLA |
| | VL, MDX25F7 (25F7) | WYQQKPGQAPRLLIYDASNRATGIPARFSGSGSG |
| | | TDFTLTISSLEPEDFAVYYCQQRSNWPLTFGQGT |
| | | NLEIK |
| 31 | heavy chain variable | QVQLVQSGAEVKKPGASVKVSCKASGFTLTNYGM |
| | domain VH, humanized | NWVRQARGQRLEWIGWINTDTGEPTYADDFKGRF |
| | BAP050 (LAG525) | VFSLDTSVSTAYLQISSLKAEDTAVYYCARNPPY |
| | | YYGTNNAEAMDYWGQGTTVTVSS |
| 32 | light chain variable domain | DIQMTQSPSSLSASVGDRVTITCSSSQDISNYLN |
| | VL, humanized BAP050 | WYLQKPGQSPQLLIYYTSTLHLGVPSRFSGSGSG |
| | (LAG525) | TEFTLTISSLQPDDFATYYCQQYYNLPWTFGQGT |
| | | KVEIK |
| 33 | heavy chain variable | QVQLVESGGGVVQPGRSLRLSCAASGFTFSSYGM |
| | domain VH, MDX26H10 | HWVRQAPGKGLEWVAVIWYDGSNKYYADSVKGRF |
| | (26H10) | TISRDNSKNTLYLQMNSLRAEDTAVYYCAREWAV |
| | | ASWDYGMDVWGQGTTVTVSS |
| 34 | light chain variable domain | EIVLTQSPGTLSLSPGERATLSCRASQSVSSSYL |
| | VL, MDX26H10 (26H10) | AWYQQKPGQAPRLLIYGASSRATGIPDRFSGSGS |
| | | GTDFTLTISRLEPEDFAVYYCQQYGSSPFTFG |
| | | PGTKVDIK |
| 35 | heavy chain 1 of 1 + 1 | DIVMTQSPDSLAVSLGERATINCKASESVDTSDN |
| | PD1/LAG3 0927 based on | SFIHWYQQKPGQSPKLLIYRSSTLESGVPDRFSG |
| | PD1(0376) | SGSGTDFTLTISSLQAEDVAVYYCQQNYDVPWTF |
| | | GQGTKVEIKSSASTKGPSVFPLAPSSKSTSGGTA |
| | | ALGCLVKDYFPEPVTVSWNSGALTSGVHTFPAVL |
| | | QSSGLYSLSSVVTVPSSSLGTQTYICNVNHKPSN |
| | | TKVDKKVEPKSCDKTHTCPPCPAPEAAGGPSVFL |
| | | FPPKPKDTLMISRTPEVTCVVVDVSHEDPEVKFN |
| | | WYVDGVEVHNAKTKPREEQYNSTYRVVSVLTVLH |
| | | QDWLNGKEYKCKVSNKALGAPIEKTISKAKGQPR |
| | | EPQVYTLPPCRDELTKNQVSLWCLVKGFYPSDIA |
| | | VEWESNGQPENNYKTTPPVLDSDGSFFLYSKLTV |
| | | DKSRWQQGNVFSCSVMHEALHNHYTQKSLSLSP |
| 36 | light chain 1 of 1 + 1 | EVQLLESGGGLVQPGGSLRLSCAASGFSFSSYTM |
| | PD1/LAG3 0927 based on | SWVRQAPGKGLEWVATISGGGRDIYYPDSVKGRF |
| | PD1(0376) | TISRDNSKNTLYLQMNSLRAEDTAVYYCVLLTGR |
| | | VYFALDSWGQGTLVTVSSASVAAPSVFIFPPSDE |
| | | QLKSGTASVVCLLNNFYPREAKVQWKVDNALQSG |
| | | NSQESVTEQDSKDSTYSLSSTLTLSKADYEKHKV |
| | | YACEVTHQGLSSPVTKSFNRGEC |
| 37 | heavy chain 2 of 1 + 1 | EVQLLESGGGLVQPGGSLRLSCAASGFIFDDYTM |
| | PD1/LAG3 0927 based on | NWVRQAPGKGLEWVAVISWDGGGTYYTDSVKGRF |
| | aLAG3(0414) | TISRDDFKNTLYLQMNSLRAEDTAVYYCAKGLTD |
| | | TTLYGSDYWGQGTLVTVSSASTKGPSVFPLAPSS |
| | | KSTSGGTAALGCLVEDYFPEPVTVSWNSGALTSG |
| | | VHTFPAVLQSSGLYSLSSVVTVPSSSLGTQTYIC |
| | | NVNHKPSNTKVDEKVEPKSCDKTHTCPPCPAPEA |
| | | AGGPSVFLFPPKPKDTLMISRTPEVTCVVVDVSH |
| | | EDPEVKFNWYVDGVEVHNAKTKPREEQYNSTYRV |
| | | VSVLTVLHQDWLNGKEYKCKVSNKALGAPIEKTI |
| | | SKAKGQPREPQVCTLPPSRDELTKNQVSLSCAVK |
| | | GFYPSDIAVEWESNGQPENNYKTTPPVLDSDGSF |
| | | FLVSKLTVDKSRWQQGNVFSCSVMHEALHNHYTQ |
| | | KSLSLSP |
| 38 | light chain 2 of 1 + 1 | DIQMTQSPSSLSASVGDRVTITCRASQSISSYLN |
| | PD1/LAG3 0927 based on | WYQQKPGKAPKLLIYAASTLQSGVPSRFSGSGSG |
| | aLAG3(0414) | TDFTLTISSLQPEDFATYYCQQTYSSPLTFGGGT |
| | | KVEIKRTVAAPSVFIFPPSDRKLKSGTASVVCLL |
| | | NNFYPREAKVQWKVDNALQSGNSQESVTEQDSKD |
| | | STYSLSSTLTLSKADYEKHKVYACEVTHQGLSSP |
| | | VTKSFNRGEC |
| 39 | heavy chain 2 of 1 + 1 | EVQLVESGGGLVQPGGSLRLACAASGFTFSDYAM |
| | PD1/LAG3 0799 based on | SWVRQAPGKGLEWVSGIDNSGYYTYYTDSVKGRF |
| | aLAG3(0416) | TISRDDVKNTLYLQMNSLRAEDTAVYLCTKTHSG |
| | | LIVNDAFDIWGQGTMVTVSSASTKGPSVFPLAPS |
| | | SKSTSGGTAALGCLVEDYFPEPVTVSWNSGALTS |
| | | GVHTFPAVLQSSGLYSLSSVVTVPSSSLGTQTYI |
| | | CNVNHKPSNTKVDEKVEPKSCDKTHTCPPCPAPE |
| | | AAGGPSVFLFPPKPKDTLMISRTPEVTCVVVDVS |
| | | HEDPEVKFNWYVDGVEVHNAKTKPREEQYNSTYR |
| | | VVSVLTVLHQDWLNGKEYKCKVSNKALGAPIEKT |
| | | ISKAKGQPREPQVCTLPPSRDELTKNQVSLSCAV |
| | | KGFYPSDIAVEWESNGQPENNYKTTPPVLDSDGS |
| | | FFLVSKLTVDKSRWQQGNVFSCSVMHEALHNHYT |
| | | QKSLSLSP |
| 40 | light chain 2 of 1 + 1 | DIQLTQSPSSLSASVGDRVTITCRASQSISSYLN |
| | PD1/LAG3 0799 based on | WYQQKPGKAPKLLIYDASSLESGVPSRFSGSGSG |
| | aLAG3(0416) | TDATLTISSLQPEDFATYYCQQSYSTPLTFGGGT |
| | | KVEIKRTVAAPSVFIFPPSDRKLKSGTASVVCLL |
| | | NNFYPREAKVQWKVDNALQSGNSQESVTEQDSKD |
| | | STYSLSSTLTLSKADYEKHKVYACEVTHQGLSSP |
| | | VTKSFNRGEC |
| 41 | heavy chain CDR-H1, CD3 | SYAMN |
| 42 | heavy chain CDR-H2, CD3 | RIRSKYNNYATYYADSVKG |
| 43 | heavy chain CDR-H3, CD3 | ASNFPASYVSYFAY |
| 44 | light chain CDR-L1, CD3 | GSSTGAVTTSNYAN |
| 45 | light chain CDR-L2, CD3 | GTNKRAP |
| 46 | light chain CDR-L3, CD3 | ALWYSNLWV |
| 47 | heavy chain variable | EVQLLESGGGLVQPGGSLRLSCAASGFTFSSYAM |
| | domain VH, CD3 | NWVRQAPGKGLEWVSRIRSKYNNYATYYADSVKG |
| | | RFTISRDDSKNTLYLQMNSLRAEDTAVYYCVRAS |
| | | NFPASYVSYFAYWGQGTLVTVSS |
| 48 | light chain variable domain | QAVVTQEPSLTVSPGGTVTLTCGSSTGAVTTSNY |
| | VL, CD3 | ANWVQEKPGQAFRGLIGGTNKRAPGTPARFSGSL |
| | | LGGKAALTLSGAQPEDEAEYYCALWYSNLWVFGG |
| | | GTKLTVL |
| 49 | heavy chain CDR-H1, | SNRAAWN |
| | HLA-G-0090-S32P | |
| 50 | heavy chain CDR-H2, | RTYYRSKWYNDYAVSVQG |
| | HLA-G-0090-S32P | |
| 51 | heavy chain CDR-H3, | VRAVAPFDY |
| | HLA-G-0090-S32P | |
| 52 | light chain CDR-L1, HLA- | KSSQSVLNP SNNKNNLA |
| | G-0090-S32P | |
| 53 | light chain CDR-L2, HLA- | WASTRES |
| | G-0090-S32P | |
| 54 | light chain CDR-L3, HLA- | QQYYRTPWT |
| | G-0090-S32P | |
| 55 | heavy chain variable | QVQLQQSGPGLLKPSQTLSLTCAISGDSVSSNRA |
| | domain VH, HLA-G-0090- | AWNWIRQSPSRGLEWLGRTYYRSKWYNDYAVSVQ |
| | S32P | GRITLIPDTSKNQFSLRLNSVTPEDTAVYYCASV |
| | | RAVAPFDYWGQGVLVTVSS |
| 56 | light chain variable domain | DIVMTQSPDSLAVSLGERATINCKSSQSVLNPSN |
| | VL, HLA-G-0090-S32P | NKNNLAWYQQQPGQPPKLLIYWASTRESGVPDRF |
| | | SGSGSGTDFTLTISSLQAEDVAVYFCQQYYRTPW |
| | | TFGQGTKVEIK |
| 57 | HLA-G VH-CH1(EE)-CD3 | QVQLQQSGPGLLKPSQTLSLTCAISGDSVSSNRA |
| | VL-CH1-Fc (knob, P329G | AWNWIRQSPSRGLEWLGRTYYRSKWYNDYAVSVQ |
| | LALA) | GRITLIPDTSKNQFSLRLNSVTPEDTAVYYCASV |
| | | RAVAPFDYWGQGVLVTVSSASTKGPSVFPLAPSS |
| | | KSTSGGTAALGCLVEDYFPEPVTVSWNSGALTSG |
| | | VHTFPAVLQSSGLYSLSSVVTVPSSSLGTQTYIC |
| | | NVNHKPSNTKVDEKVEPKSCDGGGGSGGGGGQAV |
| | | VTQEPSLTVSPGGTVTLTCGSSTGAVTTSNYANW |
| | | VQEKPGQAFRGLIGGTNKRAPGTPARFSGSLLGG |
| | | KAALTLSGAQPEDEAEYYCALWYSNLWVFGGGTK |
| | | LTVLSSASTKGPSVFPLAPSSKSTSGGTAALGCL |
| | | VKDYFPEPVTVSWNSGALTSGVHTFPAVLQSSGL |
| | | YSLSSVVTVPSSSLGTQTYICNVNHKPSNTKVDK |
| | | KVEPKSCDKTHTCPPCPAPEAAGGPSVFLFPPKP |
| | | KDTLMISRTPEVTCVVVDVSHEDPEVKENWYVDG |
| | | VEVHNAKTKPREEQYNSTYRVVSVLTVLHQDWLN |
| | | GKEYKCKVSNKALGAPIEKTISKAKGQPREPQVY |
| | | TLPPCRDELTKNQVSLWCLVKGFYPSDIAVEWES |
| | | NGQPENNYKTTPPVLDSDGSFFLYSKLTVDKSRW |
| | | QQGNVFSCSVMHEALHNHYTQKSLSLSP |
| 58 | HLA-G VH-CH1(EE)-Fc | QVQLQQSGPGLLKPSQTLSLTCAISGDSVSSNRA |
| | (hole, P329G LALA) | AWNWIRQSPSRGLEWLGRTYYRSKWYNDYAVSVQ |
| | | GRITLIPDTSKNQFSLRLNSVTPEDTAVYYCASV |
| | | RAVAPFDYWGQGVLVTVSSASTKGPSVFPLAPSS |
| | | KSTSGGTAALGCLVEDYFPEPVTVSWNSGALTSG |
| | | VHTFPAVLQSSGLYSLSSVVTVPSSSLGTQTYIC |
| | | NVNHKPSNTKVDEKVEPKSCDKTHTCPPCPAPEA |
| | | AGGPSVFLFPPKPKDTLMISRTPEVTCVVVDVSH |
| | | EDPEVKFNWYVDGVEVHNAKTKPREEQYNSTYRV |
| | | VSVLTVLHQDWLNGKEYKCKVSNKALGAPIEKTI |
| | | SKAKGQPREPQVCTLPPSRDELTKNQVSLSCAVK |
| | | GFYPSDIAVEWESNGQPENNYKTTPPVLDSDGSF |
| | | FLVSKLTVDKSRWQQGNVFSCSVMHEALHNHYTQ |
| | | KSLSLSP |
| 59 | HLA-G VL-CL(RK) | DIVMTQSPDSLAVSLGERATINCKSSQSVLNPSN |
| | | NKNNLAWYQQQPGQPPKLLIYWASTRESGVPDRF |
| | | SGSGSGTDFTLTISSLQAEDVAVYFCQQYYRTPW |
| | | TFGQGTKVEIKRTVAAPSVFIFPPSDRKLKSGTA |
| | | SVVCLLNNFYPREAKVQWKVDNALQSGNSQESVT |
| | | EQDSKDSTYSLSSTLTLSKADYEKHKVYACEVTH |
| | | QGLSSPVTKSFNRGEC |
| 60 | CD3 VH-CL | EVQLLESGGGLVQPGGSLRLSCAASGFTFSSYAM |
| | | NWVRQAPGKGLEWVSRIRSKYNNYATYYADSVKG |
| | | RFTISRDDSKNTLYLQMNSLRAEDTAVYYCVRAS |
| | | NFPASYVSYFAYWGQGTLVTVSSASVAAPSVFIF |
| | | PPSDEQLKSGTASVVCLLNNFYPREAKVQWKVDN |
| | | ALQSGNSQESVTEQDSKDSTYSLSSTLILSKADY |
| | | EKHKVYACEVTHQGLSSPVTKSFNRGEC |
| 61 | exemplary human HLA-G | GSHSMRYFSAAVSRPGRGEPRFIAMGYVDDTQFV |
| | sequence | RFDSDSACPRMEPRAPWVEQEGPEYWEEETRNTK |
| | | AHAQTDRMNLQTLRGYYNQSEASSHTLQWMIGCD |
| | | LGSDGRLLRGYEQYAYDGKDYLALNEDLRSWTAA |
| | | DTAAQISKRKCEAANVAEQRRAYLEGTCVEWLHR |
| | | YLENGKEMLQRADPPKTHVTHHPVFDYEATLRCW |
| | | ALGFYPAEIILTWQRDGEDQTQDVELVETRPAGD |
| | | GTFQKWAAVVVPSGEEQRYTCHVQHEGLPEPLML |
| | | RWKQSSLPTIPIMGIVAGLVVLAAVVTGAAVAAV |
| | | LWRKKSSD |
| 62 | Atezolizumab Heavy chain | EVQLVESGGGLVQPGGSLRLSCAASGFTFSDSWI |
| | (P1AE0282) | HWVRQAPGKGLEWVAWISPYGGSTYYADSVKGRF |
| | | TISADTSKNTAYLQMNSLRAEDTAVYYCARRHWP |
| | | GGFDYWGQGTLVTVSSASTKGPSVFPLAPSSKST |
| | | SGGTAALGCLVKDYFPEPVTVSWNSGALTSGVHT |
| | | FPAVLQSSGLYSLSSVVTVPSSSLGTQTYICNVN |
| | | HKPSNTKVDKKVEPKSCDKTHTCPPCPAPELLGG |
| | | PSVFLFPPKPKDTLMISRTPEVTCVVVDVSHEDP |
| | | EVKFNWYVDGVEVHNAKTKPREEQYASTYRVVSV |
| | | LTVLHQDWLNGKEYKCKVSNKALPAPIEKTISKA |
| | | KGQPREPQVYTLPPSREEMTKNQVSLTCLVKGFY |
| | | PSDIAVEWESNGQPENNYKTTPPVLDSDGSFFLY |
| | | SKLTVDKSRWQQGNVFSCSVMHEALHNHYTQKSL |
| | | SLSP |
| 63 | Atezolizumab Light chain | DIQMTQSPSSLSASVGDRVTITCRASQDVSTAVA |
| | (P1AE0282) | WYQQKPGKAPKLLIYSASFLYSGVPSRFSGSGSG |
| | | TDFTLTISSLQPEDFATYYCQQYLYHPATFGQGT |
| | | KVEIKRTVAAPSVFIFPPSDEQLKSGTASVVCLL |
| | | NNFYPREAKVQWKVDNALQSGNSQESVTEQDSKD |
| | | STYSLSSTLTLSKADYEKHKVYACEVTHQGLSSP |
| | | VTKSFNRGEC |
| 64 | heavy chain variable | QMQLVQSGPEVKKPGTSVKVSCKASGYTFTDYNV |
| | domain VH, Favezelimab | DWVRQARGQRLEWIGDINPNDGGTIYAQKFQERV |
| | (MK 4280) | TITVDKSTSTAYMELSSLRSEDTAVYYCARNYRW |
| | | FGAMDHWGQGTTVTVSS |
| 65 | light chain variable domain | DIVMTQTPLSLSVTPGQPASISCKASQSLDYEGD |
| | VL Favezelimab (MK | SDMNWYLQKPGQPPQLLIYGASNLESGVPDRFSG |
| | 4280) | SGSGTDFTLKISRVEAEDVGVYYCQQSTEDPRTF |
| | | GGGTKVEIK |
| 66 | human CD3ϵ | UniProt accession no. P07766 |
| 67 | cynomolgus CD3ϵ | NCBI GenBank no. BAB71849.1 |
| 68 | human PD1 | UniProt accession no. Q15116 |
| 69 | exemplary human LAG3 | VPVVWAQEGAPAQLPCSPTIPLQDLSLLRRAGVT |
| | sequence (without signal | WQHQPDSGPPAAAPGHPLAPGPHPAAPSSWGPRP |
| | sequence) | RRYTVLSVGPGGLRSGRLPLQPRVQLDERGRQRG |
| | | DFSLWLRPARRADAGEYRAAVHLRDRALSCRLRL |
| | | RLGQASMTASPPGSLRASDWVILNCSFSRPDRPA |
| | | SVHWFRNRGQGRVPVRESPHHHLAESFLFLPQVS |
| | | PMDSGPWGCILTYRDGFNVSIMYNLTVLGLEPPT |
| | | PLTVYAGAGSRVGLPCRLPAGVGTRSFLTAKWTP |
| | | PGGGPDLLVTGDNGDFTLRLEDVSQAQAGTYTCH |
| | | IHLQEQQLNATVTLAIITVTPKSFGSPGSLGKLL |
| | | CEVTPVSGQERFVWSSLDTPSQRSFSGPWLEAQE |
| | | AQLLSQPWQCQLYQGERLLGAAVYFTELSSPGAQ |
| | | RSGRAPGALPAGHLLLFLILGVLSLLLLVTGAFG |
| | | FHLWRRQWRPRRFSALEQGIHPPQAQSKIEELEQ |
| | | EPEPEPEPEPEPEPEPEPEQL |
| 70 | human LAG3 Extracellular | VPVVWAQEGAPAQLPCSPTIPLQDLSLLRRAGVT |
| | Domain (ECD) | WQHQPDSGPPAAAPGHPLAPGPHPAAPSSWGPRP |
| | | RRYTVLSVGPGGLRSGRLPLQPRVQLDERGRQRG |
| | | DFSLWLRPARRADAGEYRAAVHLRDRALSCRLRL |
| | | RLGQASMTASPPGSLRASDWVILNCSFSRPDRPA |
| | | SVHWFRNRGQGRVPVRESPHHHLAESFLFLPQVS |
| | | PMDSGPWGCILTYRDGFNVSIMYNLTVLGLEPPT |
| | | PLTVYAGAGSRVGLPCRLPAGVGTRSFLTAKWTP |
| | | PGGGPDLLVTGDNGDFTLRLEDVSQAQAGTYTCH |
| | | IHLQEQQLNATVTLAIITVTPKSFGSPGSLGKLL |
| | | CEVTPVSGQERFVWSSLDTPSQRSFSGPWLEAQE |
| | | AQLLSQPWQCQLYQGERLLGAAVYFTELSSPGAQ |
| | | RSGRAPGALPAGHL |
| 71 | Peptide linker G₄S | GGGGS |
| 72 | Peptide linker (G₄S)₂ | GGGGSGGGGS |
| 73 | Peptide linker (G₄S)₃ | GGGGSGGGGSGGGGS |
| 74 | Peptide linker (G₄S)₄ | GGGGSGGGGSGGGGSGGGGS |
| 75 | Pembrolizumab | QVQLVQSGVEVKKPGASVKVSCKASGYTFTNYYM |
| | (P1AA6975) heavy chain | YWVRQAPGQGLEWMGGINPSNGGTNFNEKFKNRV |
| | | TLTTDSSTTTAYMELKSLQFDDTAVYYCARRDYR |
| | | FDMGFDYWGQGTTVTVSSASTKGPSVFPLAPCSR |
| | | STSESTAALGCLVKDYFPEPVTVSWNSGALTSGV |
| | | HTFPAVLQSSGLYSLSSVVTVPSSSLGTKTYTCN |
| | | VDHKPSNTKVDKRVESKYGPPCPPCPAPEFLGGP |
| | | SVFLFPPKPKDTLMISRTPEVTCVVVDVSQEDPE |
| | | VQFNWYVDGVEVHNAKTKPREEQFNSTYRVVSVL |
| | | TVLHQDWLNGKEYKCKVSNKGLPSSIEKTISKAK |
| | | GQPREPQVYTLPPSQEEMTKNQVSLTCLVKGFYP |
| | | SDIAVEWESNGQPENNYKTTPPVLDSDGSFFLYS |
| | | RLTVDKSRWQEGNVFSCSVMHEALHNHYTQKSLS |
| | | LSLG |
| 76 | Pembrolizumab | EIVLTQSPATLSLSPGERATLSCRASKGVSTSGY |
| | (P1AA6975) light chain | SYLHWYQQKPGQAPRLLIYLASYLESGVPARFSG |
| | | SGSGTDFTLTISSLEPEDFAVYYCQHSRDLPLTF |
| | | GGGTKVEIKRTVAAPSVFIFPPSDEQLKSGTASV |
| | | VCLLNNFYPREAKVQWKVDNALQSGNSQESVTEQ |
| | | DSKDSTYSLSSTLTLSKADYEKHKVYACEVTHQG |
| | | LSSPVTKSFNRGEC |
| 77 | Nivolumab heavy chain | QVQLVESGGGVVQPGRSLRLDCKASGITFSNSGM |
| | | HWVRQAPGKGLEWVAVIWYDGSKRYYADSVKGRF |
| | | TISRDNSKNTLFLQMNSLRAEDTAVYYCATNDDY |
| | | WGQGTLVTVSSASTKGPSVFPLAPCSRSTSESTA |
| | | ALGCLVKDYFPEPVTVSWNSGALTSGVHTFPAVL |
| | | QSSGLYSLSSVVTVPSSSLGTKTYTCNVDHKPSN |
| | | TKVDKRVESKYGPPCPPCPAPEFLGGPSVFLFPP |
| | | KPKDTLMISRTPEVTCVVVDVSQEDPEVQFNWYV |
| | | DGVEVHNAKTKPREEQFNSTYRVVSVLTVLHQDW |
| | | LNGKEYKCKVSNKGLPSSIEKTISKAKGQPREPQ |
| | | VYTLPPSQEEMTKNQVSLTCLVKGFYPSDIAVEW |
| | | ESNGQPENNYKTTPPVLDSDGSFFLYSRLTVDKS |
| | | RWQEGNVFSCSVMHEALHNHYTQKSLSLSLG |
| 78 | Nivolumab light chain | EIVLTQSPATLSLSPGERATLSCRASQSVSSYLA |
| | | WYQQKPGQAPRLLIYDASNRATGIPARFSGSGSG |
| | | TDFTLTISSLEPEDFAVYYCQQSSNWPRTFGQGT |
| | | KVEIKRTVAAPSVFIFPPSDEQLKSGTASVVCLL |
| | | NNFYPREAKVQWKVDNALQSGNSQESVTEQDSKD |
| | | STYSLSSTLTLSKADYEKHKVYACEVTHQGLSSP |
| | | VTKSFNRGEC |
| 79 | Anti-Lag3 heavy chain | QMQLVQSGPEVKKPGTSVKVSCKASGYTFTDYNV |
| | P1AD8676 Favezelimab | DWVRQARGQRLEWIGDINPNDGGTIYAQKFQERV |
| | (MK 4280) | TITVDKSTSTAYMELSSLRSEDTAVYYCARNYRW |
| | | FGAMDHWGQGTTVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNV |
| | | NHKPSNTKVDKKVEPKSCDKTHTCPPCPAPEAAG |
| | | GPSVFLFPPKPKDTLMISRTPEVTCVVVDVSHED |
| | | PEVKFNWYVDGVEVHNAKTKPREEQYNSTYRVVS |
| | | VLTVLHQDWLNGKEYKCKVSNKALGAPIEKTISK |
| | | AKGQPREPQVYTLPPSRDELTKNQVSLTCLVKGF |
| | | YPSDIAVEWESNGQPENNYKTTPPVLDSDGSFFL |
| | | YSKLTVDKSRWQQGNVFSCSVMHEALHNHYTQKS |
| | | LSLSP |
| 80 | Anti-Lag3 light chain | DIVMTQTPLSLSVTPGQPASISCKASQSLDYEGD |
| | P1AD8676 Favezelimab | SDMNWYLQKPGQPPQLLIYGASNLESGVPDRFSG |
| | (MK 4280) | SGSGTDFTLKISRVEAEDVGVYYCQQSTEDPRTF |
| | | GGGTKVEIKRTVAAPSVFIFPPSDEQLKSGTASV |
| | | VCLLNNFYPREAKVQWKVDNALQSGNSQESVTEQ |
| | | DSKDSTYSLSSTLTLSKADYEKHKVYACEVTHQG |
| | | LSSPVTKSFNRGEC |
| 81 | Anti-Lag3 heavy chain | QMQLVQSGPEVKKPGTSVKVSCKASGYTFTDYNV |
| | variable domain (VH | DWVRQARGQRLEWIGDINPNDGGTIYAQKFQERV |
| | P1AD8676) | TITVDKSTSTAYMELSSLRSEDTAVYYCARNYRW |
| | | FGAMDHWGQGTTVTVSS |
| 82 | Anti-Lag3 light chain | DIVMTQTPLSLSVTPGQPASISCKASQSLDYEGD |
| | variable domain VL | SDMNWYLQKPGQPPQLLIYGASNLESGVPDRFSG |
| | (P1AD8676) | SGSGTDFTLKISRVEAEDVGVYYCQQSTEDPRIF |
| | | GGGTKVEIK |
| 83 | heavy chain CDR-H1, CD3 | NYYIH |
| | (40G5c) | |
| 84 | heavy chain CDR-H2, CD3 | WIYPGDGNTKYNEKFKG |
| | (40G5c) | |
| 85 | heavy chain CDR-H3, CD3 | DSYSNYYFDY |
| | (40G5c) | |
| 86 | light chain CDR-L1, CD3 | KSSQSLLNSRTRKNYLA |
| | (40G5c) | |
| 87 | light chain CDR-L2, CD3 | WASTRES |
| | (40G5c) | |
| 88 | light chain CDR-L3, CD3 | TQSFILRT |
| | (40G5c) | |
| 89 | CD3 (40G5c) VH | EVQLVQSGAEVKKPGASVKVSCKASGYTFTNYYI |
| | | HWVRQAPGQGLEWIGWIYPGDGNTKYNEKFKGRA |
| | | TLTADTSTSTAYLELSSLRSEDTAVYYCARDSYS |
| | | NYYFDYWGQGTLVTVSS |
| 90 | CD3 (40G5c) VL | DIVMTQSPDSLAVSLGERATINCKSSQSLLNSRT |
| | | RKNYLAWYQQKPGQPPKLLIYWASTRESGVPDRF |
| | | SGSGSGTDFTLTISSLQAEDVAVYYCTQSFILRT |
| | | FGQGTKVEIK |
| 91 | heavy chain CDR-H1, | SNRAAWN |
| | HLA-G-0090-S33A | |
| 92 | heavy chain CDR-H2, | RTYYRSKWYNDYAVSVQG |
| | HLA-G-0090-S33A | |
| 93 | heavy chain CDR-H3, | VRAVAPFDY |
| | HLA-G-0090-S33A | |
| 94 | light chain CDR-L1, HLA- | KSSQSVLNSANNKNNLA |
| | G-0090-S33A | |
| 95 | light chain CDR-L2, HLA- | WASTRES |
| | G-0090-S33A | |
| 96 | light chain CDR-L3, HLA- | QQYYRTPWT |
| | G-0090-S33A | |
| 97 | HLA-G-0090-S33A VH | QVQLQQSGPGLLKPSQTLSLTCAISGDSVSSNRA |
| | | AWNWIRQSPSRGLEWLGRTYYRSKWYNDYAVSVQ |
| | | GRITLIPDTSKNQFSLRLNSVTPEDTAVYYCASV |
| | | RAVAPFDYWGQGVLVTVSS |
| 98 | HLA-G-0090-S33A-HCDR | DIVMTQSPDSLAVSLGERATINCKSSQSVLNPSN |
| | VL | NKNNLAWYQQQPGQPPKLLIYWASTRESGVPDRF |
| | | SGSGSGTDFTLTISSLQAEDVAVYFCQQYYRTPW |
| | | TFGQGTKVEIK |
| 99 | CD3 (40G5c) light chain | DIVMTQSPDSLAVSLGERATINCKSSQSLLNSRT |
| | | RKNYLAWYQQKPGQPPKLLIYWASTRESGVPDRF |
| | | SGSGSGTDFTLTISSLQAEDVAVYYCTQSFILRT |
| | | FGQGTKVEIKRTVAAPSVFIFPPSDEQLKSGTAS |
| | | VVCLLNNFYPREAKVQWKVDNALQSGNSQESVTE |
| | | QDSKDSTYSLSSTLTLSKADYEKHKVYACEVTHQ |
| | | GLSSPVTKSFNRGEC |
| 100 | CD3 (40G5c) heavy chain | EVQLVQSGAEVKKPGASVKVSCKASGYTFTNYYI |
| | | HWVRQAPGQGLEWIGWIYPGDGNTKYNEKFKGRA |
| | | TLTADTSTSTAYLELSSLRSEDTAVYYCARDSYS |
| | | NYYFDYWGQGTLVTVSSASTKGPSVFPLAPSSKS |
| | | TSGGTAALGCLVKDYFPEPVTVSWNSGALTSGVH |
| | | TFPAVLQSSGLYSLSSVVTVPSSSLGTQTYICNV |
| | | NHKPSNTKVDKKVEPKSCDKTHTCPPCPAPELLG |
| | | GPSVFLFPPKPKDTLMISRTPEVTCVVVDVSHED |
| | | PEVKFNWYVDGVEVHNAKTKPREEQYGSTYRVVS |
| | | VLTVLHQDWLNGKEYKCKVSNKALPAPIEKTISK |
| | | AKGQPREPQVYTLPPSREEMTKNQVSLSCAVKGF |
| | | YPSDIAVEWESNGQPENNYKTTPPVLDSDGSFFL |
| | | VSKLTVDKSRWQQGNVFSCSVMHEALHNHYTQKS |
| | | LSLSP |
| 101 | anti HLA-G (HC3B125) | QVQLQQSGPGLVKPSQTLSLTCAISGDSVSSNSA |
| | VH | AWNWIRQSPSRGLEWLGRTYYRSKWYNDYAVSVK |
| | | SRITINPDTSKNQISLQLNSVTPEDTAVYYCAGD |
| | | RRYGIVGLPFAYWGQGTLVTVSS |
| 102 | anti HLA-G (HC3B125) | DIVMTQSPDSLAVSLGERATINCKSSQSVLHSSN |
| | VL | NKNYLTWFQQKPGQPPKLLIYWASTRESGVPDRF |
| | | SGSGSGTDFTLTISSLQAEDVAVYYCHQYYSTPP |
| | | TFGQGTKVEIK |
| 103 | anti CD3 (HC3B 125) VH | QVQLQQSGPRLVRPSQTLSLTCAISGDSVENNNA |
| | | AWSWIRQSPSRGLEWLGRTYYRSKWLYDYAVSVK |
| | | SRITVNPDTSRNQFTLQLNSVTPEDTALYYCARG |
| | | YSSSFDYWGQGTLVTVSS |
| 104 | anti CD3 (HC3B125) VL | QSALTQPASVSGSPGQSITISCTGTSSNIGTYKF |
| | | VSWYQQHPDKAPKVLLYEVSKRPSGVSSRFSGSK |
| | | SGNTASLTISGLQAEDQADYHCVSYAGSGTLLFG |
| | | GGTKLTVL |
| 105 | KIEELE (part of LAG | KIEELE |
| | intracellular domain) | |
| 106 | exemplary human HLA-G | RIIPRHLQLG CGGSGGGGSG GGGSIQRTPK |
| | ß2M MHC class I complex | IQVYSRHPAE NGKSNFLNCY VSGFHPSDIE |
| | | VDLLKNGERI EKVEHSDLSF SKDWSFYLLY |
| | | YTEFTPTEKD EYACRVNHVT LSQPKIVKWD |
| | | RDMGGGGSGG GGSGGGGSGG GGSGSHSMRY |
| | | FSAAVSRPGR GEPRFIAMGY VDDTQFVRED |
| | | SDSACPRMEP RAPWVEQEGP EYWEEETRNT |
| | | KAHAQTDRMN LQTLRGCYNQ SEASSHTLQW |
| | | MIGCDLGSDG RLLRGYEQYA YDGKDYLALN |
| | | EDLRSWTAAD TAAQISKRKC EAANVAEQRR |
| | | AYLEGTCVEW LHRYLENGKE MLQRADPPKT |
| | | HVTHHPVFDY EATLRCWALG FYPAEIILTW |
| | | QRDGEDQTQD VELVETRPAG DGTFQKWAAV |
| | | VVPSGEEQRY TCHVQHEGLP EPLMLRWGSG |
| | | LNDIFEAQKI EWHEHHHHHH |

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered and referred to according to the numbering systems according to Kabat (Kabat, E. A., et al., Sequences of Proteins of Immunological Interest, 5th ed., Public Health Service, National Institutes of Health, Bethesda, MD (1991)) as defined above.

### Aspects of the Invention

In the following, some of the aspects of the invention are listed.
1. An anti-HLA-G/anti-CD3 bispecific antibody for use in a method of treating HLA-G-expressing cancer, wherein the anti-HLA-G/anti-CD3 bispecific antibody is used in combination with an anti-PD1/anti-LAG3 bispecific antibody.
2. The anti-HLA-G/anti-CD3 bispecific antibody for use in a method of paragraph (para) 1, wherein the anti-HLA-G/anti-CD3 bispecific antibody and the anti-PD1/anti-LAG3 bispecific antibody are administered together in a single composition or administered separately in two or more different compositions.
3. The anti-HLA-G/anti-CD3 bispecific antibody for use in a method of paras 1 or 2, wherein the anti-PD1/anti-LAG3 bispecific antibody comprises a Fc domain that is an IgG Fc domain, particularly an IgG1 Fc domain or an IgG4 Fc domain, and wherein the Fc domain comprises one or more amino acid substitution that reduces binding to an Fc receptor, in particular towards Fcγ receptor.
4. The anti-HLA-G/anti-CD3 bispecific antibody for use in a method of any one of paras 1 to 3, wherein the anti-PD1/anti-LAG3 bispecific antibody comprises an Fc domain of human IgG1 subclass with the amino acid mutations L234A, L235A and P329G (numbering according to Kabat EU index).
5. The anti-HLA-G/anti-CD3 bispecific antibody for use in a method of any one of paras 1 to 4, wherein the anti-PD1/anti-LAG3 bispecific antibody comprises a first antigen binding domain that specifically binds to programmed cell death protein 1 (PD1) and a second antigen binding domain that specifically binds to Lymphocyte activation gene-3 (LAG3), wherein the first antigen binding domain specifically binding to PD1 comprises a VH domain comprising
(i) CDR-H1 comprising the amino acid sequence of SEQ ID NO:1,
(ii) CDR-H2 comprising the amino acid sequence of SEQ ID NO:2, and
(iii) CDR-H3 comprising the amino acid sequence of SEQ ID NO:3; and
a VL domain comprising
(i) CDR-L1 comprising the amino acid sequence of SEQ ID NO:4;
(ii) CDR-L2 comprising the amino acid sequence of SEQ ID NO:5, and
(iii) CDR-L3 comprising the amino acid sequence of SEQ ID NO:6.
   6. The anti-HLA-G/anti-CD3 bispecific antibody for use in a method of any one of paras 1 to 5, wherein the anti-PD1/anti-LAG3 bispecific antibody comprises a second antigen binding domain that specifically binds to LAG3 comprising
(a) a VH domain comprising
   (i) CDR-H1 comprising the amino acid sequence of SEQ ID NO:11,
   (ii) CDR-H2 comprising the amino acid sequence of SEQ ID NO:12, and
   (iii) CDR-H3 comprising the amino acid sequence of SEQ ID NO:13; and
   a VL domain comprising
   (i) CDR-L1 comprising the amino acid sequence of SEQ ID NO:14,
   (ii) CDR-L2 comprising the amino acid sequence of SEQ ID NO:15, and
   (iii) CDR-L3 comprising the amino acid sequence of SEQ ID NO:16; or
   (b) a VH domain comprising
   (i) CDR-H1 comprising the amino acid sequence of SEQ ID NO:19,
   (ii) CDR-H2 comprising the amino acid sequence of SEQ ID NO:20, and
   (iii) CDR-H3 comprising the amino acid sequence of SEQ ID NO:21; and
   a VL domain comprising
   (i) CDR-L1 comprising the amino acid sequence of SEQ ID NO:22,
   (ii) CDR-L2 comprising the amino acid sequence of SEQ ID NO:23, and
   (iii) CDR-L3 comprising the amino acid sequence of SEQ ID NO:24.
   7. The anti-HLA-G/anti-CD3 bispecific antibody for use in a method of any one of paras 1 to 6, wherein the anti-PD1/anti-LAG3 bispecific antibody comprises a first antigen-binding domain specifically binding to PD1 comprising the VH domain comprising the amino acid sequence of SEQ ID NO:9 and the VL domain comprising the amino acid sequence of SEQ ID NO: 10.
   8. The anti-HLA-G/anti-CD3 bispecific antibody for use in a method of any one of paras 1 to 7, wherein the anti-PD1/anti-LAG3 bispecific antibody comprises a second antigen-binding domain specifically binding to LAG3 comprising
(a) a VH domain comprising the amino acid sequence of SEQ ID NO:17 and a VL domain comprising the amino acid sequence of SEQ ID NO:18, or
(b) a VH domain comprising the amino acid sequence of SEQ ID NO:25 and a VL domain comprising the amino acid sequence of SEQ ID NO:26.
   9. The anti-HLA-G/anti-CD3 bispecific antibody for use in a method of any one of paras 1 to 5 or 7, wherein the anti-PD1/anti-LAG3 bispecific antibody comprises a second antigen-binding domain specifically binding to LAG3 comprising
(a) a VH domain comprising the amino acid sequence of SEQ ID NO:27 and a VL domain comprising the amino acid sequence of SEQ ID NO:28, or
(b) a VH domain comprising the amino acid sequence of SEQ ID NO:29 and a VL domain comprising the amino acid sequence of SEQ ID NO:30, or
(c) a VH domain comprising the amino acid sequence of SEQ ID NO:31 and a VL domain comprising the amino acid sequence of SEQ ID NO:32, or
(d) a VH domain comprising the amino acid sequence of SEQ ID NO:33 and a VL domain comprising the amino acid sequence of SEQ ID NO:34, or
(e) a VH domain comprising the amino acid sequence of SEQ ID NO:64 and a VL domain comprising the amino acid sequence of SEQ ID NO:65.
   10. The anti-HLA-G/anti-CD3 bispecific antibody for use in a method of any one of paras 1 to 8, wherein the anti-PD1/anti-LAG3 bispecific antibody comprises
a first antigen binding domain specifically binding to PD1 comprising a VH domain comprising the amino acid sequence of SEQ ID NO:9 and a VL domain comprising the amino acid sequence of SEQ ID NO:10,
and a second antigen binding domain specifically binding to LAG3 comprising a VH domain comprising the amino acid sequence of SEQ ID NO:17 and a VL domain comprising the amino acid sequence of SEQ ID NO:18.
   11. The anti-HLA-G/anti-CD3 bispecific antibody for use in a method of any one of paras 1 to 10, wherein the anti-PD1/anti-LAG3 bispecific antibody comprises a Fab fragment specifically binding to PD1 and a Fab fragment specifically binding to LAG3.
   12. The anti-HLA-G/anti-CD3 bispecific antibody for use in a method of any one of paras 1 to 11, wherein the anti-PD1/anti-LAG3 bispecific antibody comprises a Fab fragment specifically binding to PD1, wherein the variable domains VL and VH are replaced by each other so that VL is part of the heavy chain and VH is part of the light chain.
   13. The anti-HLA-G/anti-CD3 bispecific antibody for use in a method of any one of paras 1 to 12, wherein the anti-PD1/anti-LAG3 bispecific antibody comprises monovalent binding to PD1 and monovalent binding to LAG3.
   14. The anti-HLA-G/anti-CD3 bispecific antibody for use in a method of any one of paras 1 to 8 and 10 to 13, wherein the anti-PD1/anti-LAG3 bispecific antibody comprises
(a) a first heavy chain comprising an amino acid sequence of SEQ ID NO:35, a first light chain comprising an amino acid sequence of SEQ ID NO:36, a second heavy chain comprising an amino acid sequence of SEQ ID NO:37, and a second light chain comprising an amino acid sequence of SEQ ID NO:38, or
(b) a first heavy chain comprising an amino acid sequence of SEQ ID NO:35, a first light chain comprising an amino acid sequence of SEQ ID NO:36, a second heavy chain comprising an amino acid sequence of SEQ ID NO:39, and a second light chain comprising an amino acid sequence of SEQ ID NO:40.
   15. The anti-HLA-G/anti-CD3 bispecific antibody for use in a method of any one of paras 1 to 8 and 10 to 14, wherein the anti-PD1/anti-LAG3 bispecific antibody comprises a first heavy chain comprising an amino acid sequence of SEQ ID NO:35, a first light chain comprising an amino acid sequence of SEQ ID NO:36, a second heavy chain comprising an amino acid sequence of SEQ ID NO:37, and a second light chain comprising an amino acid sequence of SEQ ID NO:38.
   16. The anti-HLA-G/anti-CD3 bispecific antibody for use in a method of any one of paras 1 to 15, wherein the anti-HLA-G/anti-CD3 bispecific antibody comprises a first antigen binding domain that specifically binds to CD3 comprising a heavy chain variable region (V_{H}CD3) and a light chain variable region (V_{L}CD3), and a second antigen binding domain that specifically binds to HLA-G comprising a heavy chain variable region (V_{H}HLA-G) and a light chain variable region (V_{L}HLA-G).
   17. The anti-HLA-G/anti-CD3 bispecific antibody for use in a method of any one of paras 1 to 15, wherein the anti-HLA-G/anti-CD3 bispecific antibody comprises a first antigen binding domain that specifically binds to CD3 and a second antigen binding domain that specifically binds to HLA-G, wherein the first antigen binding domain specifically binding to CD3 comprises a VH domain comprising
   (i) CDR-H1 comprising the amino acid sequence of SEQ ID NO:41,
   (ii) CDR-H2 comprising the amino acid sequence of SEQ ID NO:42, and
   (iii) CDR-H3 comprising the amino acid sequence of SEQ ID NO:43; and
   a VL domain comprising
   (i) CDR-L1 comprising the amino acid sequence of SEQ ID NO:44,
   (ii) CDR-L2 comprising the amino acid sequence of SEQ ID NO:45, and
   (iii) CDR-L3 comprising the amino acid sequence of SEQ ID NO:46.
   18. The anti-HLA-G/anti-CD3 bispecific antibody for use in a method of any one of paras 1 to 17, wherein the first antigen binding domain specifically binding to CD3 comprises a VH domain comprising the amino acid sequence of SEQ ID NO:47 and a VL domain comprising the amino acid sequence of SEQ ID NO:48.
   19. The anti-HLA-G/anti-CD3 bispecific antibody for use in a method of any one of paras 1 to 18, wherein the second antigen binding domain specifically binding to HLA-G comprises
   a) a VH domain comprising
   i) CDR-H1 comprising the amino acid sequence of SEQ ID NO:49,
   ii) CDR-H2 comprising the amino acid sequence of SEQ ID NO:50, and
   iii) CDR-H3 comprising the amino acid sequence of SEQ ID NO:51, and
   a VL domain comprising
   i) CDR-L1 comprising the amino acid sequence of SEQ ID NO:52,
   ii) CDR-L2 comprising the amino acid sequence of SEQ ID NO:53, and
   iii) CDR-L3 comprising the amino acid sequence of SEQ ID NO:54, or
   b) a VH domain comprising
   i) CDR-H1 comprising the amino acid sequence of SEQ ID NO:91,
   ii) CDR-H2 comprising the amino acid sequence of SEQ ID NO:92, and
   iii) CDR-H3 comprising the amino acid sequence of SEQ ID NO:93, and
   a VL domain comprising
   i) CDR-L1 comprising the amino acid sequence of SEQ ID NO:94,
   ii) CDR-L2 comprising the amino acid sequence of SEQ ID NO:95, and
   iii) CDR-L3 comprising the amino acid sequence of SEQ ID NO:95.
   20. The anti-HLA-G/anti-CD3 bispecific antibody for use in a method of any one of paras 1 to 19, wherein the anti-HLA-G/anti-CD3 bispecific antibody comprises a second antigen-binding domain specifically binding to HLA-G comprising
(a) a VH domain comprising the amino acid sequence of SEQ ID NO:55 and a VL domain comprising the amino acid sequence of SEQ ID NO:56, or
   (b) a VH domain comprising the amino acid sequence of SEQ ID NO:97 and a VL domain comprising the amino acid sequence of SEQ ID NO:98.
   21. The anti-HLA-G/anti-CD3 bispecific antibody for use in a method of any one of paras 1 to 20, wherein the anti-HLA-G/anti-CD3 bispecific antibody comprises a third antigen binding domain that binds to HLA-G.
   22. The anti-HLA-G/anti-CD3 bispecific antibody for use in a method of any one of paras 1 to 21, wherein the anti-HLA-G/anti-CD3 bispecific antibody comprises an Fc domain comprising one or more amino acid substitutions that reduce binding to an Fc receptor and/or effector function.
   23. The anti-HLA-G/anti-CD3 bispecific antibody for use in a method of any one of paras 1 to 22, wherein the anti-HLA-G/anti-CD3 bispecific antibody comprises a first heavy chain comprising an amino acid sequence of SEQ ID NO:57, a second heavy chain comprising an amino acid sequence of SEQ ID NO:58, a first light chain comprising an amino acid sequence of SEQ ID NO: 59, and a second light chain comprising an amino acid sequence of SEQ ID NO:60.
   24. The anti-HLA-G/anti-CD3 bispecific antibody for use in a method of any one of paras 1 to 23, wherein the anti-HLA-G/anti-CD3 bispecific antibody is used in combination with anti-PD1/anti-LAG3 bispecific antibody and wherein the combination is for administration at intervals from about about one week to three weeks.
   25. A pharmaceutical composition comprising a combination of an anti-HLA-G/anti-CD3 bispecific antibody and an anti-PD1/anti-LAG3 bispecific antibody for use in the combined, sequential or simultaneous, treatment of a disease, in particular cancer, particularly an HLA-G expressing cancer.
   26. A pharmaceutical composition comprising an anti-HLA-G/anti-CD3 bispecific antibody and a pharmaceutically acceptable carrier, and a second medicament comprising an anti-PD1/anti-LAG3 bispecific antibody and optionally a pharmaceutically acceptable carrier.
   27. The pharmaceutical composition of para 26 for use in the treatment of HLA-G expressing cancer, in particular a cancer selected from lung cancer, non small cell lung (NSCL) cancer, bronchioloalveolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, carcinoma of the fallopian tubes, carcinoma of the endometrium, carcinoma of the cervix, carcinoma of the vagina, carcinoma of the vulva, Hodgkin's Disease, nasopharyngeal carcinoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymomas, medulloblastomas, meningiomas, squamous cell carcinomas, pituitary adenoma, lymphoma, lymphocytic leukemia, including refractory versions of any of the above cancers, or a combination of one or more of the above cancers.
   28. Use of a combination of an anti-HLA-G/anti-CD3 bispecific antibody and an anti-PD1/anti-LAG3 bispecific antibody in the manufacture of a medicament for treating cancer, in particular an HLA-G expressing cancer.
   29. A method for treating cancer, in particular an HLA-G expressing cancer, in a subject comprising administering to the subject an effective amount of an anti-HLA-G/anti-CD3 antibody and an effective amount of an anti-PD1/anti-LAG3 bispecific antibody.
   30. The method of para 29, wherein the anti-HLA-G/anti-CD3 bispecific antibody and the anti-PD1/anti-LAG3 bispecific antibody are administered together in a single composition or administered separately in two or more different compositions.
   31. The method of paras 29 or 30, wherein the anti-HLA-G/anti-CD3 bispecific antibody and the anti-PD1/anti-LAG3 bispecific antibody are administered intravenously or subcutaneously.
   32. The method of any one of paras 29 to 31, wherein the anti-HLA-G/anti-CD3 bispecific antibody is administered concurrently with, prior to, or subsequently to the anti-PD1/anti-LAG3 bispecific antibody.
   33. An anti-HLA-G/anti-CD3 bispecific antibody for use in a method of treating HLA-G-expressing cancer, wherein the anti-HLA-G/anti-CD3 bispecific antibody is used in combination with an anti-PD1/anti-LAG3 bispecific antibody, wherein the anti-PD1/anti-LAG3 bispecific antibody comprises a first antigen binding domain that specifically binds to PD1 and a second antigen binding domain that specifically binds to LAG3,
   wherein the first antigen binding domain specifically binding to PD1 comprises a VH domain comprising (i) CDR-H1 comprising the amino acid sequence of SEQ ID NO:1. (ii) CDR-H2 comprising the amino acid sequence of SEQ ID NO:2, and (iii) CDR-H3 comprising the amino acid sequence of SEQ ID NO:3; and a VL domain comprising (i) CDR-L1 comprising the amino acid sequence of SEQ ID NO:4; (ii) CDR-L2 comprising the amino acid sequence of SEQ ID NO: 5, and (iii) CDR-L3 comprising the amino acid sequence of SEQ ID NO:6, and
   wherein the second antigen binding domain that specifically binds to LAG3 comprises a VH domain comprising (i) CDR-H1 comprising the amino acid sequence of SEQ ID NO:11, (ii) CDR-H2 comprising the amino acid sequence of SEQ ID NO:12, and (iii) CDR-H3 comprising the amino acid sequence of SEQ ID NO: 13; and a VL domain comprising (i) CDR-L1 comprising the amino acid sequence of SEQ ID NO:14, (ii) CDR-L2 comprising the amino acid sequence of SEQ ID NO: 15, and (iii) CDR-L3 comprising the amino acid sequence of SEQ ID NO:16; and
   wherein the anti-HLA-G/anti-CD3 bispecific antibody comprises a first antigen binding domain that specifically binds to CD3 and a second antigen binding domain that specifically binds to HLA-G,
   wherein the first antigen binding domain specifically binding to CD3 comprises a VH domain comprising (i) CDR-H1 comprising the amino acid sequence of SEQ ID NO:41, (ii) CDR-H2 comprising the amino acid sequence of SEQ ID NO:42, and (iii) CDR-H3 comprising the amino acid sequence of SEQ ID NO:43; and a VL domain comprising (i) CDR-L1 comprising the amino acid sequence of SEQ ID NO:44, (ii) CDR-L2 comprising the amino acid sequence of SEQ ID NO: 45, and (iii) CDR-L3 comprising the amino acid sequence of SEQ ID NO:46, and
   wherein the second antigen binding domain specifically binding to HLA-G comprises a VH domain comprising i) CDR-H1 comprising the amino acid sequence of SEQ ID NO:49, ii) CDR-H2 comprising the amino acid sequence of SEQ ID NO:50, and iii) CDR-H3 comprising the amino acid sequence of SEQ ID NO:51, and a VL domain comprising i) CDR-L1 comprising the amino acid sequence of SEQ ID NO:52, ii) CDR-L2 comprising the amino acid sequence of SEQ ID NO: 53, and iii) CDR-L3 comprising the amino acid sequence of SEQ ID NO:54.
   34. An anti-HLA-G/anti-CD3 bispecific antibody for use in a method of treating HLA-G-expressing cancer, wherein the anti-HLA-G/anti-CD3 bispecific antibody is used in combination with an anti-PD1/anti-LAG3 bispecific antibody,
   wherein the anti-PD1/anti-LAG3 bispecific antibody comprises a first antigen binding domain that specifically binds to PD1 and a second antigen binding domain that specifically binds to LAG3,
   wherein the first antigen-binding domain specifically binding to PD1 comprises the VH domain comprising the amino acid sequence of SEQ ID NO:9 and the VL domain comprising the amino acid sequence of SEQ ID NO:10,
   wherein the second antigen-binding domain specifically binding to LAG3 comprising a VH domain comprising the amino acid sequence of SEQ ID NO: 17 and a VL domain comprising the amino acid sequence of SEQ ID NO:18, and
   wherein the anti-HLA-G/anti-CD3 bispecific antibody comprises a first antigen binding domain that specifically binds to CD3 and a second antigen binding domain that specifically binds to HLA-G,
   wherein the first antigen binding domain specifically binding to CD3 comprises a VH domain comprising the amino acid sequence of SEQ ID NO:47 and a VL domain comprising the amino acid sequence of SEQ ID NO:48, and
   wherein second antigen-binding domain specifically binding to HLA-G comprises a VH domain comprising the amino acid sequence of SEQ ID NO:55 and a VL domain comprising the amino acid sequence of SEQ ID NO:56.

### EXAMPLES

### Recombinant DNA Techniques

Standard methods were used to manipulate DNA as described in Sambrook et al., Molecular cloning: A laboratory manual; Cold Spring Harbor Laboratory Press, Cold Spring Harbor, New York, 1989. The molecular biological reagents were used according to the manufacturer's instructions. General information regarding the nucleotide sequences of human immunoglobulin light and heavy chains is given in: Kabat, E. A. et al., (1991) Sequences of Proteins of Immunological Interest, Fifth Ed., NIH Publication No 91-3242.

### DNA Sequencing

DNA sequences were determined by double strand sequencing.

### Gene Synthesis

Desired gene segments were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells.

### Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

### Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

### SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGER Antioxidant running buffer additive) or MOPS (non-reduced gels) running buffer was used.

### Analytical Size Exclusion Chromatography

Size exclusion chromatography (SEC) for the determination of the aggregation and oligomeric state of antibodies was performed by HPLC chromatography. Briefly, Protein A purified antibodies were applied to a Tosoh TSKgel G3000SW column in 300 mM NaCl, 50 mM KH₂PO₄/K₂HPO₄, pH 7.5 on an Agilent HPLC 1100 system or to a Superdex 200 column (GE Healthcare) in 2×PBS on a Dionex HPLC-System. The eluted protein was quantified by UV absorbance and integration of peak areas. BioRad Gel Filtration Standard 151-1901 served as a standard.

### Example 1

### Preparation, Purification and Characterization of Antibodies Used in the Experiments

### a) HLA-G TCB

HLA-G TCB molecules have been prepared according to the methods described in WO 2022/129120 A1. The anti-HLA-G/anti-CD3 bispecific antibody used in the following experiments corresponds to HLA-G-0090-VL-S32P as described in Example 2 of WO 2022/129120 A1 (in the following also referred to as “P1AF7977” or HLA-G TCB). P1AF7977 is a “2+1 IgG CrossFab” antibody and is comprised of two different heavy chains and two different light chains which comprise the amino acid sequences of SEQ ID NO:57, SEQ ID NO: 58, SEQ ID NO:59 and SEQ ID NO:60 (see also Table B). 2+1 means that the molecule has two antigen binding domains specific for HLA-G and one antigen binding domain specific for CD3. Point mutations in the CH3 domain (“knobs into holes”) were introduced to promote the assembly of the two differing heavy chains. The Pro329Gly, Leu234Ala and Leu235Ala mutations (“PGLALA”) were introduced in the constant region of the knob and hole heavy chains to abrogate binding to Fc gamma receptors according to the method described in WO 2012/130831. Exchange of the VH and VL domains in the CD3 binding Fab and point mutations in the CH1 and CL domains in the HLA-G binding Fab were made in order to promote the correct assembly of the two different light chains. The molecule is further characterized in Example 2 ff of WO 2022/129120 A1. A schematic representation of the bispecific antibody in 2+1 CrossFab format is shown in FIG. 1A.

### b) Anti-PD1/Anti-LAG3 Antibodies

Bispecific antibodies which bind to human PD1 and human LAG3 with VH/VL domain exchange/replacement (CrossMAb^{Vh-VL}) in one binding arm were generated as described in Example 10.1 of WO 2018/185043. The preparation of multispecific 1+1 CrossMAb^{Vh-VI} antibodies is also described in WO 2009/080252.

The anti-PD1/anti-LAG3 antibody P1AA0927 comprises the amino acid sequences of SEQ ID NO:35, SEQ ID NO:36, SEQ ID NO:37 and SEQ ID NO:38. The bispecific antibodies were expressed using expression plasmids containing the nucleic acids encoding the above amino acid sequences. Knobs into holes heterodimerization technology was used with a typical knob (T366W) substitution in the first CH3 domain and the corresponding hole substitutions (T366S, L368A and Y410V) in the second CH3 domain (as well as two additional introduced cysteine residues S354C/Y349′C) (contained in the respective corresponding heavy chain (HC) sequences depicted above). The Pro329Gly, Leu234Ala and Leu235Ala mutations (“PGLALA”) were introduced in the constant region of the knob and hole heavy chains to abrogate binding to Fc gamma receptors according to the method described in WO 2012/130831. In order to improve correct pairing, amino acid substitutions were additionally introduced in the CH and CL domain of the conventional Fab (charged variants). A schematic representation of the bispecific anti-PD1/anti-LAG3 antibody in 1+1 CrossMAb^{Vh-VI} format is shown in FIG. 1B.

The bispecific antibody expressed as above was purified from the supernatant by a combination of Protein A affinity chromatography and size exclusion chromatography. The obtained products were characterized for identity by mass spectrometry and analytical properties such as purity by SDS-PAGE, monomer content and stability.

### c) Reference Antibodies

The anti-PD-L1 antibody Atezolizumab (herein also referred to as “P1AE0828”) used for comparison is described in WO2010/077634 and comprises a heavy chain comprising the amino acid sequence of SEQ ID NO: 62 and a light chain comprising the amino acid sequence of SEQ ID NO: 63)

The anti-PD1 antibody Pembrolizumab (herein also referred to as “P1AA6975”) used for comparison is described in WO 2008/156712 and comprises the VH domain comprising the amino acid sequence of SEQ ID NO:9 and the VL domain comprising the amino acid sequence of SEQ ID NO: 10.

The anti-LAG3 antibody Favezelimab (herein also referred to as “P1AD8676”) used for comparison is described in WO 2016/028672 and comprises the VH domain comprising the amino acid sequence of SEQ ID NO:64 and the VL domain comprising the amino acid sequence of SEQ ID NO: 65.

### Example 2

### Potent Anti-Tumor Effect of the Combination Therapy of PD1/LAG3 Bispecific Antibodies and HLA-G TCB In Vivo in the BC004 PDX Model in Humanized NSG Mice

The anti-tumor activity of anti-PD-1/anti-LAG3 bispecific antibody P1AA0927 in combination with anti-HLA-G/anti-CD3 bispecific antibody (HLA-G TCB; P1AF7977) was assessed in vivo in HSC-NSG mice (i.e. immunodeficient mice humanized by engraftment with human hematopoietic stem cells (“HSC”)) engrafted with the human orthotopic breast PDX (patient-derived xenograft) model BC004, injected into the intramammary fat pad. The efficacy of this combination was compared to single treatment with HLA-G CD3 TCB, to single treatment with atezolizumab (P1AE0282) and to a combination of Atezolizumab with HLA-G CD3 TCB.

### a) Experimental Material and Methods

Preparation of BC004 cells: The human breast cancer PDX model BC004 was purchased from OncoTest (Freiburg, Germany). Tumor fragments were digested with collagenase D (Catalog No. 11088882001; Sigma-Aldrich; Minneapolis, MI) and DNase I (Catalog No. 10104159001; Sigma-Aldrich) for 20 minutes to prepare single-cell suspensions. Cell number and viability were determined using a ViCell counter (Beckman Coulter; Brea, CA).

Production of fully humanized mice: Female 3-week old NSG (NOD/scid/IL-2Rynull) mice were irradiated (140 cGy) and engrafted by intravenous injection of 9×10+CD34+ cord blood cells per mouse at Jackson Laboratories. After reaching a human immune infiltrate (hCD45) above 25% in blood, mice were shipped to Roche and maintained for 5 days to get accustomed to the new environment. Mice were kept under specific-pathogen-free condition with daily cycles of 12 h light/12 h darkness according to committed guidelines (GV-Solas; Felasa; TierschG). Continuous health monitoring was carried out on a daily basis. Experimental study protocol was reviewed and approved by local government (ROB-55.2-2532.Vet_03-20-170).

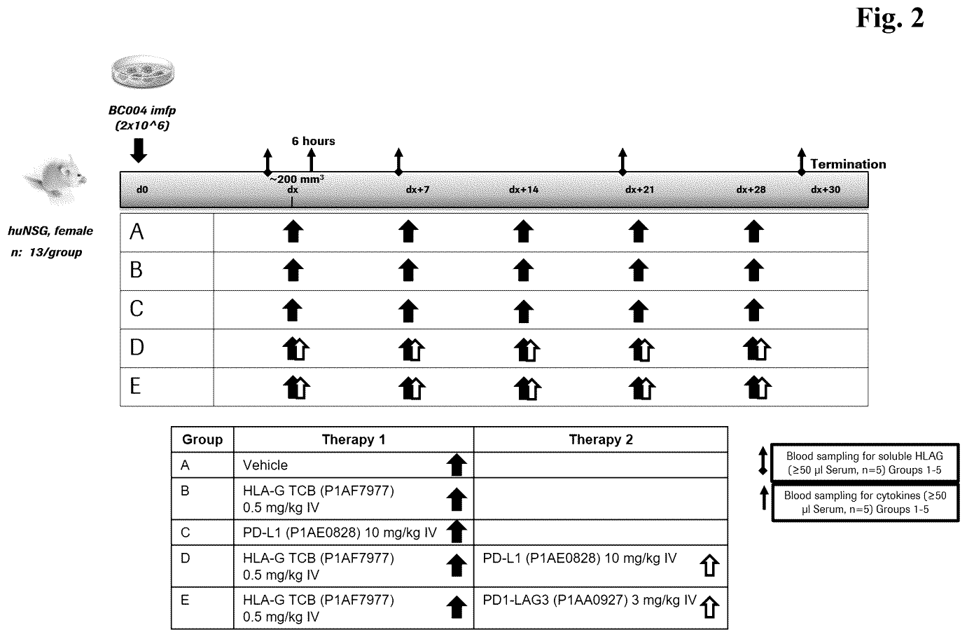

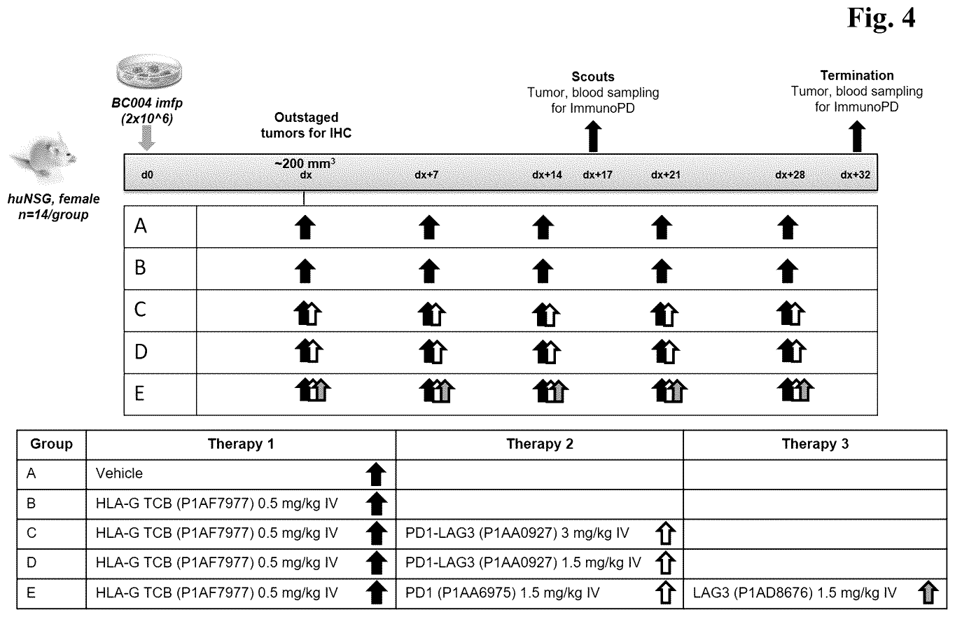

Efficacy Experiment: Humanized mice were injected with 2×10⁶ BC004 cells in a total volume of 20 μL PBS into the mammary fat pad (i.m.f.p.). Once tumors reached an average volume of approximately 200 mm³, mice were randomized into different treatment groups based on tumor volume. The first group of mice received histidine buffer (vehicle) as control. All antibodies were prepared freshly in 20 mM Histidine, 140 mM NaCl, pH 6.0 before injection and administered weekly, intravenously (IV) at the doses indicated in FIG. 2 (Study #1173) or FIG. 4 (Study #1655), respectively. Animals were controlled daily for clinical symptoms and detection of adverse effects. Tumor volume was measured by caliper and body weight was controlled twice weekly. Tumor volume was calculated as followed:

$${T}_{v} : ( {W}^{2} / 2 ) \times L ( W : Width , L : Length )$$

Termination criteria for animals were visible sickness (scruffy fur, arched back, breathing problems, impaired locomotion), body weight loss (≥25% within 7 days after first treatment or ≥20% after 7 days of treatment) or tumor size (diameter ≥2 cm, volume ≥4000 mm³). Animals were sacrificed according to the termination criteria or at the end of the experiment.

The impact of the therapy was assessed by measuring the tumor size and displayed as tumor growth over time either as median, or as as tumor growth over time for each single mouse. Animals with tumors only of a tumor volume of less than 50 mm³ wer considered as tumor-free animals.

To evaluate the immunopharmacodynamic changes induced by P1AF7977 treatment as monotherapy and as combination therapy, flow cytometry analysis was performed in study #1655 on treatment Day 17 (3 days after the third treatment cycle) and termination (4 days after the fifth treatment cycle). Tumors were harvested and processed to obtain a single-cell suspension. Samples were disrupted using manual scissors and the Miltenyi Gentle MACS apparatus and were then digested in an enzyme mix containing DNAsel (Catalog No. 10104159001; Sigma-Aldrich) and Collagenase D (Catalog No. 11088882001; Sigma-Aldrich). After digestion, the tissue mix was filtered through a 100- and 70-μm filter and resuspended to a single-cell suspension in buffer containing a cocktail of fluorescently labeled antibodies for detection of immune cells and tumor targets. Stained cells were analyzed on a Cytek Aurora 5L (Cytek Biosciences). Raw data were analyzed using FlowJo software and a predefined gating strategy to identify tumor-infiltrating lymphocytes.

Graphical analysis was performed using GraphPad Prism v6.07 (GraphPad Software, Inc) or TIBCO Spotfire. Statistical analysis was performed using the internal software DOPSa. Study groups were tested using the non-parametric multiple comparison test (Dunn's method). Significant changes compared to the control group (vehicle) with p<0.05 were depicted with asterisks (*p<0.05. **p<0.01, ***p<0.001).

To assess histological changes after treatment with P1AF7977 monotherapy and combination with P1AE0282 and P1AA0927, tumors of treatment day 3 and termination were removed and stored in formalin for 24 hours. Formalin-fixed, paraffin-embedded tissue (FFPET) sections were stained for HLA-G (clone 4H84, abcam) as well as huCD3 (clone 2GV6, Ventana).

### b) Results of Study #1173

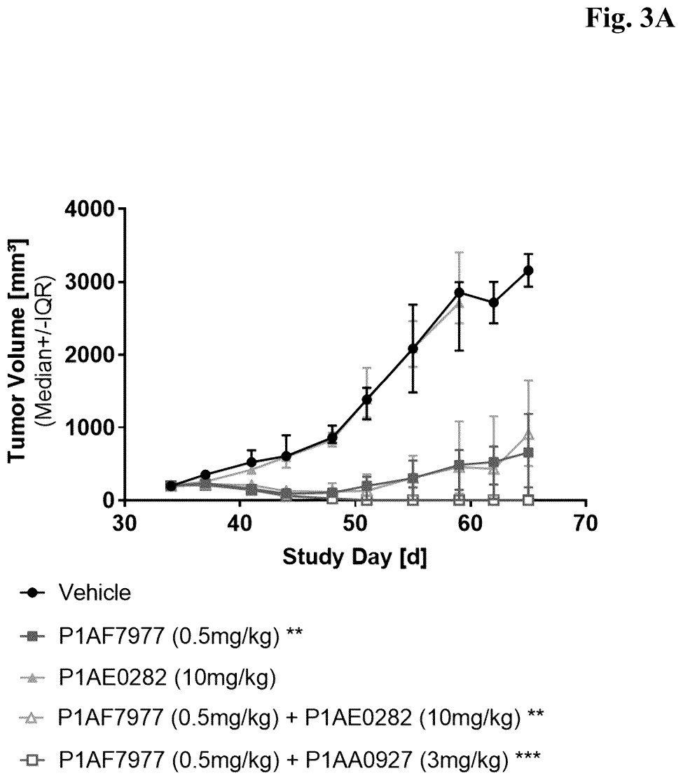

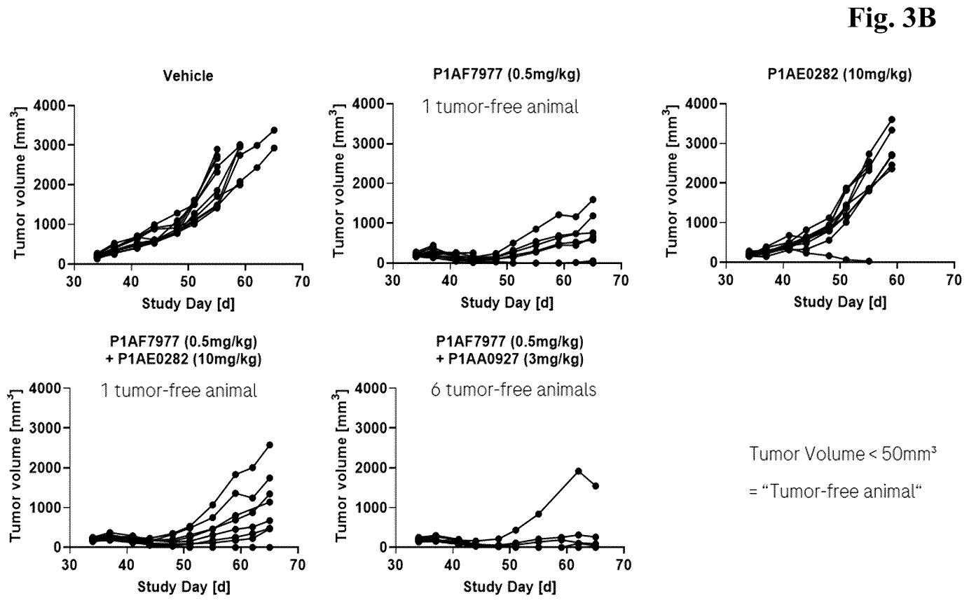

HLA-G TCB (P1AF7977) at a concentration of 0.5 mg/kg shows an anti-tumor response in BC004 tumor-bearing huNSG animals, as demonstrated by a decrease in tumor volume (FIGS. 3A and 3B). The response in animals treated with an anti-PD-L1 antibody monotherapy (P1AE0282) at 10 mg/kg did not differ from that of BC004 vehicle tumors in the current model. The combination of P1AE0282 treatment with P1AF7977 treatment did not lead to an increase in the response, compared to P1AF7977 monotheray. The therapeutic efficacy of P1AF7977 can be increased considerably, however, when combined with P1AA0927 at concentrations of 3 mg/kg.

### c) Results of Study #1655

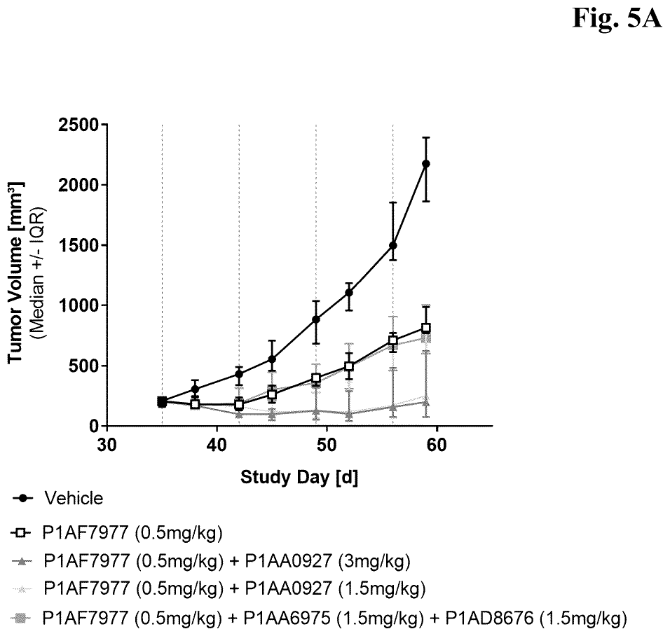

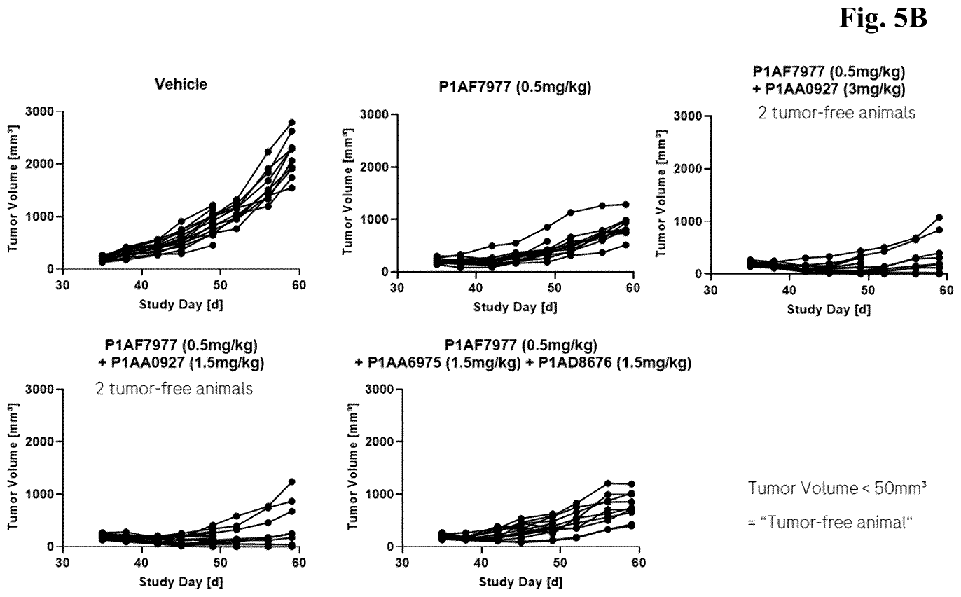

As in the previous study, P1AF7977 shows an anti-tumor response at a concentration of 0.5 mg/kg in BC004 tumor-bearing huNSG animals, as demonstrated by a decrease in tumor volume (FIGS. 5A and 5B). The therapeutic efficacy of P1AF7977 was not increased by combination with a treatment with anti-PD1 antibody and anti-LAG3 antibody at 1.5 mg/kg each. In contrast, the therapeutic efficacy of PIAF7977 can be increased with a combination with P1AA0927. Combinations of P1AF7977 at 0.5 mg/kg with with P1AA00927 at concentrations of 1.5 mg/kg and 3 mg/kg (FIGS. 5A and 5B) showed comparable levels of increase of efficacy.

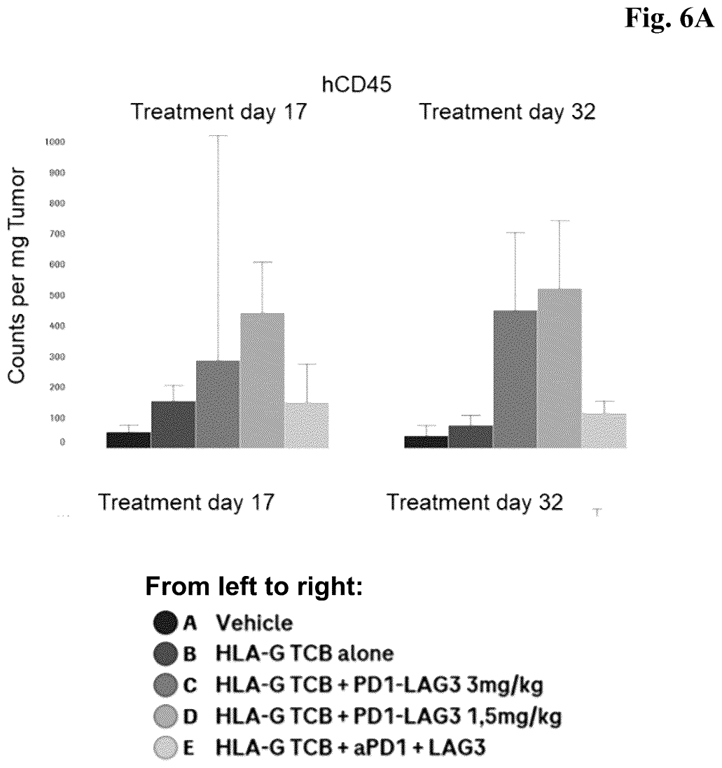

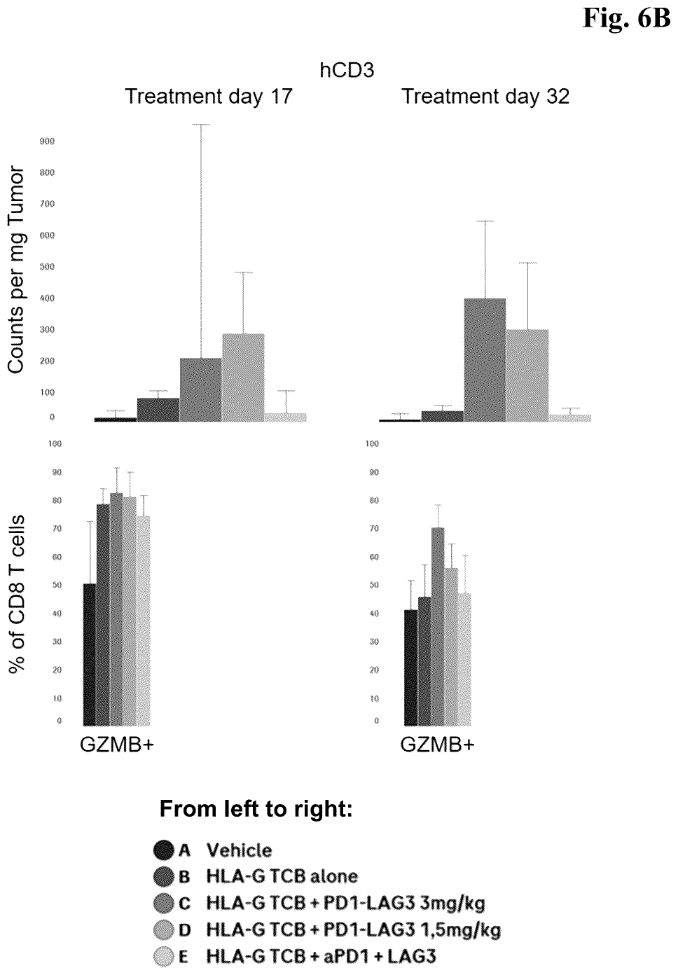

Immunopharmacodynamic analysis of T cell infiltration in BC004 tumors on day 17 and day 32 after treatment confirmed higher immune cell infiltration (characterized by markers CD45+ (FIG. 6A) or CD3+ (FIG. 6B) in the tumor after P1AF7977 monotherapy (on day 17) and combination with P1AA0927 (on treatment day 17 and 32), indicating enhanced immune cell infiltration after combination therapy.

On treatment day 32, the infiltration with immune cells (FIG. 6A, right) and T cells in particular (FIG. 6B, top right) was significantly elevated in tumors treated with a combination of HLA-G TCB (P1AF7977) at 0.5 mg/kg and anti-PD1/anti-LAG3 bispecific antibody (PD1-LAG3)—both at concentrations of 1.5 mg/kg and 3 mg/kg-compared to HLA-G TCB monotherapy alone. No effect was seen for a combination of P1AF7977 treatment with anti-PD1 antibody (aPD1) and anti-LAG3 antibody (aLAG3) treatment, as compared to vehicle. Combination of 0.5 mg/kg HLA-G TCB with anti-PD1/anti-LAG3 bispecific antibody (PD1-LAG3), particularly at a anti-PD1/anti-LAG3 bispecific antibody concentration of 3 mg/kg, but also at the lower concentration of 1.5 mg/kg, showed increased Granzyme B (GZMB+) levels in CD3+ cells at treatment day 32, indicating sustained T cell activation, as compared to monotherapy groups and combination of HLA-G TCB treatment with anti-PD1 antibody and anti-LAG3 antibody treatment (FIG. 6B, bottom).

This study established the contribution of PD-1 and LAG3 inhibition by anti-PD1/anti-LAG3 bispecific antibody over a) single treatment with HLA-G-TCB, b) combination of HLA-G TCB and anti-PD-L1 antibody as well as c) combination of HLA-G TCB with anti-PD1 antibody and anti-LAG3 antibody in this model. In summary, the in vivo results reported here support the finding, that the combination of P1AF7977 with P1AA0927 shows synergistic activity resulting in enhanced T cell infiltration, activation and tumor growth inhibition.

1-22. (canceled)

23. A method for treating an HLA-G expressing cancer in a subject comprising administering to the subject an effective amount of an anti-HLA-G/anti-CD3 antibody and an effective amount of an anti-PD1/anti-LAG3 bispecific antibody, wherein the anti-PD1/anti-LAG3 bispecific antibody comprises a first antigen binding domain that specifically binds to programmed cell death protein 1 (PD1) and a second antigen binding domain that specifically binds to Lymphocyte activation gene-3 (LAG3), wherein the first antigen binding domain specifically binding to PD1 comprises a VH domain comprising
(i) CDR-H1 comprising the amino acid sequence of SEQ ID NO:1,
(ii) CDR-H2 comprising the amino acid sequence of SEQ ID NO:2, and
(iii) CDR-H3 comprising an amino acid sequence of SEQ ID NO:3; and
a VL domain comprising
(i) CDR-L1 comprising the amino acid sequence of SEQ ID NO:4;
(ii) CDR-L2 comprising the amino acid sequence of SEQ ID NO:5, and
(iii) CDR-L3 comprising the amino acid sequence of SEQ ID NO:6.

24. The method of claim 23, wherein the anti-PD1/anti-LAG3 bispecific antibody comprises a second antigen binding domain that specifically binds to LAG3 comprising
(a) a VH domain comprising
(i) CDR-H1 comprising the amino acid sequence of SEQ ID NO:11,
(ii) CDR-H2 comprising the amino acid sequence of SEQ ID NO:12, and
(iii) CDR-H3 comprising an amino acid sequence of SEQ ID NO:13; and
a VL domain comprising
(i) CDR-L1 comprising the amino acid sequence of SEQ ID NO:14,
(ii) CDR-L2 comprising the amino acid sequence of SEQ ID NO:15, and
(iii) CDR-L3 comprising the amino acid sequence of SEQ ID NO:16; or
(b) a VH domain comprising
(i) CDR-H1 comprising the amino acid sequence of SEQ ID NO:19,
(ii) CDR-H2 comprising the amino acid sequence of SEQ ID NO:20, and
(iii) CDR-H3 comprising an amino acid sequence of SEQ ID NO:21; and
a VL domain comprising
(i) CDR-L1 comprising the amino acid sequence of SEQ ID NO:22,
(ii) CDR-L2 comprising the amino acid sequence of SEQ ID NO:23, and
(iii) CDR-L3 comprising the amino acid sequence of SEQ ID NO:24.

25. The method of claim 23, wherein the anti-PD1/anti-LAG3 bispecific antibody comprises
a first Fab fragment specifically binding to PD1 comprising a VH domain comprising the amino acid sequence of SEQ ID NO:9 and a VL domain comprising the amino acid sequence of SEQ ID NO:10,
and a second Fab fragment specifically binding to LAG3 comprising a VH domain comprising the amino acid sequence of SEQ ID NO:17 and a VL domain comprising the amino acid sequence of SEQ ID NO:18.

26. The method of claim 23, wherein the anti-HLA-G/anti-CD3 bispecific antibody and the anti-PD1/anti-LAG3 bispecific antibody are administered together in a single composition or administered separately in two or more different compositions.

27. The method of claim 23, wherein the anti-HLA-G/anti-CD3 bispecific antibody and the anti-PD1/anti-LAG3 bispecific antibody are administered intravenously or subcutaneously.

28. The method of claim 23, wherein the anti-HLA-G/anti-CD3 bispecific antibody is administered concurrently with, prior to, or subsequently to the anti-PD1/anti-LAG3 bispecific antibody.

29. The method of claim 23, wherein the anti-PD1/anti-LAG3 bispecific antibody comprises an Fc domain that is an IgG Fc domain, and wherein the Fc domain comprises one or more amino acid substitutions that reduce binding to an Fc receptor.

30. The method of claim 23, wherein the anti-PD1/anti-LAG3 bispecific antibody comprises a first antigen-binding domain specifically binding to PD1 comprising the VH domain comprising the amino acid sequence of SEQ ID NO:9 and the VL domain comprising the amino acid sequence of SEQ ID NO:10.

31. The method of claim 23, wherein the anti-PD1/anti-LAG3 bispecific antibody comprises a second antigen-binding domain specifically binding to LAG3 comprising
(a) a VH domain comprising the amino acid sequence of SEQ ID NO:17 and a VL domain comprising the amino acid sequence of SEQ ID NO:18, or
(b) a VH domain comprising the amino acid sequence of SEQ ID NO:25 and a VL domain comprising the amino acid sequence of SEQ ID NO:26.

32. The method of claim 23, wherein the anti-PD1/anti-LAG3 bispecific antibody comprises a second antigen-binding domain specifically binding to LAG3 comprising
(a) a VH domain comprising the amino acid sequence of SEQ ID NO:27 and a VL domain comprising the amino acid sequence of SEQ ID NO:28, or
(b) a VH domain comprising the amino acid sequence of SEQ ID NO:29 and a VL domain comprising the amino acid sequence of SEQ ID NO:30, or
(c) a VH domain comprising the amino acid sequence of SEQ ID NO:31 and a VL domain comprising the amino acid sequence of SEQ ID NO:32, or
(d) a VH domain comprising the amino acid sequence of SEQ ID NO:33 and a VL domain comprising the amino acid sequence of SEQ ID NO:34, or
(e) a VH domain comprising the amino acid sequence of SEQ ID NO:64 and a VL domain comprising the amino acid sequence of SEQ ID NO:65.

33. The method of claim 23, wherein the anti-PD1/anti-LAG3 bispecific antibody comprises a first heavy chain comprising an amino acid sequence of SEQ ID NO:35, a first light chain comprising an amino acid sequence of SEQ ID NO:36, a second heavy chain comprising an amino acid sequence of SEQ ID NO:37, and a second light chain comprising an amino acid sequence of SEQ ID NO:38.

34. The method of claim 23, wherein the anti-HLA-G/anti-CD3 bispecific antibody comprises a first antigen binding domain specifically binding to CD3 comprising a heavy chain variable region (V_{H}CD3) comprising CDR-H1 sequence of SEQ ID NO:41, CDR-H2 sequence of SEQ ID NO:42, and CDR-H3 sequence of SEQ ID NO:43; and/or a light chain variable region (V_{L}CD3) comprising CDR-L1 sequence of SEQ ID NO:44, CDR-L2 sequence of SEQ ID NO:45, and CDR-L3 sequence of SEQ ID NO:46.

35. The method of claim 23, wherein the anti-HLA-G/anti-CD3 bispecific antibody comprises a second antigen binding domain specifically binding to HLA-G comprising a heavy chain variable region (V_{H}HLA-G) comprising CDR-H1 sequence of SEQ ID NO:49, CDR-H2 sequence of SEQ ID NO:50, and CDR-H3 sequence of SEQ ID NO:51; and/or a light chain variable region (V_{L}HLA-G) comprising CDR-L1 sequence of SEQ ID NO:52, CDR-L2 sequence of SEQ ID NO:53, and CDR-L3 sequence of SEQ ID NO:54.

36. A combination comprising an anti-PD1/anti-LAG3 bispecific antibody and an anti-HLA-G/anti-CD3 bispecific antibody, wherein the anti-PD1/anti-LAG3 bispecific antibody comprises a first antigen binding domain that specifically binds to programmed cell death protein 1 (PD1) and a second antigen binding domain that specifically binds to Lymphocyte activation gene-3 (LAG3), wherein the first antigen binding domain specifically binding to PD1 comprises a VH domain comprising
(i) CDR-H1 comprising the amino acid sequence of SEQ ID NO:1,
(ii) CDR-H2 comprising the amino acid sequence of SEQ ID NO:2, and
(iii) CDR-H3 comprising the amino acid sequence of SEQ ID NO:3; and
a VL domain comprising
(i) CDR-L1 comprising the amino acid sequence of SEQ ID NO:4;
(ii) CDR-L2 comprising the amino acid sequence of SEQ ID NO:5, and
(iii) CDR-L3 comprising the amino acid sequence of SEQ ID NO:6.
